(19) United States
(12) Patent Application Publication
(10) Pub. No.: US 2015/0038482 A1
(43) Pub. Date: Feb. 5, 2015

(54) ISOXAZOLE BETA-LACTAMASE INHIBITORS

(71) Applicant: CUBIST PHARMACEUTICALS, INC., LEXINGTON, MA (US)

(72) Inventors: Yu Gui Gu, Acton, MA (US); Yong He, Bedford, MA (US); Ning Yin, Lexington, MA (US); Dylan C. Alexander, Watertown, MA (US); Jason B. Cross, Acton, MA (US); Chester A. Metcalf, III, Needham, MA (US); Robert Busch, Wakefield, MA (US)

(21) Appl. No.: 14/505,438

(22) Filed: Oct. 2, 2014

Related U.S. Application Data
(63) Continuation of application No. 13/853,498, filed on Mar. 29, 2013.
(60) Provisional application No. 61/618,127, filed on Mar. 30, 2012.

(51) Int. Cl. C07D 471/08; A61K 31/439; A61K 31/426

(52) U.S. Cl. CPC C07D 471/08; A61K 31/426; A61K 31/439
USPC 514/210.15

(57) ABSTRACT

β-Lactamase inhibitor compounds (BLIs) are disclosed, including compounds that have activity against class A, class C or class D β-lactamases. Methods of manufacturing the BLIs, and uses of the compounds in the preparation of pharmaceutical compositions and antibacterial applications are also disclosed.

### RELATED APPLICATIONS

This application claims priority to U.S. Provisional Application No. 61/618,127, filed Mar. 30, 2012, and U.S. Provisional Application No. 61/790,248, filed Mar. 15, 2013. The entire contents of these applications are incorporated herein by reference in their entireties.

### TECHNICAL FIELD

This disclosure is directed to β-lactamase inhibitors (BLIs) which are effective as inhibitors of β-lactamases and, when used in combination with β-lactam antibiotics are useful in the treatment of bacterial infections. The compounds when combined with a β-lactam antibiotic are effective in treating infections caused by bacteria that are resistant to β-lactam antibiotics due to the presence of β-lactamases. Pharmaceutical compositions comprising such compounds, methods of using such compounds, and processes for preparing such compounds are also disclosed.

### BACKGROUND

Bacterial resistance to β-lactam antibiotics, especially in Gram-negative bacteria, is most commonly mediated by β-lactamases. β-lactamases are enzymes that catalyze the hydrolysis of the β-lactam ring, which inactivates the antibacterial activity of the β-lactam antibiotic and allows the bacteria to become resistant. Inhibition of the β-lactamase with a BLI slows or prevents degradation of the β-lactam antibiotic and restores β-lactam antibiotic susceptibility to β-lactamase producing bacteria. Many of these β-lactamases are not effectively inhibited by BLIs currently on the market rendering the β-lactam antibiotics ineffective in treating bacteria that produce these β-lactamases. There is an urgent need for novel BLIs that inhibit β-lactamases that are not effectively inhibited by the current clinical BLIs (e.g. KPC, class C and class D β-lactamases) and that could be used in combination with β-lactam antibiotics to treat infections caused by β-lactam resistant bacteria.

### SUMMARY OF INVENTION

The present invention provides, in one aspect, compounds of chemical formula (I), or pharmaceutically-acceptable salts thereof, which are BLIs and are useful in combination with β-lactam antibiotics for the treatment of bacterial infections.

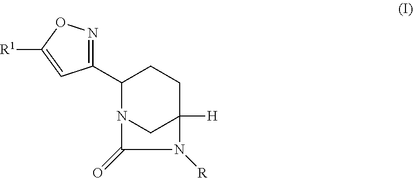

wherein
R is selected from

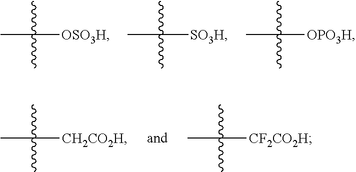

and
R¹ is selected from:
   a.

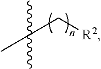

wherein R² is selected from

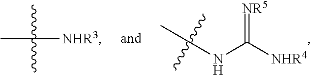

wherein each of R³, R⁴ and R⁵ is independently selected from hydrogen, (C₁-C₃)-alkyl, aminoalkyl, aminocycloalkyl, and hydroxyalkyl, and n is selected from 1, 2 and 3;
b.

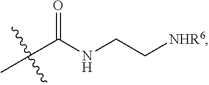

wherein R⁶ is selected from
H and

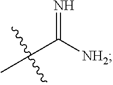 [SMILES: *C(=N)N]

C.

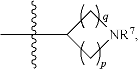

wherein R⁷ is selected from H, (C₁-C₃)-unsubstituted alkyl, amino-(C₂-C₃)-alkyl, amino cyclo alkyl, hydroxyalkyl, and

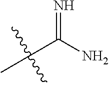 [SMILES: *C(=N)N]

and wherein each of p and q is independently selected from 1 and 2.

In another aspect, the invention provides compounds of chemical Formula (A-I) or a pharmaceutically acceptable salt thereof, which are BLIs and are useful in combination with β-lactam antibiotics for the treatment of bacterial infections.

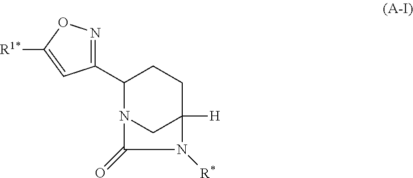

wherein
R* is selected from

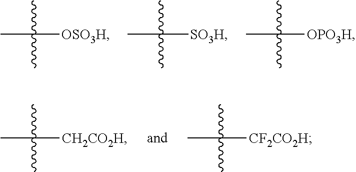

and
R¹* is selected from:
   a.

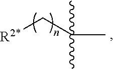

wherein R²* is selected from

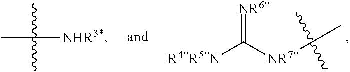

R³* is selected from hydrogen, (C₁-C₃)-alkyl, aminoalkyl,
aminocycloalkyl, hydroxyalkyl,

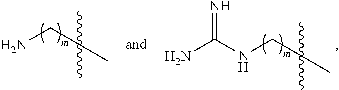

each of R⁴*, R⁵*, R⁶* and R⁷* is independently selected from hydrogen, (C₁-C₆)-alkyl, aminoalkyl, aminocycloalkyl, and hydroxyalkyl, provided that at least one of R⁴*, R⁵*, R⁶* and R⁷* is hydrogen,
n is selected from 1, 2, 3 and 4, and
m is selected from 1, 2 and 3;
b.

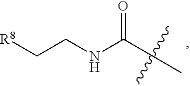

wherein R⁸ is selected from —NH(C₁-C₃)-alkyl and

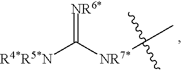

wherein each of R⁴*, R⁵*, R⁶* and R⁷* is as described previously;
c.

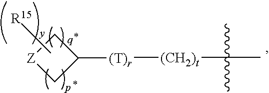

wherein Z is selected from CR⁹R¹⁰ and NR¹¹,
each of R⁹ and R¹⁰ is independently selected from H, NH₂, —NH(C₁-C₃)— alkyl and

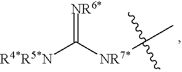

wherein each of R⁴*, R⁵*, R⁶* and R⁷* is as described previously,
alternatively, R⁹ and R¹⁰ together with the carbon to which they are attached, form a cycloalkyl or heterocyclyl ring containing 4-6 ring members,
R¹¹ is selected from H and

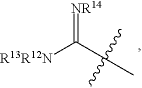

each of R R¹³ and R¹⁴ is independently selected from hydrogen, (C₁-C₆)-alkyl, aminoalkyl, aminocycloalkyl, and hydroxyalkyl, provided that at least one of R¹², R¹³ and R¹⁴ is hydrogen,
R¹⁵ is selected from NH₂ and

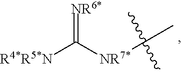

wherein each of R⁴*, R⁵*, R⁶* and R⁷* is as described previously,
each of p* and q* is independently selected from 0, 1, 2 and 3,
T is selected from NH and O,
t is selected from 0, 1, 2, 3, and 4, and
each of r and y is independently selected from 0 and 1;
d.

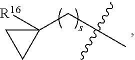

wherein R¹⁶ is selected from NH₂, —NH(C₁-C₃)-alkyl and

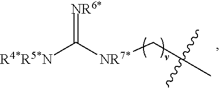

wherein each of R⁴*, R⁵*, R⁶* and R⁷* is as described previously,
s is selected from 0 and 1, and,
v is selected from 0, 1, 2, and 3;
e.

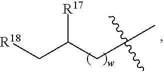

wherein R¹⁸ is selected from NH₂ and

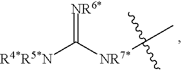

wherein each of R⁴*, R⁵*, R⁶* and R⁷* is as described previously,
R¹⁷ is selected from amino and hydroxyl, and
w is selected from 0 and 1;
f.

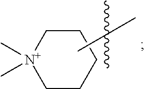

g.

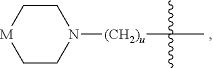

wherein M is selected from NR¹⁹, CR²⁰R²¹ and O,
wherein R¹⁹ is selected from H and

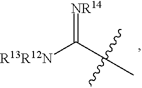

where each of R¹², R¹³ and R¹⁴ is as described previously,
each of R²⁰ and R²¹ is independently selected from H, NH₂ and

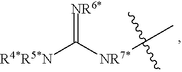

wherein each of R⁴*, R⁵*, R⁶* and R⁷* is as described previously, and
u is selected from 0, 1 and 2; and
h.

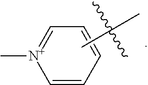

In one embodiment, the invention provides use of a compound of Formula I for inhibiting β-lactamases.

In one embodiment, the invention provides use of a compound of Formula A-I for inhibiting β-lactamases.

In one embodiment, the invention provides compounds of Formula I with high binding affinity for β-lactamase enzymes.

In one embodiment, the invention provides compounds of Formula A-I with high binding affinity for β-lactamase enzymes.

In one embodiment, the present invention also provides antibacterial compositions comprising compounds of Formula I and at least one β-lactam antibiotic.

In one embodiment, the present invention also provides antibacterial compositions comprising compounds of Formula A-I and at least one β-lactam antibiotic.

In one embodiment, the present invention provides pharmaceutical compositions comprising compounds of Formula I and at least one β-lactam antibiotic and methods of use thereof.

In one embodiment, the present invention provides pharmaceutical compositions comprising compounds of Formula A-I and at least one β-lactam antibiotic and methods of use thereof.

In one embodiment, the invention provides methods of use of the compounds of Formula I to treat bacterial infections in a subject.

In one embodiment, the invention provides methods of use of the compounds of Formula A-I to treat bacterial infections in a subject.

### BRIEF DESCRIPTION OF THE DRAWINGS

FIGS. 1A-1D show Table I, Representative Compounds of Formula A-II

FIGS. 2A-2B show Table II, Standard BLI potentiation MIC assay against a panel of isogenic and clinical strains expressing β-lactamases.

FIGS. 3A-3B show Table III, the synergy MIC of representative compounds of Formula II-A against a panel of isogenic and clinical strains expressing β-lactamases.

FIG. 4 shows Table IV, an assay to determine inhibition kinetics of representative compounds of Formula II-A for the KPC-2 β-lactamase.

FIGS. 5A-5B show Table V, Synergy MIC of Comparator Compounds Against a Panel of Isogenic and Clinical Strains Expressing β-lactamases

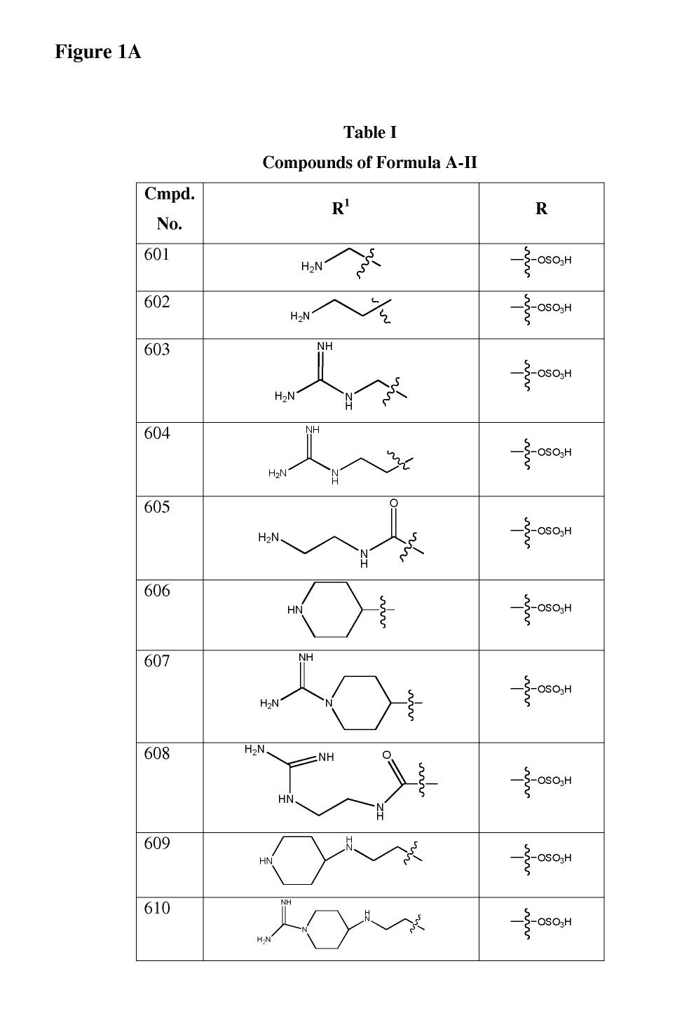

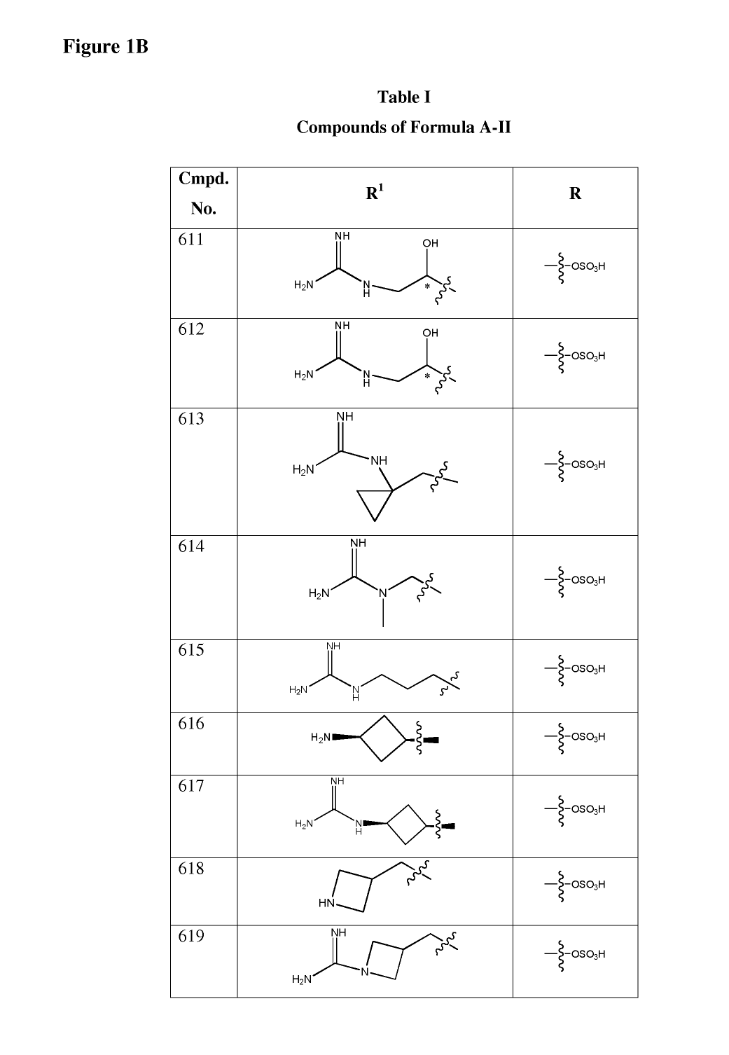

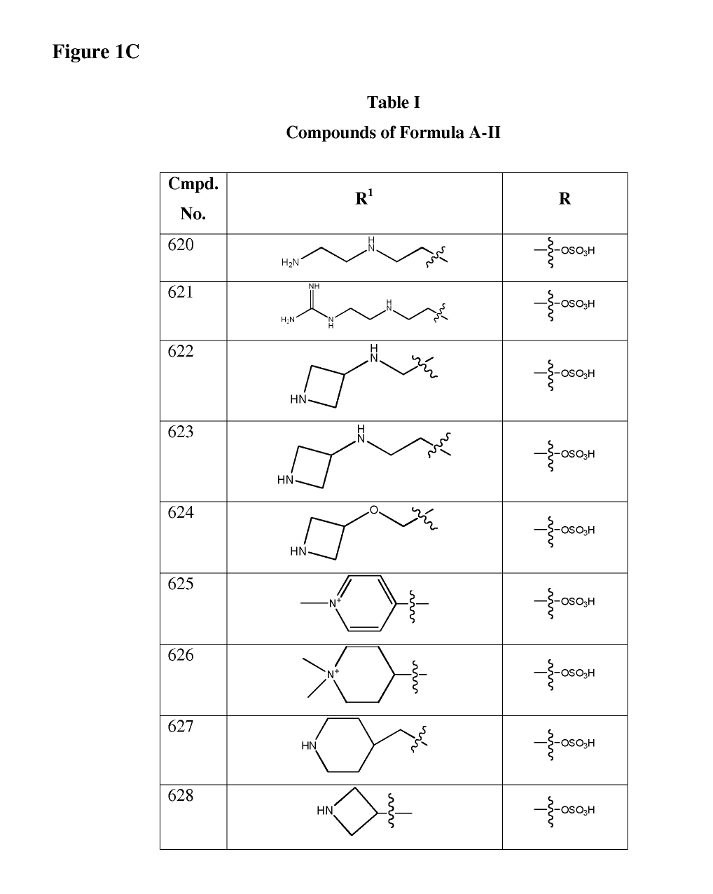

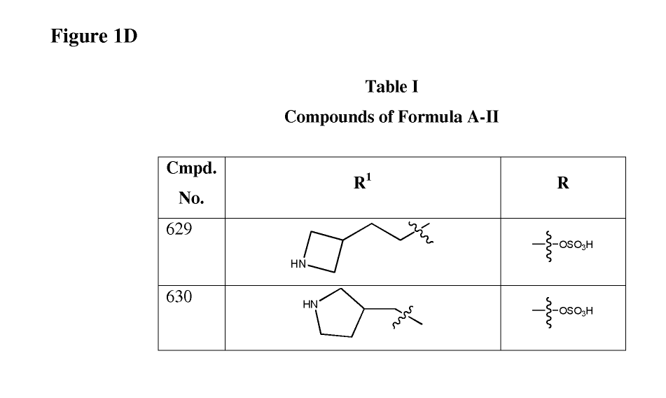

### DETAILED DESCRIPTION

### Definitions:

Molecular terms, when used in this application, have their common meaning unless otherwise specified.

The term “alkyl” is defined as a linear or branched, saturated radical having one to about twenty carbon atoms unless otherwise specified. Preferred alkyl radicals are “lower alkyl” radicals having one to about five carbon atoms. Examples of alkyl groups include, without limitation, methyl, ethyl, tert-butyl, isopropyl, and hexyl. A subset of the term alkyl is “(C₁-C₃)-unsubstituted alkyl” which is defined as an alkyl group that bears no substituent groups. Examples of (C₁-C₃)-unsubstituted alkyl groups include methyl, ethyl, propyl and isopropyl. It is understood that if a (C₁-C₃)-alkyl is “substituted” that one or more hydrogen atoms is replaced by a substitutent.

The term amino denotes a NH₂ radical.

The term “aminoalkyl” denotes an alkyl in which one or more of the alkyl hydrogen atoms has been replaced by an amino group.

The term “aminocycloalkyl” denotes a cycloalkyl in which one of the cycloalkyl hydrogen atoms has been replaced by an amino group.

The term “cycloalkyl” or “cycloalkyl ring” is defined as a saturated or partially unsaturated carbocyclic ring in a single or fused carbocyclic ring system having from three to twelve ring members. In a preferred embodiment, a cycloalkyl is a ring system having three to seven ring members. Examples of a cycloalkyl group include, without limitation, cyclopropyl, cyclobutyl, cyclohexyl, and cycloheptyl.

The term “hydroxyalkyl” denotes an alkyl radical in which one or more of the alkyl hydrogen atoms has been replaced by a hydroxyl group.

It will be understood by one of skill in the art that a [symbol] or — denote the point of attachment of a substituent group where indicated. For example

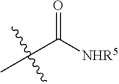

or —C(O)NHR⁵ represent that the point of attachment of the amide moiety is at the carbonyl carbon.

The functional classification of β-lactamases and terms “Class A”, “Class C”, and “Class D” β-lactamases are understood by one of skill in the art and are described in “Updated Functional Classification of β-Lactamases”, Bush, K.; Jacoby, G. A.; Antimicrob. Agents Chemother. 2010, 54, 969-976, herein incorporated by reference.

The salts of the compounds of the invention include acid addition salts and base addition salts. In a one embodiment, the salt is a pharmaceutically acceptable salt of the compound of Formula I. The term “pharmaceutically acceptable salts” embraces salts commonly used to form alkali metal salts and to form addition salts of free acids or free bases. The nature of the salt is not critical, provided that it is pharmaceutically-acceptable. Suitable pharmaceutically acceptable acid addition salts of the compounds of the invention may be prepared from an inorganic acid or an organic acid. Examples of such inorganic acids include, without limitation, hydrochloric, hydrobromic, hydroiodic, nitric, carbonic, sulfuric and phosphoric acid. Examples of appropriate organic acids may be selected from aliphatic, cycloaliphatic, aromatic, arylaliphatic, heterocyclic, carboxylic and sulfonic classes of organic acids, examples of which include, without limitation, formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, and galacturonic acid. Suitable pharmaceutically-acceptable base addition salts of compounds of the invention include, but are not limited to, metallic salts made from aluminum, calcium, lithium, magnesium, potassium, sodium and zinc or organic salts made from N,N′-dibenzylethylenediamine, chloroprocaine, choline, diethanolamine, ethylenediamine, N-methylglucamine, lysine and procaine. All of these salts may be prepared by conventional means from the corresponding compound of the invention by treating, for example, the compound of the invention with the appropriate acid or base.

The compounds of the invention can possess one or more asymmetric carbon atoms and are thus capable of existing in the form of optical isomers as well as in the form of racemic or non-racemic mixtures thereof. The compounds of the invention can be utilized in the present invention as a single isomer or as a mixture of stereochemical isomeric forms. Diastereoisomers, i.e., nonsuperimposable stereochemical isomers, can be separated by conventional means such as chromatography, distillation, crystallization or sublimation. The optical isomers can be obtained by resolution of the racemic mixtures according to conventional processes, for example by formation of diastereoisomeric salts by treatment with an optically active acid or base. Examples of appropriate acids include, without limitation, tartaric, diacetyltartaric, dibenzoyltartaric, ditoluoyltartaric and camphorsulfonic acid. The mixture of diastereomers can be separated by crystallization followed by liberation of the optically active bases from the optically active salts. An alternative process for separation of optical isomers includes the use of a chiral chromatography column optimally chosen to maximize the separation of the enantiomers. Still another available method involves synthesis of covalent diastereoisomeric molecules by treating compounds of the invention with an optically pure acid in an activated form or an optically pure isocyanate. The synthesized diastereoisomers can be separated by conventional means such as chromatography, distillation, crystallization or sublimation, and then hydrolyzed to obtain the enantiomerically pure compound. The optically active compounds of the invention can likewise be obtained by utilizing optically active starting materials. These isomers may be in the form of a free acid, a free base, an ester or a salt.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In one embodiment, isotopically-labeled compounds are useful in drug and/or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

The invention also embraces isolated compounds. An isolated compound refers to a compound which represents at least 10%, such as at least 20%, such as at least 50% and further such as at least 80% of the compound present in the mixture. In one embodiment, the compound, a pharmaceutically acceptable salt thereof, or a pharmaceutical composition comprising the compound exhibits detectable (i.e. statistically significant) activity when tested in conventional biological assays such as those described herein.

### β-Lactamase Inhibitors (BLIs)

In one aspect, the invention provides compounds of Formula I or pharmaceutically-acceptable salts thereof:

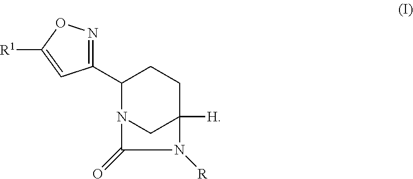

Substituent R of Formula I is selected from

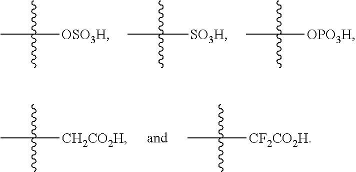

In a preferred embodiment, R is

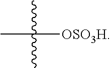

The group R¹ of Formula I is selected from:
   a.

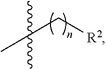

wherein R² is selected from

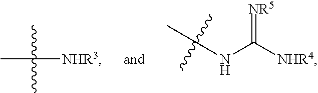

wherein each of R³, R⁴ and R⁵ is independently selected from hydrogen, (C₁-C₃)-alkyl, aminoalkyl, aminocycloalkyl, and hydroxyalkyl, and n is selected from 1, 2 and 3;
b.

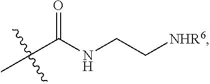

wherein R⁶ is selected from
H and

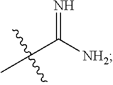 [SMILES: *C(=N)N]

c.

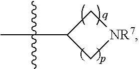

wherein R⁷ is selected from H, (C₁-C₃)-unsubstituted alkyl, amino-(C₂-C₃)-alkyl, amino cyclo alkyl, hydroxyalkyl, and

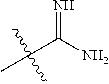 [SMILES: *C(=N)N]

and wherein each of p and q is independently selected from 1 and 2.

In one aspect of the invention n is 1. In another aspect of the invention n is 2. In another aspect of the invention n is 3.

In one aspect R¹ is selected from —CH₂NH₂—, —CH₂CH₂NH₂, —CONH(CH₂)₂NH₂,

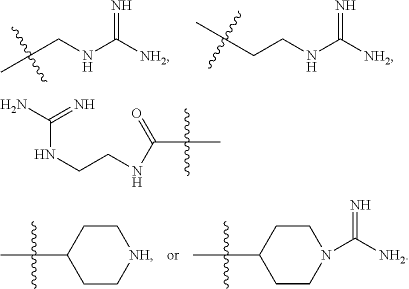 [SMILES: *CNC(=N)N ; *CCNC(=N)N ; *C(=O)NCCNC(=N)N ; *C1CCNCC1 ; "or" ; *C1CCN(C(=N)N)CC1]

In one embodiment of the invention, R¹ is selected from

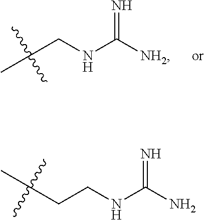 [SMILES: *CNC(=N)N ; "or" ; *CCNC(=N)N]

In one embodiment of the invention, the compounds of the invention are of the stereochemistry disclosed in Formula II.

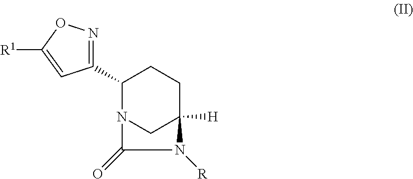

In another embodiment of the invention, the compound is of Formula II and R is —OSO₃H and R¹ is —CH₂NH₂.

In another embodiment of the invention, the compound is of Formula II and R is —OSO₃H and R¹ is —CH₂CH₂NH₂.

In another embodiment of the invention, the compound is of Formula II and R is —OSO₃H and R¹ is —CONH(CH₂)₂NH₂.

In another embodiment of the invention, the compound is of Formula II and R is —OSO₃H and R¹ is

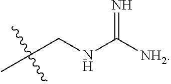 [SMILES: *CNC(=N)N]

In another embodiment of the invention, the compound is of Formula II and R is —OSO₃H and R¹ is

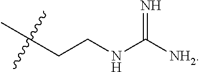 [SMILES: *CCNC(=N)N]

In another embodiment of the invention, the compound is of Formula II and R is —OSO₃H and R¹ is

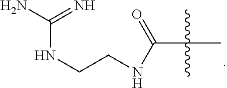 [SMILES: *C(=O)NCCNC(=N)N]

In another embodiment of the invention, the compound is of Formula II and R is —OSO₃H and R¹ is

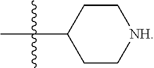 [SMILES: *C1CCNCC1]

In another embodiment of the invention, the compound is of Formula II and R is —OSO₃H and R¹ is

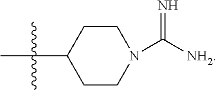 [SMILES: *C1CCN(C(=N)N)CC1]

Preferred compounds of Formula I are the compounds:

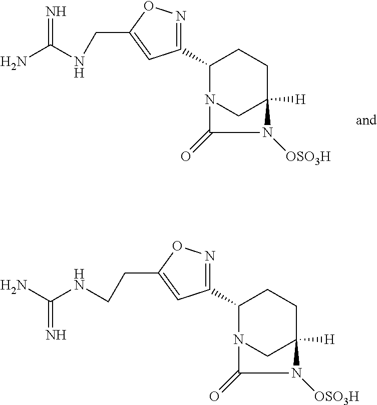

It will be understood by one of skill in the art that depending on the nature of R¹ and R, compounds of Formula I may exist in a salt or zwitterionic form.

In one aspect, the invention provides compounds of Formula A-I or pharmaceutically-acceptable salts thereof:

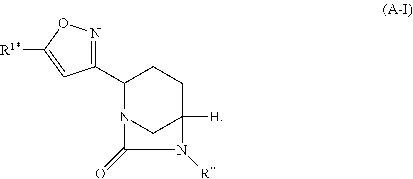

Substituent R* of Formula A-I is selected from

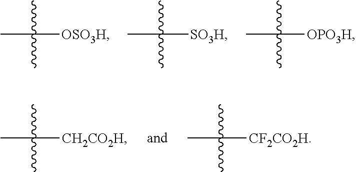

In a preferred embodiment, R* is

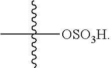

The group R¹* is selected from:
a.

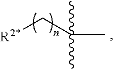

wherein R²* is selected from

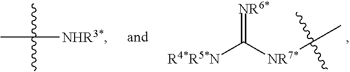

R³* is selected from hydrogen, (C₁-C₃)-alkyl, aminoalkyl, aminocycloalkyl, hydroxyalkyl,

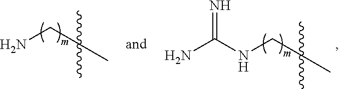

each of R⁴*, R⁵*, R⁶* and R⁷* is independently selected from hydrogen, (C₁-C₆)-alkyl, aminoalkyl, aminocycloalkyl, and hydroxyalkyl, provided that at least one of R⁴*, R⁵*, R⁶* and R⁷* is hydrogen,
n is selected from 1, 2, 3 and 4, and
m is selected from 1, 2 and 3;
b.

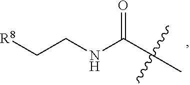

wherein R⁸ is selected from —NH(C₁-C₃)-alkyl and

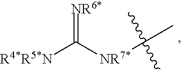

wherein each of R⁴*, R⁵*, R⁶* and R⁷* is as described previously;
c.

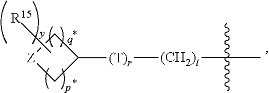

wherein Z is selected from CR⁹R¹⁰ and NR¹¹,
each of R⁹ and R¹⁰ is independently selected from H, NH₂, —NH(C₁-C₃)-alkyl and

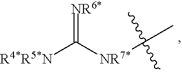

wherein each of R⁴*, R⁵*, R⁶* and R⁷* is as described previously,
alternatively, R⁹ and R¹⁰ together with the carbon to which they are attached, form a cycloalkyl or heterocyclyl ring containing 4-6 ring members, R¹¹ is selected from H and

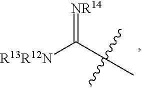

each of R¹², R¹³ and R¹⁴ is independently selected from hydrogen, (C₁-C₆)-alkyl, aminoalkyl, aminocycloalkyl, and hydroxyalkyl, provided that at least one of R¹², R¹³ and R¹⁴ is hydrogen,
R¹⁵ is selected from NH₂ and

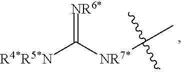

wherein each of R⁴*, R⁵*, R⁶* and R⁷* is as described previously,
each of p* and q* is independently selected from 0, 1, 2 and 3,
T is selected from NH and O,
t is selected from 0, 1, 2, 3, and 4, and
each of r and y is independently selected from 0 and 1;
d.

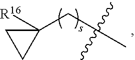

wherein R¹⁶ is selected from NH₂, —NH(C₁-C₃)-alkyl and

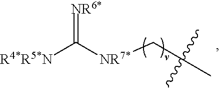

wherein each of R⁴*, R⁵*, R⁶* and R⁷* is as described previously,
s is selected from 0 and 1, and,
v is selected from 0, 1, 2, and 3;
e.

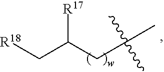

wherein R¹⁸ is selected from NH₂ and

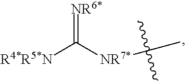

wherein each of R⁴*, R⁵*, R⁶* and R⁷* is as described previously,
R¹⁷ is selected from amino and hydroxyl, and
w is selected from 0 and 1;
f.

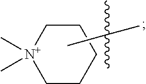

g.

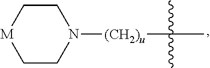

wherein M is selected from NR¹⁹, CR²⁰R²¹ and O,
wherein R¹⁹ is selected from H and

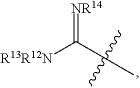

where each of R¹², R¹³ and R¹⁴ is as described previously,
each of R²⁰ and R²¹ is independently selected from H, NH₂ and

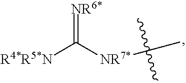

wherein each of R⁴*, R⁵*, R⁶* and R⁷* is as described previously, and
u is selected from 0, 1 and 2; and
h.

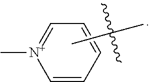

In one aspect of the invention R¹* is selected from

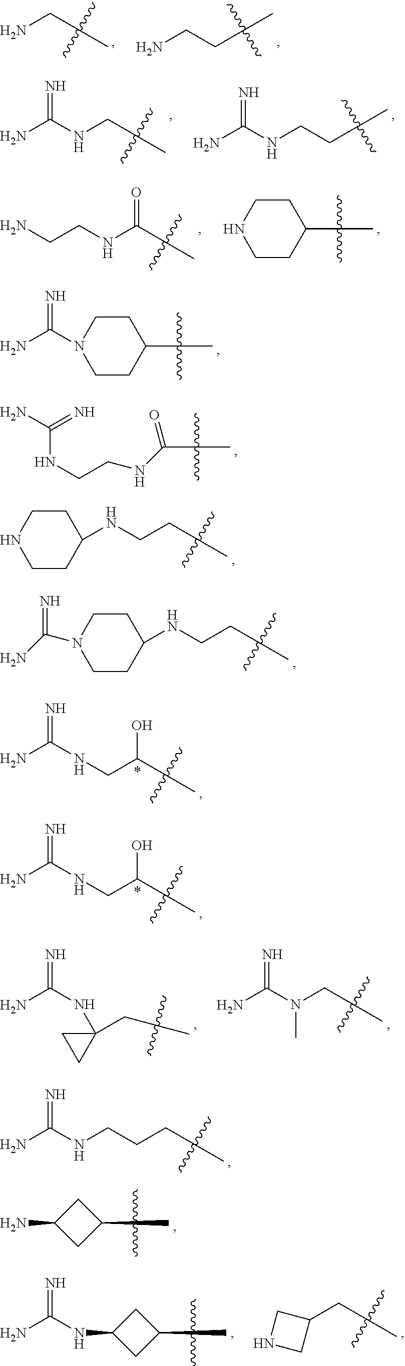

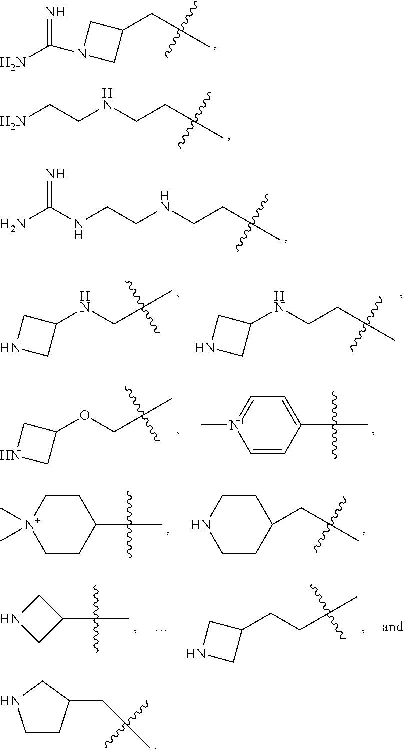

In one embodiment of the invention R¹* is selected from

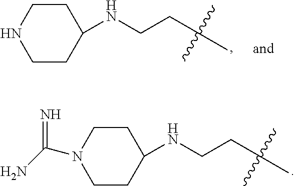 [SMILES: *CCNC1CCNCC1 ; *CCNC1CCN(C(=N)N)CC1]

In one embodiment of the invention, the compounds of the invention are of the stereochemistry disclosed in Formula A-II.

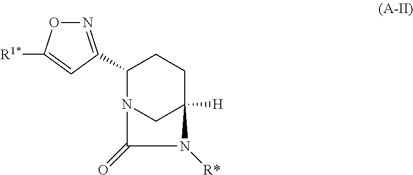

In another embodiment of the invention, R* and R¹* are chosen from the substituents listed in Table I (See FIG. I). Preferred compounds of Formula A-I are

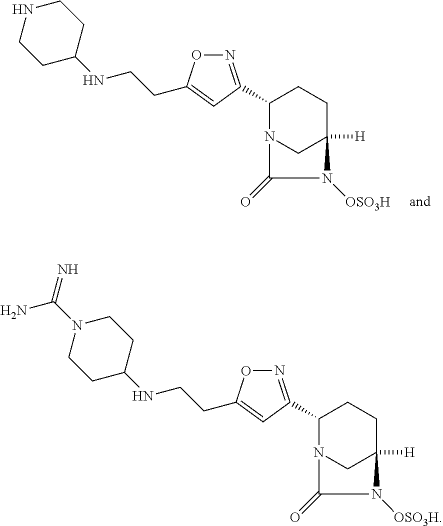

It will be understood by one of skill in the art that depending on the nature of R¹* and R*, compounds of Formula I may exist in a salt or zwitterionic form.

### Enzyme Inhibition and Binding Affinity

The compounds of the invention (e.g. compounds of Formula I, compounds of Formula A-I, compounds of Formula II, compounds of Formula A-II) are effective in inhibiting β-lactamase. In one aspect of the invention the compounds of Table I are effective β-lactamase inhibitors. In one aspect the compound

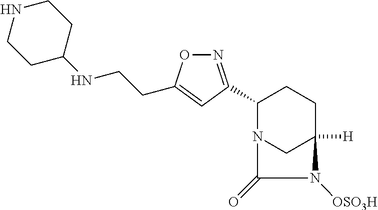

is effective in inhibiting β-lactamase. In one aspect the compound

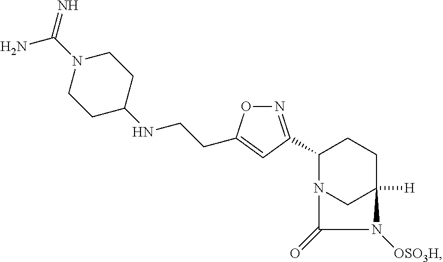

is effective in inhibiting β-lactamase

When used in combination with β-lactam antibiotics, the compounds of the invention (e.g. compounds of Formula I, compounds of Formula A-I, compounds of Formula II, compounds of Formula A-II) potentiate the activity of the β-lactam antibiotic against microorganisms that are normally resistant to β-lactam antibiotics due to the presence of a β-lactamase or multiple β-lactamases.

In one aspect of the invention the compounds of the invention (e.g. compounds of Formula I, compounds of Formula A-I, compounds of Formula II, compounds of Formula A-II) inhibit β-lactamases selected from class A, class C or class D β-lactamases. In one aspect of the invention the compounds of Formula I, inhibit β-lactamases selected from class A, class C or class D β-lactamases. In one aspect of the invention the compounds of Formula A-I inhibit β-lactamases selected from class A, class C or class D β-lactamases. In one aspect of the invention the compounds of Formula II inhibit β-lactamases selected from class A, class C or class D β-lactamases. In one aspect of the invention the compounds of Formula A-II inhibit β-lactamases selected from class A, class C or class D β-lactamases. In one aspect of the invention the compounds of Table I inhibit β-lactamases selected from class A, class C or class D β-lactamases. In one aspect of the invention the compound of Formula

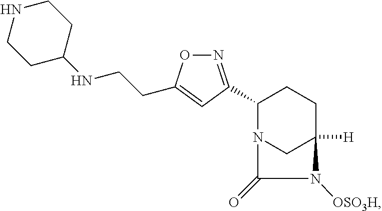

inhibits β-lactamases selected from class A, class C or class D β-lactamases. In one aspect of the invention the compound of Formula

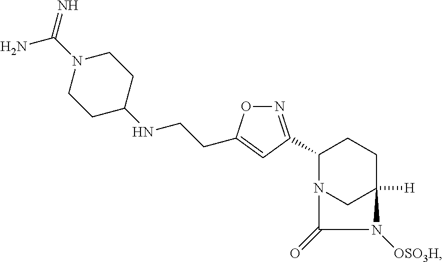

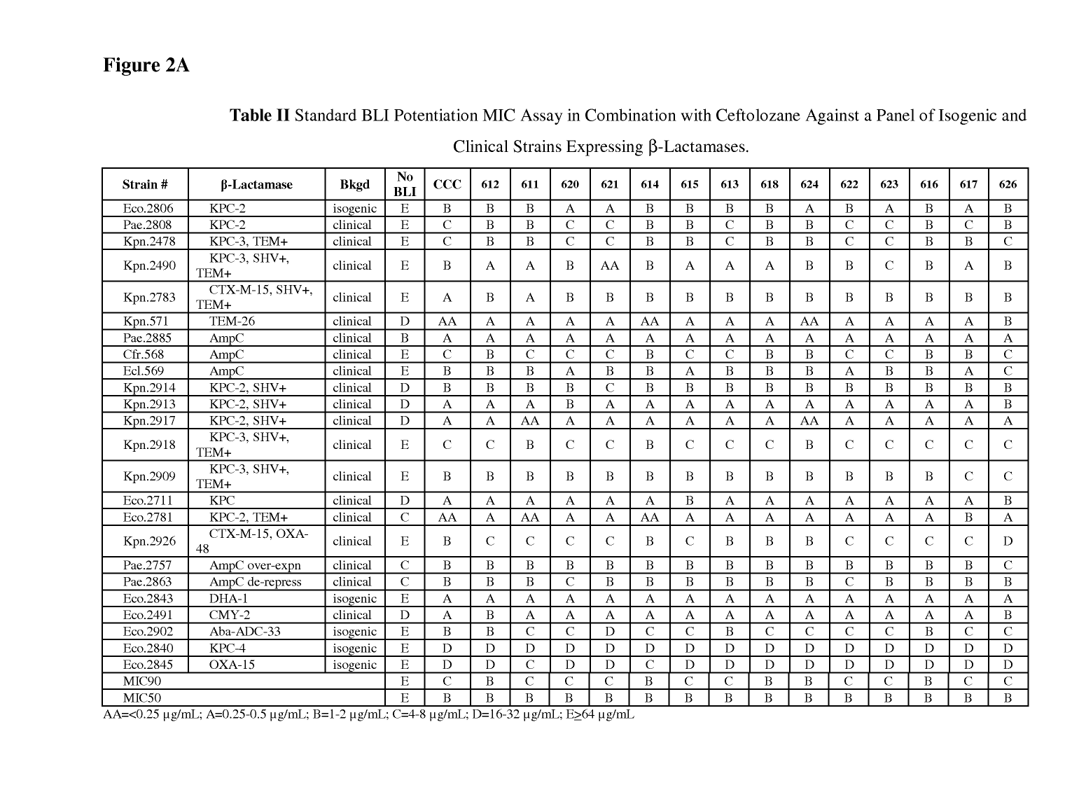

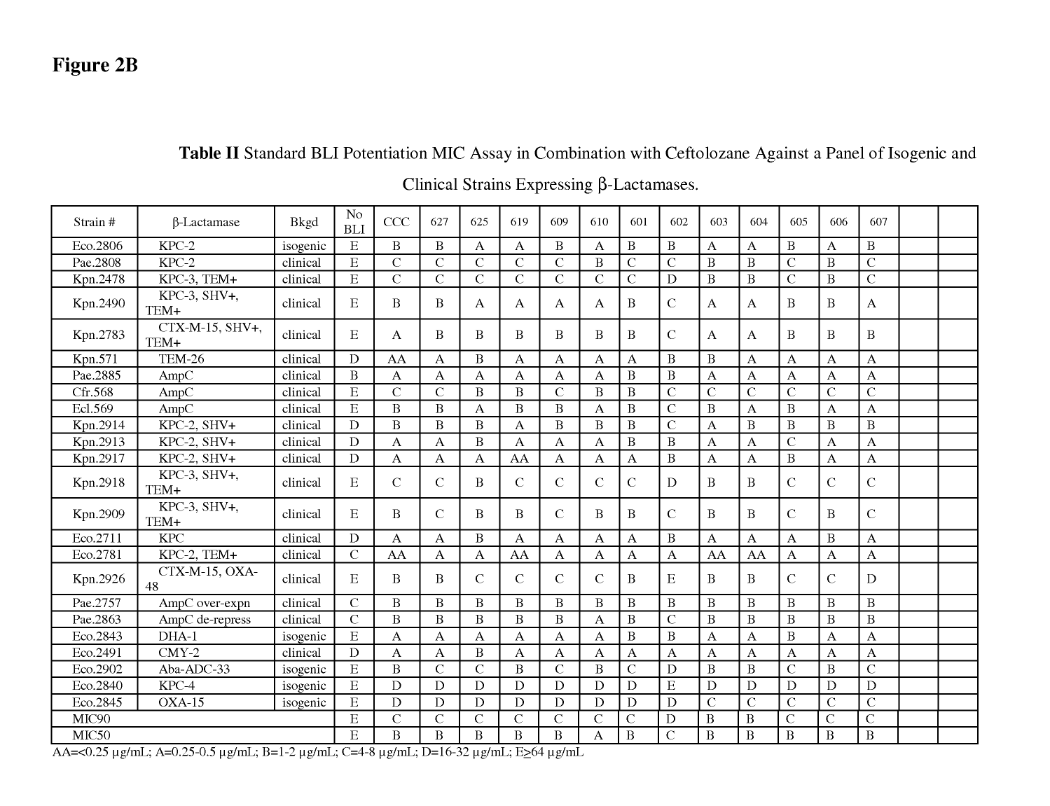

inhibits β-lactamases selected from class A, class C or class D β-lactamases. Class A β-lactamases for example, include, but are not limited to, TEM, SHV, CTX-M, KPC, GES, VEB, SME, and GEX. In a preferred aspect of the invention, the compounds of the invention (e.g. compounds of Formula I, compounds of Formula A-I, compounds of Formula II, compounds of Formula A-II) inhibit KPC β-lactamases. In a preferred aspect of the invention, the compounds of Formula I inhibit KPC β-lactamases. In a preferred aspect of the invention, the compounds of Formula A-I inhibit KPC β-lactamases. In a preferred aspect of the invention, the compounds of Formula II inhibit KPC β-lactamases. In a preferred aspect of the invention, the compounds of Formula A-II inhibit KPC β-lactamases. More preferably the compounds of the invention (e.g. compounds of Formula I, compounds of Formula A-I, compounds of Formula II, compounds of Formula A-II) inhibit KPC-2 or KPC-3 β-lactamases. More preferably the compounds of Formula I inhibit KPC-2 or KPC-3 β-lactamases. More preferably the compounds of Formula A-I inhibit KPC-2 or KPC-3 β-lactamases. More preferably the compounds of Formula II inhibit KPC-2 or KPC-3 β-lactamases. More preferably the compounds of Formula A-II inhibit KPC-2 or KPC-3 β-lactamases. In one aspect of the invention, the compounds of the invention (e.g. compounds of Formula I, compounds of Formula A-I, compounds of Formula II, compounds of Formula A-II) inhibit KPC-2 or KPC-3 β-lactamases in clinical strains (FIG. 2, Table II). In one aspect of the invention, the compounds of Formula I inhibit KPC-2 or KPC-3 β-lactamases in clinical strains (FIG. 2, Table II). In one aspect of the invention, the compounds of Formula A-I inhibit KPC-2 or KPC-3 β-lactamases in clinical strains (FIG. 2, Table II). In one aspect of the invention, the compounds of Formula II inhibit KPC-2 or KPC-3 β-lactamases in clinical strains (FIG. 2, Table II). In one aspect of the invention, the compounds of Formula A-II inhibit KPC-2 or KPC-3 β-lactamases in clinical strains (FIG. 2, Table II). Class C β-lactamases for example, include, but are not limited to chromosomal AmpCs, and plasmid based ACC, DHA, CMY, FOX, ACT, MIR, LAT, MOX β-lactamases. Class D β-lactamase enzymes, for example, include, but are not limited to oxacillinases or OXA β-lactamases. In a preferred aspect of the invention, the compounds of the invention (e.g. compounds of Formula I, compounds of Formula A-I, compounds of Formula II, compounds of Formula A-II) inhibit OXA-15 β-lactamases. In a preferred aspect of the invention, the compounds of Formula I inhibit OXA-15 β-lactamases. In a preferred aspect of the invention, the compounds of Formula A-I inhibit OXA-15 β-lactamases. In a preferred aspect of the invention, the compounds of Formula II inhibit OXA-15 β-lactamases. In a preferred aspect of the invention, the compounds of Formula A-II inhibit OXA-15 β-lactamases.

Unless otherwise indicated, the activity of the BLI compounds can be described by the MIC value obtained from a Synergy MIC assay or a BLI potentiation assay (e.g. as described herein), both of which are run in the presence of a β-lactam. The lower the sMIC or MIC value the more active the BLI, regardless of the mechanism of action of the BLI compound (e.g., including inhibition of β-lactamases by the BLI or any other mechanism of action or combination of mechanisms of action). The sMIC and BLI potentiation assay data supports that the compounds of the invention (e.g. compounds of Formula I, compounds of Formula A-I, compounds of Formula II, compounds of Formula A-II) potentiate (i.e. make more potent) the activity of the β-lactam antibiotic against β-lactamase producing strains by inhibiting the β-lactamase.

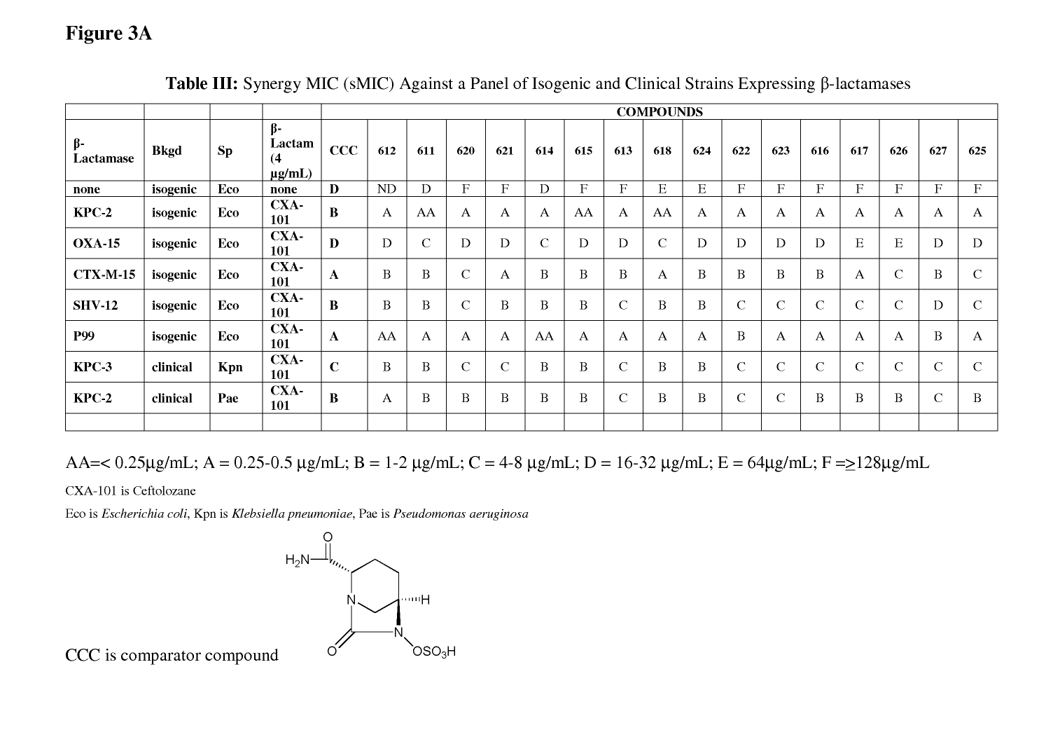

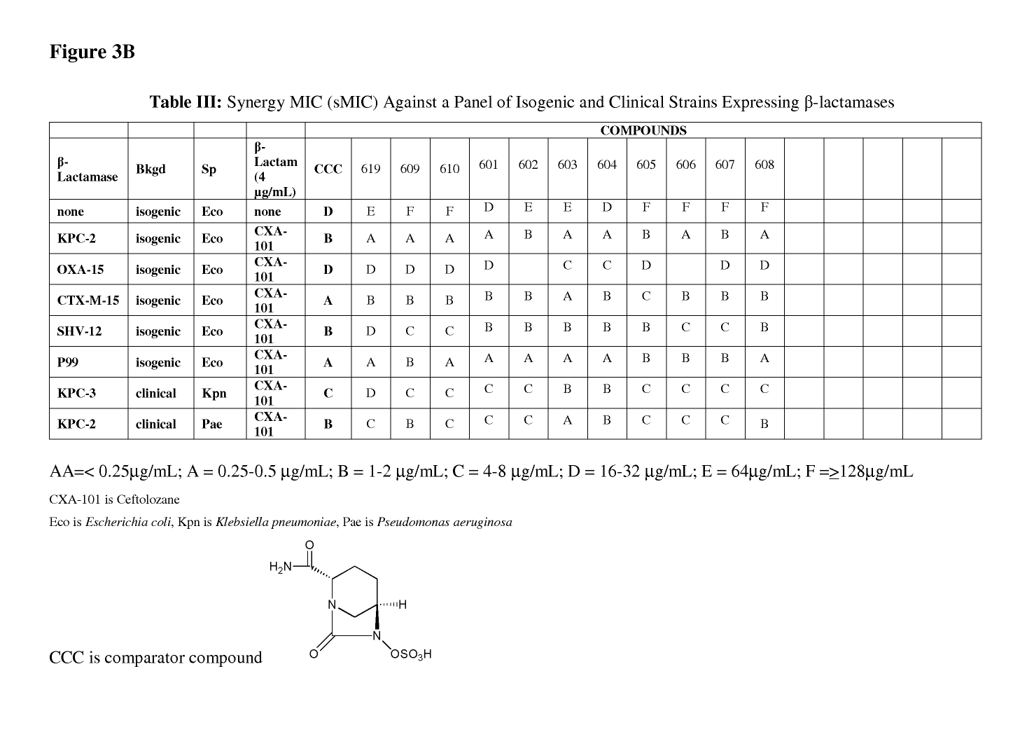

In one embodiment, the BLI activity is measured by growth inhibition of a β-lactamase producing bacterial strains in a Synergy MIC (sMIC) assay. Preferably, the sMIC value for the compounds of the invention (e.g. compounds of Formula I, compounds of Formula A-I, compounds of Formula II, compounds of Formula A-II) is 8 μg/mL or less. In a more preferred aspect of the invention, the sMIC value for the compounds of the invention (e.g. compounds of Formula I, compounds of Formula A-I, compounds of Formula II, compounds of Formula A-II) is 4 μg/mL to 8 μg/mL. In an even more preferred aspect of the invention, the sMIC value for the compounds of the invention (e.g. compounds of Formula I, compounds of Formula A-I, compounds of Formula II, compounds of Formula A-II) is 1 to 2 μg/mL. In a still more preferred aspect of the invention, the sMIC value for the compounds of the invention (e.g. compounds of Formula I, compounds of Formula A-I, compounds of Formula II, compounds of Formula A-II) is 0.2 to 0.5 μg/mL. Synergy MICs for representative compounds of the invention are described in Table III (See FIG. 3). It will be understood by one of skill in the art that the growth inhibition of β-lactamase producing strains can also be measured by a checkerboard synergy assay like that disclosed in International Patent Application Number WO 2008/039420 or a standard BLI potentiation assay using a fixed concentration of BLI.

In one embodiment, the BLI activity is measured by growth inhibition of a β-lactamase producing bacterial strains in a standard BLI potentiation assay using a fixed concentration of BLI. Preferably, the MIC value for the compounds of the invention (e.g. compounds of Formula I, compounds of Formula A-I, compounds of Formula II, compounds of Formula A-II) is 8 μg/mL or less. In a more preferred aspect of the invention, the MIC value for the compounds of the invention (e.g. compounds of Formula I, compounds of Formula A-I, compounds of Formula II, compounds of Formula A-II) is 4 to 8 μg/mL. In an even more preferred aspect of the invention, the MIC value for the compounds of the invention (e.g. compounds of Formula I, compounds of Formula A-I, compounds of Formula II, compounds of Formula A-II) is 1 to 2 μg/mL. In a still more preferred aspect of the invention, the MIC value for the compounds of the invention (e.g. compounds of Formula I, compounds of Formula A-I, compounds of Formula II, compounds of Formula A-II) is 0.2 μg/mL to 0.5 μg/mL.

The compounds of the present invention (e.g. compounds of Formula I, compounds of Formula A-I, compounds of Formula II, compounds of Formula A-II) have a broad spectrum of activity across a wide variety of β-lactamase producing bacteria. It was surprisingly found that the compounds of the present invention (e.g. compounds of Formula I, compounds of Formula A-I, compounds of Formula II, compounds of Formula A-II) are active in potentiating activity of β-lactam antibiotics, in particular, Ceftolozane, against strains expressing class D β-lactamase OXA-15 β-lactamase. Currently marketed BLIs inhibit most of the class A β-lactamases, but poorly inhibit class A KPCβ-lactamases and class C β-lactamases and have variable success in inhibiting penicillinase and carbapenemase-type class D β-lactamases. The compounds of the present invention (e.g. compounds of Formula I, compounds of Formula A-I, compounds of Formula II, compounds of Formula A-II) are active against a wide variety of bacterial strains that express class A and C β-lactamases and also, surprisingly are active against bacterial strains that express the class D cephalosporinase OXA-15 (Tables II and III). This increased activity against the class D β-lactamase is critical because differential effectiveness against different types of β-lactamase producing bacteria is necessary in order to effectively use β-lactam antibiotics to treat resistant strains of bacteria (vide infra).

In one embodiment, the compounds the invention (e.g. compounds of Formula I, compounds of Formula A-I, compounds of Formula II, compounds of Formula A-II) are unexpectedly more active against bacterial strains that express OXA-15 β-lactamases than the most structurally similar compound, Avibactam (comparator compound CCC). Compounds that are more active than Avibactam against bacterial strains that express the class D cephalosporinase OXA-15 are, for example, compounds 603, 604, 611, 614, 618.

In one embodiment, the compounds of the invention (e.g. compounds of Formula I, compounds of Formula A-I, compounds of Formula II, compounds of Formula A-II) are unexpectedly more active against and/or show broader spectrum of activity against bacterial strains that express KPC β-lactamases than the most structurally similar compound, Avibactam. Compounds that are more active than Avibactam for at least one, bacterial strain that expresses KPC β-lactamase and/or show a better spectrum of activity against bacterial strains that express KPC β-lactamases than Avibactam are, for example, compounds 601, 602, 603, 604, 605, 606, 607, 608, 609, 610, 611, 612, 613, 614, 615, 616, 617, 618, 620, 621, 622, 623, 624, 625, 626, and 627.

In another aspect of the invention, the compounds of the invention (e.g. compounds of Formula I, compounds of Formula A-I, compounds of Formula II, compounds of Formula A-II) have high binding affinity for the β-lactamase enzyme. Consequently these compounds are better inhibitors of the β-lactamase enzyme. The inhibition kinetics of the compounds of the invention (e.g. compounds of Formula I, compounds of Formula A-I, compounds of Formula II, compounds of Formula A-II) was measured according to the procedure outlined in Example 37. The compounds of the invention (e.g. compounds of Formula I, compounds of Formula A-I, compounds of Formula II, compounds of Formula A-II) have a high binding affinity for the β-lactamase enzyme.

In one embodiment the compounds of the invention (e.g. compounds of Formula I, compounds of Formula A-I, compounds of Formula II, compounds of Formula A-II) have a binding affinity of 1000-5000 mM⁻¹s⁻¹. Compounds that have a binding affinity of 1000-5000 mM⁻¹s⁻¹ are, for example, compound 604 and 608 (Table IV).

In one embodiment the compounds of the invention (e.g. compounds of Formula I, compounds of Formula A-I, compounds of Formula II, compounds of Formula A-II) have a binding affinity of 100-999 mM⁻¹s⁻¹. Compounds that have a binding affinity of 100-999 mM⁻¹s⁻¹ are, for example, compounds 601, 603, 605, 606, 607, 609, 610, 611, 612, 613, 614, 615, 616, 617, 618, 619, 620, 621, 622, 623, 624, 625, 626, and 627 (Table IV).

In one embodiment the compounds of the invention (e.g. compounds of Formula I, compounds of Formula A-I, compounds of Formula II, compounds of Formula A-II) have a binding affinity of 1-99 mM⁻¹s⁻¹. Compounds that have a binding affinity of 1-99 mM⁻¹s⁻¹ are, for example, 602 (Table IV).

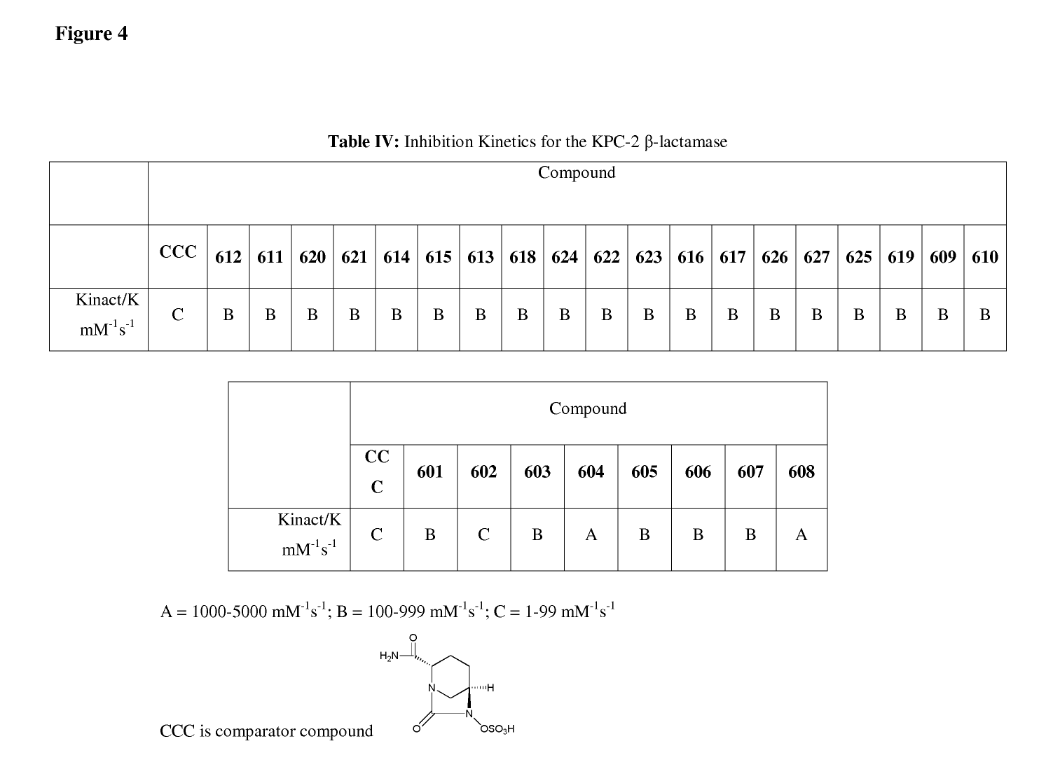

It was surprisingly found that the compounds of the present invention have a higher binding affinity for the β-lactamase enzyme than the closest structural comparator Avibactam (Table IV, See FIG. 4).

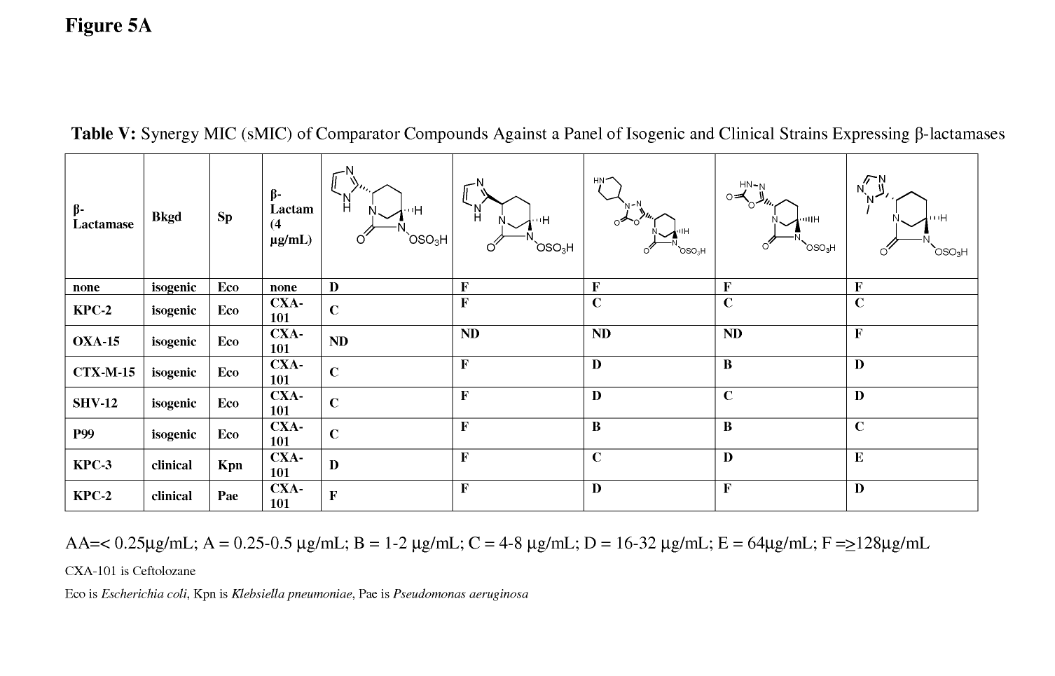

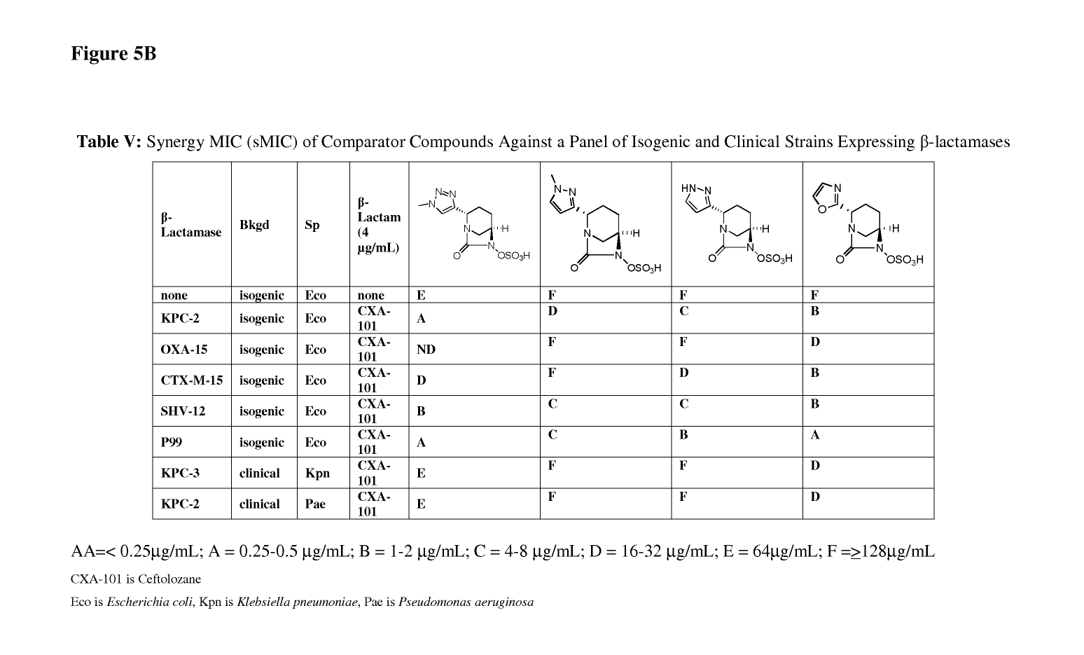

The compounds of the invention were also shown to be better BLIs than other comparator compounds as shown in FIG. 5.

### Pharmaceutical Compositions Comprising the Compounds of the Invention and Use Thereof

Another object of the invention is pharmaceutical compositions or formulations comprising compounds the invention (e.g. compounds of Formula I, compounds of Formula A-I, compounds of Formula II, compounds of Formula A-II), or salts thereof, preferably further comprising a β-lactam antibiotic. In one embodiment of the invention is pharmaceutical compositions or formulations comprising compounds of Formula I, or salts thereof, preferably further comprising a β-lactam antibiotic. In one embodiment of the invention is pharmaceutical compositions or formulations comprising compounds of Formula A-I, or salts thereof, preferably further comprising a β-lactam antibiotic. In one embodiment of the invention is pharmaceutical compositions or formulations comprising compounds of Formula II, or salts thereof, preferably further comprising a β-lactam antibiotic. In one embodiment of the invention is pharmaceutical compositions or formulations comprising compounds of Formula A-II, or salts thereof, preferably further comprising a β-lactam antibiotic. In one embodiment of the invention is pharmaceutical compositions or formulations comprising compounds of Table I. In one embodiment of the invention is pharmaceutical compositions or formulations comprising compounds of Formula

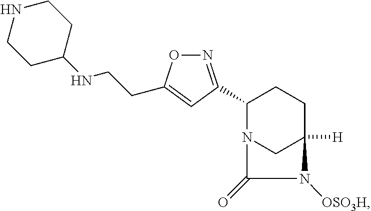

or salts thereof, preferably further comprising a β-lactam antibiotic. In one embodiment of the invention is pharmaceutical compositions or formulations comprising compounds of Formula

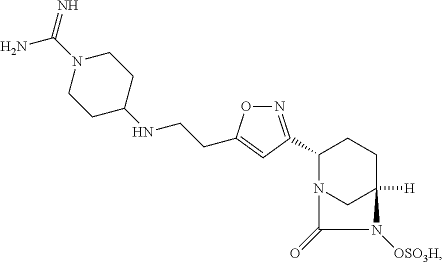

or salts thereof, preferably further comprising a β-lactam antibiotic.

The pharmaceutical compositions can be formulated for oral, intravenous, intramuscular, subcutaneous or parenteral administration for the therapeutic or prophylactic treatment of diseases, such as bacterial infections. Preferably, the pharmaceutical composition is formulated for intravenous administration.

The pharmaceutical preparations disclosed herein may be prepared in accordance with standard procedures and are administered at dosages that are selected to reduce, prevent or eliminate infection (see, e.g., Remington's Pharmaceutical Sciences, Mack Publishing Company, Easton, Pa. and Goodman and Gilman's “The Pharmaceutical Basis of Therapeutics,” Pergamon Press, New York, N.Y., the contents of which are incorporated herein by reference, for a general description of the methods for administering various antimicrobial agents for human therapy).

The pharmaceutical compositions can comprise one or more of the compounds disclosed herein (e.g. one or more compounds of Formula I, compounds of Formula A-I, compounds of Formula II, compounds of Formula A-II), preferably a compound of Formula A-I or Formula A-II, in conjunction with a β-lactam antibiotic, in association with one or more nontoxic, pharmaceutically-acceptable carriers and/or diluents and/or adjuvants and/or excipients. As used herein, the phrase “pharmaceutically-acceptable carrier” refers to any and all solvents, dispersion media, coatings, antibacterial and antifungal agents, isotonic and absorption delaying agents, and the like, that are compatible with pharmaceutical administration. The use of such media and agents for pharmaceutically active substances is well known in the art. Non-limiting examples of carriers and excipients include corn starch or gelatin, lactose, sucrose, microcrystalline cellulose, kaolin, mannitol, dicalcium phosphate, sodium chloride and alginic acid. The compositions may contain croscarmellose sodium, microcrystalline cellulose, corn starch, sodium starch glycolate and alginic acid.

Tablet binders that can be included are acacia, methylcellulose, sodium carboxymethylcellulose, polyvinylpyrrolidone (Povidone), hydroxypropyl methylcellulose, sucrose, starch and ethylcellulose.

Lubricants that can be used include magnesium stearate or other metallic stearates, stearic acid, silicone fluid, talc, waxes, oils and colloidal silica.

Flavoring agents such as peppermint, oil of wintergreen, cherry flavoring or the like can also be used. It may also be desirable to add a coloring agent to make the dosage form more aesthetic in appearance or to help identify the product.

For oral or parenteral administration, compounds of the present invention (e.g. compounds of Formula I, compounds of Formula A-I, compounds of Formula II, compounds of Formula A-II) preferably a compound of Formula A-I or Formula A-II, in conjunction with a β-lactam antibiotic, can be mixed with conventional pharmaceutical carriers and excipients and used in the form of tablets, capsules, elixirs, suspensions, syrups, wafers and the like. The compositions comprising a compound of this invention may contain from about 0.1% to about 99% by weight of the active compound, such as from about 10% to about 30%.

For oral use, solid formulations such as tablets and capsules are useful. Sustained release or enterically coated preparations may also be devised. For pediatric and geriatric applications, one embodiment provides suspensions, syrups and chewable tablets. For oral administration, the pharmaceutical compositions are in the form of, for example, a tablet, capsule, suspension or liquid.

The pharmaceutical compositions may be made in the form of a dosage unit containing a therapeutically-effective amount of the active ingredient. Examples of such dosage units are tablets and capsules. For therapeutic purposes, the tablets and capsules which can contain, in addition to the active ingredient, conventional carriers such as binding agents, for example, acacia gum, gelatin, polyvinylpyrrolidone, sorbitol, or tragacanth; fillers, for example, calcium phosphate, glycine, lactose, maize-starch, sorbitol, or sucrose; lubricants, for example, magnesium stearate, polyethylene glycol, silica, or talc; disintegrants, for example, potato starch, flavoring or coloring agents, or acceptable wetting agents. Oral liquid preparations generally are in the form of aqueous or oily solutions, suspensions, emulsions, syrups or elixirs, preparations of the invention may contain conventional additives such as suspending agents, emulsifying agents, non-aqueous agents, preservatives, coloring agents and flavoring agents. Non-limiting examples of additives for liquid preparations include acacia, almond oil, ethyl alcohol, fractionated coconut oil, gelatin, glucose syrup, glycerin, hydrogenated edible fats, lecithin, methyl cellulose, methyl or propylpara-hydroxybenzoate, propylene glycol, sorbitol, or sorbic acid.

For intravenous (IV) use, the pharmaceutical composition (e.g. compounds of Formula I, compounds of Formula A-I, compounds of Formula II, compounds of Formula A-II) preferably a compound of Formula A-I or Formula A-II, in conjunction with a β-lactam antibiotic, can be dissolved or suspended in any of the commonly used intravenous fluids and administered by infusion. Intravenous fluids include, without limitation, physiological saline or Ringer's solution. Intravenous administration may be accomplished by using, without limitation, syringe, mini-pump or intravenous line.

Pharmaceutical compositions of this invention (e.g. compounds of Formula I, compounds of Formula A-I, compounds of Formula II, compounds of Formula A-II) preferably a compound of Formula A-I or Formula A-II, for parenteral injection comprise pharmaceutically-acceptable aqueous or non-aqueous solutions, dispersions, suspensions or emulsions as well as sterile powders for reconstitution into sterile injectable solutions or dispersions just prior to use. Examples of suitable aqueous and non-aqueous carriers, diluents, solvents or vehicles include water, ethanol, benzyl alcohol, polyols (such as glycerol, propylene glycol, and polyethylene glycol), and suitable mixtures thereof, vegetable oils (such as corn oil or olive oil), and injectable organic esters such as ethyl oleate. Proper fluidity can be maintained, for example, by the use of coating materials such as lecithin, by the maintenance of the required particle size in the case of dispersions, and by the use of surfactants. The compositions can include various buffers.

These compositions may also contain adjuvants such as preservatives, wetting agents, emulsifying agents, and dispersing agents. They may also contain taggants or other anti-counterfeiting agents, which are well known in the art. Prevention of the action of microorganisms may be ensured by the inclusion of various antibacterial and antifungal agents, for example, paraben, chlorobutanol, and phenol sorbic acid. It may also be desirable to include isotonic agents such as sugars and sodium chloride. Prolonged absorption of the injectable pharmaceutical form may be brought about by the inclusion of agents which delay absorption, such as aluminum monostearate and gelatin.

Injectable depot forms can be made by forming microencapsulating matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations can also be prepared by entrapping the drug in liposomes or microemulsions, which are compatible with body tissues.

The injectable formulations can be sterilized, for example, by filtration through a bacterial-retaining filter, or by incorporating sterilizing agents in the form of sterile solid compositions, which can be dissolved or dispersed in sterile water or other sterile injectable medium just prior to use.

Solid dosage forms for oral administration include capsules, tablets, pills, powders, and granules. Such forms may include forms that dissolve or disintegrate quickly in the oral environment. In such solid dosage forms, the active compound preferably a compound of Formula A-I or Formula A-II in conjunction with a β-lactam antibiotic, can be mixed with at least one inert, pharmaceutically-acceptable excipient or carrier. Suitable excipients include, for example, (a) fillers or extenders such as starches, lactose, sucrose, glucose, mannitol, and silicic acid; (b) binders such as cellulose and cellulose derivatives (such as hydroxypropylmethylcellulose, hydroxypropylcellulose, and carboxymethylcellulose), alginates, gelatin, polyvinylpyrrolidone, sucrose, and acacia; (c) humectants such as glycerol; (d) disintegrating agents such as sodium starch glycolate, croscarmellose, agar-agar, calcium carbonate, potato or tapioca starch, alginic acid, certain silicates, and sodium carbonate; (e) solution retarding agents such as paraffin; (f) absorption accelerators such as quaternary ammonium compounds; (g) wetting agents, such as cetyl alcohol and glycerol monostearate, fatty acid esters of sorbitan, poloxamers, and polyethylene glycols; (h) absorbents such as kaolin and bentonite clay; (i) lubricants such as talc, calcium stearate, magnesium stearate, solid polyethylene glycols, sodium lauryl sulfate, and mixtures thereof; and (j) glidants such as talc, and silicone dioxide. Other suitable excipients include, for example, sodium citrate or dicalcium phosphate. The dosage forms may also comprise buffering agents.

Solid dosage forms, including those of tablets, dragees, capsules, pills, and granules, can be prepared with coatings and shells such as functional and aesthetic enteric coatings and other coatings well known in the pharmaceutical formulating art. They may optionally contain opacifying agents and colorants. They may also be in a form capable of controlled or sustained release. Examples of embedding compositions that can be used for such purposes include polymeric substances and waxes.

The pharmaceutical compositions can be delivered using controlled (e.g., capsules) or sustained release (e.g., bioerodable matrices) delivery systems. Exemplary delayed release delivery systems for drug delivery that are suitable for administering the pharmaceutical compositions are described in U.S. Pat. No. 4,452,775 (issued to Kent), U.S. Pat. No. 5,039,660 (issued to Leonard), and U.S. Pat. No. 3,854,480 (issued to Zaffaroni).

In some cases, in order to prolong the effect of the drug, it may be desirable to slow the absorption of the drug following subcutaneous or intramuscular injection. This may be accomplished by the use of a liquid suspension of crystalline or amorphous material with poor water solubility. Amorphous material may be used alone or together with stabilizers as necessary. The rate of absorption of the drug then depends upon its rate of dissolution, which in turn, may depend upon crystal size and crystalline form.

Alternatively, delayed absorption of a parenterally administered drug form can be accomplished by dissolving or suspending the drug in an oil vehicle.

For intramuscular preparations, a sterile formulation of compounds, preferably a compound of Formula A-I or Formula A-II in conjunction with a β-lactam antibiotic, or suitable soluble salt forms thereof, for example hydrochloride salts, can be dissolved and administered in a pharmaceutical diluent such as Water-for-Injection (WFI), physiological saline or 5% glucose. A suitable insoluble form of the compound may be prepared and administered as a suspension in an aqueous base or a pharmaceutically acceptable oil base, e.g., an ester of a long chain fatty acid such as ethyl oleate.

A dose of an intravenous, intramuscular, or parental formulation of compounds, preferably a compound of Formula A-I or Formula A-II in conjunction with a β-lactam antibiotic, may be administered as a bolus or by slow infusion. A bolus is a dose that is administered in less than 30 minutes. In one embodiment, a bolus is administered in less than 15 or less than 10 minutes. In another embodiment, a bolus is administered in less than 5 minutes. In yet another embodiment, a bolus is administered in one minute or less. An infusion is a dose that is administered at a rate of 30 minutes or greater. In one embodiment, the infusion is one hour or greater. In another embodiment, the infusion is substantially constant.

For topical use the pharmaceutical compositions, preferably a compound of Formula A-I or Formula A-II in conjunction with a β-lactam antibiotic, can also be prepared in suitable forms to be applied to the skin, or mucus membranes of the nose and throat, and can take the form of creams, ointments, liquid sprays or inhalants, lozenges, or throat paints. Such topical formulations further can include chemical compounds such as dimethylsulfoxide (DMSO) to facilitate surface penetration of the active ingredient.

For application to the eyes or ears, the pharmaceutical composition can be presented in liquid or semi-liquid form formulated in hydrophobic or hydrophilic bases as ointments, creams, lotions, paints or powders.

For rectal administration, the pharmaceutical compositions, preferably a compound of Formula A-I or Formula A-II in conjunction with a β-lactam antibiotic, can be administered in the form of suppositories admixed with conventional carriers such as cocoa butter, polyethylene glycol or a suppository wax or other glyceride that are solid at room temperature but liquid at body temperature and therefore melt in the rectum or vaginal cavity and release the active compound.

Alternatively, the pharmaceutical compositions can be in powder form for reconstitution in the appropriate pharmaceutically acceptable carrier at the time of delivery. In another embodiment, the unit dosage form of compounds, preferably a compound of Formula A-I or Formula A-II in conjunction with a β-lactam antibiotic, can be a solution of one or more compounds, or salts thereof, in a suitable diluent, in sterile hermetically sealed ampoules or sterile syringes. The concentration of the compounds, preferably a compound of Formula A-I or Formula A-II in conjunction with a β-lactam antibiotic, in the unit dosage may vary, e.g. from about 1 percent to about 50 percent, depending on the compound used and its solubility and the dose desired by the physician. If the compositions contain dosage units, each dosage unit can contain from 1-500 mg of the active material. For adult human treatment, the dosage employed can range from 5 mg to 10 g, per day, depending on the route and frequency of administration.

The pharmaceutical compositions disclosed herein can be placed in a pharmaceutically acceptable carrier and are delivered to a recipient subject (e.g., a human) in accordance with known methods of drug delivery. In general, the methods of delivering the pharmaceutical compositions in vivo utilize art-recognized protocols for delivering the agent with the only substantial procedural modification being the substitution of the compounds of the present invention for the drugs in the art-recognized protocols. Likewise, methods for using the claimed compositions for treating cells in culture, for example, to eliminate or reduce the level of bacterial contamination of a cell culture, utilize art-recognized protocols for treating cell cultures with antibacterial agent(s) with the only substantial procedural modification being the substitution of the compounds of the present invention, preferably in combination with a β-lactam antibiotic for the drugs in the art-recognized protocols.

Exemplary procedures for delivering an antibacterial agent are described in U.S. Pat. Nos. 6,468,967; 6,852,689; and 5,041,567, issued to Rogers and in PCT patent application number EP94/02552 (publication no. WO 95/05384), the disclosures of which are incorporated herein by reference in their entirety. In one embodiment, one or more compounds of the invention, preferably a compound of Formula A-I or Formula A-II in conjunction with a β-lactam antibiotic, or pharmaceutical compositions thereof are administered orally, rectally or via injection (intravenous, intramuscular or subcutaneous). In another embodiment, one or more compounds of the invention, preferably a compound of Formula A-I or Formula A-II in conjunction with a β-lactam antibiotic, or pharmaceutical compositions thereof are administered orally, rectally or via injection (intravenous, intramuscular or subcutaneous) to treat an infection caused by β-lactam resistant bacteria. In another embodiment, one or more compounds of the invention, preferably a compound of Formula A-I or Formula A-II in conjunction with a β-lactam antibiotic, or pharmaceutical compositions thereof are administered orally to treat an infection caused by β-lactamase producing bacteria.

As used herein, the phrases “therapeutically-effective dose” and “therapeutically-effective amount” refer to an amount of a compound that prevents the onset, alleviates the symptoms, stops the progression of a bacterial infection, or results in another desired biological outcome such as, e.g., improved clinical signs or reduced/elevated levels of lymphocytes and/or antibodies.

The term “treating” or “treatment” is defined as administering, to a subject, a therapeutically-effective amount of one or more compounds both to prevent the occurrence of an infection and to control or eliminate an infection. Those in need of treatment may include individuals already having a particular medical disease as well as those at risk for the disease (i.e., those who are likely to ultimately acquire the disorder).

The term “subject,” as used herein, refers to a mammal, a plant, a lower animal, or a cell culture. In one embodiment, a subject is a human or other animal patient in need of antibacterial treatment.

The term “administering “or “administration” and the like, refers to providing the compound of the invention (e.g. compounds of Formula I, compounds of Formula A-I, compounds of Formula II, compounds of Formula A-II) to the subject in need of treatment. Preferably the subject is a mammal, more preferably a human. The present invention comprises administering the compound of the invention (e.g. compounds of Formula I, compounds of Formula A-I, compounds of Formula II, compounds of Formula A-II) in conjunction with a β-lactam antiobiotic. When a compound of the invention (e.g. compounds of Formula I, compounds of Formula A-I, compounds of Formula II, compounds of Formula A-II) is administered in conjunction with a β-lactam antiobiotic, the compound of the invention (e.g. compounds of Formula I, compounds of Formula A-I, compounds of Formula II, compounds of Formula A-II) and the β-lactam antiobiotic can be administered at the same time or different times. When the compounds of the invention (e.g. compounds of Formula I, compounds of Formula A-I, compounds of Formula II, compounds of Formula A-II) and the β-lactam antiobiotic are administered at the same time, they can be administered as a single composition or pharmaceutical composition or they can be administered separately. It is understood that when a compound of the invention (e.g. compounds of Formula I, compounds of Formula A-I, compounds of Formula II, compounds of Formula A-II) is administered in conjunction with a β-lactam antibiotic, that the active agents can be administered in a single combination or in multiple combinations. For example, when administered by IV, the compound of the invention (e.g. compounds of Formula I, compounds of Formula A-I, compounds of Formula II, compounds of Formula A-II) can be dissolved or suspended in any of the commonly used intravenous fluids and administered by infusion, then a β-lactam antibiotic can be dissolved or suspended in any of the commonly used intravenous fluids and administered by infusion. Conversely the β-lactam antibiotic can be dissolved or suspended in any of the commonly used intravenous fluids and administered by infusion, then a compound of Formula I can be dissolved or suspended in any of the commonly used intravenous fluids and administered by infusion. Alternatively, a pharmaceutical composition comprising a compound of the invention (e.g. compounds of Formula I, compounds of Formula A-I, compounds of Formula II, compounds of Formula A-II) and a β-lactam antibiotic can be dissolved or suspended in any of the commonly used intravenous fluids and administered by infusion.

In one embodiment of the invention, is provided a method of treating or preventing a bacterial infection comprising administering to a subject in need thereof a therapeutically-effective amount of the pharmaceutical composition comprising a compound of the invention (e.g. compounds of Formula I, compounds of Formula A-I, compounds of Formula II, compounds of Formula A-II) and a β-lactam antibiotic. In one embodiment of the invention, is provided a method of treating or preventing a bacterial infection comprising administering to a subject in need thereof a therapeutically-effective amount of the pharmaceutical composition comprising a compound of Formula I, and a β-lactam antibiotic. In one embodiment of the invention, is provided a method of treating or preventing a bacterial infection comprising administering to a subject in need thereof a therapeutically-effective amount of the pharmaceutical composition comprising a compound of Formula A-I, and a β-lactam antibiotic. In one embodiment of the invention, is provided a method of treating or preventing a bacterial infection comprising administering to a subject in need thereof a therapeutically-effective amount of the pharmaceutical composition comprising a compound of Formula II, and a β-lactam antibiotic. In one embodiment of the invention, is provided a method of treating or preventing a bacterial infection comprising administering to a subject in need thereof a therapeutically-effective amount of the pharmaceutical composition comprising a compound of Formula A-II, and a β-lactam antibiotic.

In one embodiment of the invention, is provided a method of treating or preventing a bacterial infection comprising administering to a subject in need thereof, a therapeutically-effective amount of a β-lactam antibiotic in conjunction with a compound of the invention (e.g. compounds of Formula I, compounds of Formula A-I, compounds of Formula II, compounds of Formula A-II). In one embodiment of the invention, is provided a method of treating or preventing a bacterial infection comprising administering to a subject in need thereof, a therapeutically-effective amount of a β-lactam antibiotic in conjunction with a compound of Formula I. In one embodiment of the invention, is provided a method of treating or preventing a bacterial infection comprising administering to a subject in need thereof, a therapeutically-effective amount of a β-lactam antibiotic in conjunction with a compound of Formula A-I. In one embodiment of the invention, is provided a method of treating or preventing a bacterial infection comprising administering to a subject in need thereof, a therapeutically-effective amount of a β-lactam antibiotic in conjunction with a compound of Formula II. In one embodiment of the invention, is provided a method of treating or preventing a bacterial infection comprising administering to a subject in need thereof, a therapeutically-effective amount of a β-lactam antibiotic in conjunction with a compound of Formula A-II. In one embodiment of the invention, is provided a method of treating or preventing a bacterial infection comprising administering to a subject in need thereof, a therapeutically-effective amount of a β-lactam antibiotic in conjunction with a compound of Table I. In one embodiment of the invention, is provided a method of treating or preventing a bacterial infection comprising administering to a subject in need thereof, a therapeutically-effective amount of a β-lactam antibiotic in conjunction with a compound of Formula

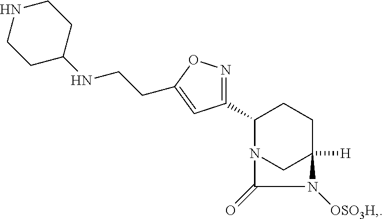

In one embodiment of the invention, is provided a method of treating or preventing a bacterial infection comprising administering to a subject in need thereof, a therapeutically-effective amount of a β-lactam antibiotic in conjunction with a compound of Formula

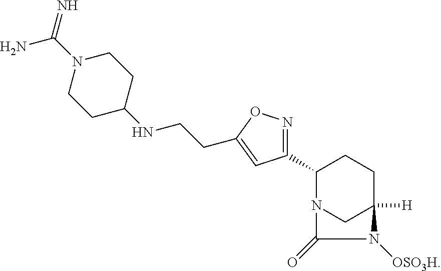

In one embodiment of the invention, is provided a method of treating or preventing a bacterial infection in a subject comprising the steps of
a. administering to the subject a compound of the invention; and
b. administering to the subject a therapeutically-effective amount of a β-lactam antibiotic.

In one embodiment the compound in step a is a compound of Formula I. In one embodiment the compound in step a is a compound of Formula A-I. In one embodiment the compound in step a is a compound of Formula II. In one embodiment the compound in step a is a compound of Formula A-II. In one embodiment the compound in step a is a compound of Table I. In one embodiment the compound in step a is a compound of Formula A-II. In one embodiment the compound in step a is a compound of Formula

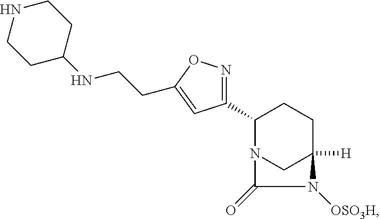

In one embodiment the compound in step a is a compound of Formula

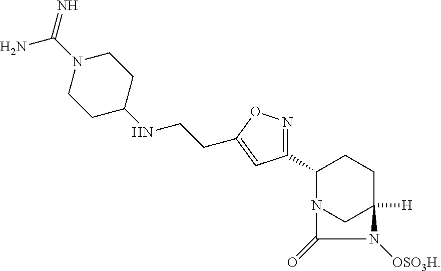

In one embodiment, the β-lactam antibiotic in step b is Ceftolozane or Ceftazidime. In one embodiment the compound in step a is a compound of Formula

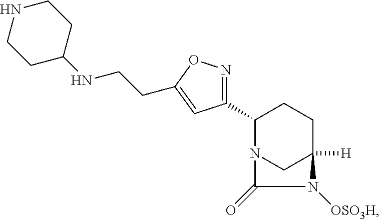

and the β-lactam antibiotic in step b is Ceftolozane. In one embodiment the compound in step a is a compound of Formula

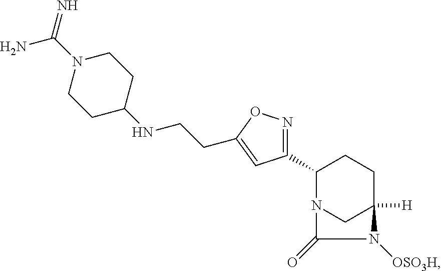

and the β-lactam antibiotic in step b is Ceftolozane.

In one embodiment of the invention, is provided a method of treating or preventing a bacterial infection in a subject comprising the steps of
a. administering to the subject a therapeutically-effective amount of a β-lactam antibiotic; and
b. administering to the subject a compound of the invention.

In one embodiment the compound in step b is a compound of Formula I. In one embodiment the compound in step b is a compound of Formula A-I. In one embodiment the compound in step b is a compound of Formula II. In one embodiment the compound in step b is a compound of Formula A-II. In one embodiment the compound in step b is a compound of Table I. In one embodiment the compound in step b is a compound of Formula

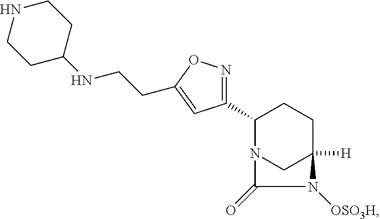

In one embodiment the compound in step b is a compound of Formula

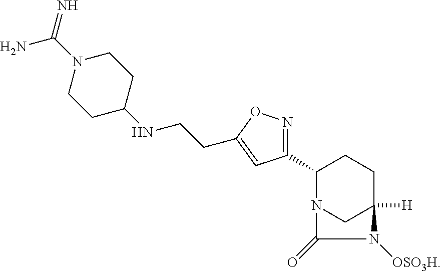

In one embodiment, the β-lactam antibiotic in step a is Ceftolozane or Ceftazidime. In one embodiment the compound in step b is a compound of Formula

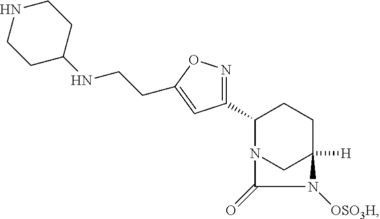

and the β-lactam antibiotic in step a is Ceftolozane. In one embodiment the compound in step a is b compound of Formula

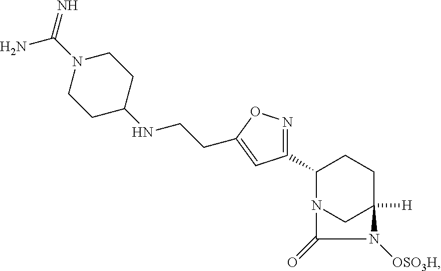

and the β-lactam antibiotic in step a is Ceftolozane. In one embodiment, the invention provides a method for treating an infection in a subject by administering a therapeutically-effective amount of one or more compounds of the invention (e.g. compounds of Formula I, compounds of Formula A-I, compounds of Formula II, compounds of Formula A-II), preferably a compound of Formula A-I or Formula A-II in conjunction with a β-lactam antibiotic, or compositions thereof. In one embodiment, the method comprises administering to a subject in need thereof a pharmaceutical composition comprising at least one of the compounds described herein, preferably a compound of Formula A-I or Formula A-II in conjunction with a β-lactam antibiotic. In one embodiment the compound is of Formula

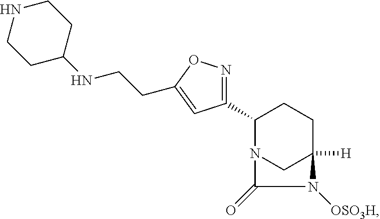

in conjunction with a β-lactam antibiotic, preferably Ceftolozane or Ceftazidime, or compositions thereof. In one embodiment the compound is of Formula

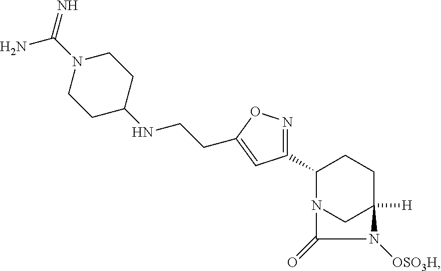

in conjunction with a β-lactam antibiotic, preferably Ceftolozane or Ceftazidime, or compositions thereof. In one embodiment, the pharmaceutical composition can comprise any one of the compounds described herein as the sole active compound or in combination with another compound, composition, or biological material. The compound may be administered orally, parenterally, by inhalation, topically, rectally, nasally, buccally, vaginally, or by an implanted reservoir, external pump or catheter. The compound may be prepared for opthalmic or aerosolized uses. The compounds of the present invention can be administered as an aerosol for the treatment of pneumonia or other lung-based infections. In one embodiment, the aerosol delivery vehicle is an anhydrous or dry powder inhaler. One or more compounds of the invention (e.g. compounds of Formula I, compounds of Formula A-I, compounds of Formula II, compounds of Formula A-II), preferably a compound of Formula A-I or Formula A-II in conjunction with a β-lactam antibiotic, or pharmaceutical compositions thereof also may be directly injected or administered into an abscess, ventricle or joint. Parenteral administration includes subcutaneous, intravenous, intramuscular, intra-articular, intra-synovial, cisternal, intrathecal, intrahepatic, intralesional and intracranial injection or infusion. In one embodiment, one or more compounds of the invention (e.g. compounds of Formula I, compounds of Formula A-I, compounds of Formula II, compounds of Formula A-II), preferably a compound of Formula A-I or Formula A-II in conjunction with a β-lactam antibiotic, are administered intravenously, subcutaneously or orally. In one embodiment for administering one or more compounds according to the invention (e.g. compounds of Formula I, compounds of Formula A-I, compounds of Formula II, compounds of Formula A-II), preferably a compound of Formula A-I or Formula A-II in conjunction with a β-lactam antibiotic to a cell culture, the one or more compounds may be administered in a nutrient medium.

In one embodiment, one or more compounds according to the invention (e.g. compounds of Formula I, compounds of Formula A-I, compounds of Formula II, compounds of Formula A-II), preferably a compound of Formula A-I or A-II in conjunction with a β-lactam antibiotic, may be used to treat a subject having a bacterial infection in which the infection is caused or exacerbated by any type of bacteria, such as Gram-negative bacteria. In one aspect of the invention, the bacterial infection is caused by β-lactam resistant bacteria. In one aspect the bacterial infection is caused by β-lactamase producing bacteria. In another aspect the bacterial infection is caused by class A, class C or class D β-lactamase producing bacteria. In another aspect the bacterial infection is caused by class A β-lactamase producing bacteria. In another aspect the infection is caused by class C β-lactamase producing bacteria. In still another aspect the infection is caused by class D β-lactamase producing bacteria. In still another aspect the infection is caused by KPC β-lactamase producing bacteria. In still another aspect the infection is caused by OXA β-lactamase producing bacteria. In still another aspect, the bacterial infection is caused by a bacteria that produces multiple β-lactamases. Bacteria that produce multiple β-lactamases may produce β-lactamases of the same class or of different classes (e.g. class A and class A or class A and class C or class A and class D etc).

Representative Gram-negative pathogens known to express β-lactamases include, but are not limited to Acinetobacter spp. (including Acinetobacter baumannii), Citrobacter spp., Escherichia spp. (including Escherichia coli), Haemophilus influenzae, Morganella morganii, Pseudomonas aeruginosa, Klebsiella spp. (including Klebsiella pneumoniae), Enterobacter spp. (including Enterobacter cloacae and Enterobacter aerogenes), Pasteurella spp., Proteus spp. (including Proteus mirabilis), Serratia spp. (including Serratia marcescens), and Providencia spp. Bacterial infections can be caused or exacerbated by Gram-negative bacteria including strains which express β-lactamases that may confer resistance to penicillins, cephalosporins, monobactams and/or carbapenems. The co-administration of a novel BLI that inhibits these β-lactamases with a β-lactam antibiotic could be used to treat infections caused by β-lactam resistant bacteria.

In one aspect of the invention the infection is caused by a β-lactamase producing bacteria selected from Acinetobacter spp, Citrobacter spp, Escherichia coli, Enterobacter cloacae), Haemophilus influenzae, Pseudomonas aeruginosa, Proteus mirabilis, Serratia marcescens, and Klebsiella pneumoniae,

β-Lactam antibiotics that may be administered concurrently with compounds of the invention (e.g. compounds of Formula I, compounds of Formula A-I, compounds of Formula II, compounds of Formula A-II) include, but are not limited to cephalosporin, carbapenem, monobactam, penem and penicillin classes of antibiotics.

In one embodiment of the invention, the β-lactam antibiotic is a cephalosporin. Examples of cephalosporins include, but are not limited to, Cefacetrile (cephacetrile), Cefadroxil (cefadroxyl), Cefalexin (cephalexin), Cefaloglycin (cephaloglycin), Cefalonium (cephalonium), Cefaloridine (cephaloradine), Cefalotin (cephalothin), Cefapirin (cephapirin), Cefatrizine, Cefazaflur, Cefazedone, Cefazolin (cephazolin), Cefradine (cephradine), Cefroxadine, Ceftezole, Cefaclor, Cefamandole, Cefmetazole, Cefonicid, Cefotetan, Cefoxitin, Cefprozil (cefproxil), Cefuroxime, Cefuzonam, Cefcapene, Cefdaloxime, Cefdinir, Cefditoren, Cefetamet, Cefixime, Cefmenoxime, Cefodizime, Cefotaxime, Cefpimizole, Cefpodoxime, Cefteram, Ceftibuten, Ceftiofur, Ceftiolene, Ceftizoxime, Ceftriaxone, Cefoperazone, Ceftazidime, Cefclidine, Cefepime, Cefluprenam, Cefoselis, Cefozopran, Cefpirome, Cefquinome, Cefaclomezine, Cefaloram, Cefaparole, Cefcanel, Cefedrolor, Cefempidone, Cefetrizole, Cefivitril, Cefmatilen, Cefmepidium, Cefovecin, Cefoxazole, Cefrotil, Cefsumide, Ceftaroline, Ceftioxide, Cefuracetime, cefbuperazone, cefminox, ceforanide, cefotiam, cefpiramide, cefsulodin, ceftobiprole latamoxef, loracarbef and Ceftolozane. In one embodiment the cephalosporin is Ceftolozane or Ceftazidime.

In one embodiment of the invention, the β-lactam antibiotic is a carbapenen. Examples of carbapenem antibiotics include, but are not limited to, Imipenem, Imipenem/Cilastatin, Biapenem, Doripenem, Meropenem, Ertapenem and Panipenem. In one embodiment the Carbapenem is Imipenem/Cilastatin or Meropenem.

In one embodiment of the invention, the β-lactam antibiotic is a monobactam. Examples of monobactam antibiotics include, but are not limited to Aztreonam, Tigemonam, Carumonam, BAL30072 and Nocardicin A.

In one embodiment of the invention, the β-lactam antibiotic is a penem. In one embodiment of the invention, the β-lactam antibiotic is a penicillin. Examples of penicillin antibiotics include, but are not limited to Amoxicillin, Ampicillin, Azlocillin, Mezlocillin, Apalcillin, Hetacillin, Becampicillin, Carbenicillin, Sulbenicillin, Ticarcillin, Piperacillin, Azlocillin, Mecillinam, Pivmecillinam, Methicillin, Ciclacillin, Talampicillin, Aspoxicillin, Oxacillin, Cloxacillin, Dicloxacillin, Flucloxacillin, Nafcillin and Pivampicillin.

The pharmaceutical compositions, preferably a compound of the invention (e.g.

compounds of Formula I, compounds of Formula A-I, compounds of Formula II, compounds of Formula A-II) in conjunction with a β-lactam antibiotic, can be used to treat a bacterial infection of any organ or tissue in the body caused by β-lactam resistant bacteria, preferably, Gram-negative β-lactam resistant bacteria. These organs or tissue include, without limitation, skeletal muscle, skin, bloodstream, kidneys, heart, lung and bone. For example, a pharmaceutical composition comprising at least one compound of the invention (e.g. compounds of Formula I, compounds of Formula A-I, compounds of Formula II, compounds of Formula A-II), preferably a compound of Formula A-I or Formula A-II in conjunction with a β-lactam antibiotic, can be administered to a subject to treat, without limitation, skin and soft tissue infections (e.g., complex skin infections), bacteremia, intra-abdominal infections and urinary tract infections (e.g., cUTI). In addition, a compound of the invention (e.g. compounds of Formula I, compounds of Formula A-I, compounds of Formula II, compounds of Formula A-II) may be used to treat community acquired respiratory infections, including, without limitation, otitis media, sinusitis, chronic bronchitis and pneumonia (including community-acquired pneumonia, hospital-acquired pneumonia and ventilator associated pneumonia), including pneumonia caused by drug-resistant Pseudomonas aeruginosa. At least one compound of the invention (e.g. compounds of Formula I, compounds of Formula A-I, compounds of Formula II, compounds of Formula A-II), preferably a compound of Formula A-I or Formula A-II in conjunction with a β-lactam antibiotic, can be administered to a subject to treat mixed infections that comprise different types of Gram-negative bacteria, or which comprise both Gram-positive and Gram-negative bacteria. These types of infections include intra-abdominal infections and obstetrical/gynecological infections. At least one compound of the invention (e.g. compounds of Formula I, compounds of Formula A-I, compounds of Formula II, compounds of Formula A-II), preferably a compound of Formula A-I or Formula A-II in conjunction with a β-lactam antibiotic, may also be administered to a subject to treat an infection including, without limitation, endocarditis, nephritis, septic arthritis, intra-abdominal sepsis, bone and joint infections and osteomyelitis. At least one compound of the invention (e.g. compounds of Formula I, compounds of Formula A-I, compounds of Formula II, compounds of Formula A-II), preferably compound of Formula A-I or Formula A-II in conjunction with a β-lactam antibiotic, or pharmaceutical compositions thereof, may also be directly injected or administered into an abscess, ventricle or joint. Pharmaceutical compositions of the invention (e.g. compounds of Formula I, compounds of Formula A-I, compounds of Formula II, compounds of Formula A-II), preferably compound of Formula A-I or Formula A-II in conjunction with a β-lactam antibiotic, may be administered as an aerosol for the treatment of pneumonia or other lung-based infections. In one embodiment, the aerosol delivery vehicle is an anhydrous, liquid or dry powder inhaler.

Actual dosage levels of active ingredients in the pharmaceutical compositions of one or more compounds according to the invention (e.g. compounds of Formula I, compounds of Formula A-I, compounds of Formula II, compounds of Formula A-II), preferably a compound of Formula A-I or Formula A-II in conjunction with a β-lactam antibiotic, may be varied so as to obtain a therapeutically-effective amount of the active compound(s) to achieve the desired therapeutic response for a particular patient, compositions, and mode of administration. The effective amount can be determined as described herein. The selected dosage level will depend upon the activity of the particular compound, the route of administration, the severity of the condition being treated, and the condition and prior medical history of the patient being treated. However, it is within the skill of the art to start doses of the compound at levels lower than required to achieve the desired therapeutic effect and to gradually increase the dosage until the desired effect is achieved. In one embodiment, the data obtained from the assays can be used in formulating a range of dosage for use in humans. It will be understood by one of skill in the art that the when the composition comprises a compound of the invention (e.g. compounds of Formula I, compounds of Formula A-I, compounds of Formula II, compounds of Formula A-II) and a β-lactam antibiotic, both the compound of the invention (e.g. compounds of Formula I, compounds of Formula A-I, compounds of Formula II, compounds of Formula A-II) and the β-lactam antibiotic are active compounds.

The method comprises administering to the subject an effective dose of one or more compounds of the invention (e.g. compounds of Formula I, compounds of Formula A-I, compounds of Formula II, compounds of Formula A-II), preferably in conjunction with a β lactam antibiotic. An effective dose of a compound of the invention (e.g. compounds of Formula I, compounds of Formula A-I, compounds of Formula II, compounds of Formula A-II) is generally between 125 mg/day to 2000 mg/day. In one embodiment, an effective dose is from about 0.1 to about 100 mg/kg of one or more compounds of the invention (e.g. compounds of Formula I, compounds of Formula A-I, compounds of Formula II, compounds of Formula A-II) or pharmaceutically acceptable salts thereof. In one embodiment, the dose is from about 0.1 to about 50 mg/kg of one or more compounds of the invention (e.g. compounds of Formula I, compounds of Formula A-I, compounds of Formula II, compounds of Formula A-II) or pharmaceutically acceptable salts thereof. In another embodiment, the dose is from about 1 to about 25 mg/kg of one or more compounds of the invention (e.g. compounds of Formula I, compounds of Formula A-I, compounds of Formula II, compounds of Formula A-II) or pharmaceutically acceptable salts thereof. In another embodiment, the dose is from about 1 to about 12 mg/kg of one or more compounds of the invention (e.g. compounds of Formula I, compounds of Formula A-I, compounds of Formula II, compounds of Formula A-II). In another embodiment, the dose is about 1, 2, 3, 4, 5, 6, 7, 8, 9, 10, 11, or 12 mg/kg of one or more compounds of the invention (e.g. compounds of Formula I, compounds of Formula A-I, compounds of Formula II, compounds of Formula A-II). In another embodiment, the compounds of the invention (e.g. compounds of Formula I, compounds of Formula A-I, compounds of Formula II, compounds of Formula A-II) are administered to a human at a dose of 100 mg to 1000 mg per dose up to four times per day. In another embodiment, the compounds of the invention (e.g. compounds of Formula I, compounds of Formula A-I, compounds of Formula II, compounds of Formula A-II) are administered to a human at a dose of 125 mg to 750 mg per dose up to four times per day. In another embodiment, the compounds of the invention (e.g. compounds of Formula I, compounds of Formula A-I, compounds of Formula II, compounds of Formula A-II) are administered to a human at a dose of 250 mg to 500 mg per dose up to four times a day. An effective dose for cell culture is usually between about 0.1 and about 1000 μg/mL. In one embodiment, the effect dose for cell culture is between about 0.1 and about 200 μg/mL.

In one embodiment, aβ-lactam antibiotic and a compound of the invention (e.g.

compounds of Formula I, compounds of Formula A-I, compounds of Formula II, compounds of Formula A-II) are administered in ratio of 1:4 to 8:1 antibiotic: compound of the invention (e.g. compounds of Formula I, compounds of Formula A-I, compounds of Formula II, compounds of Formula A-II). In one embodiment the ratio is 1:4. In another embodiment the ratio is 3:4. In another embodiment the ratio is 5:4. In another embodiment the ratio is 7:4. In another embodiment the ratio is 1:2. In another embodiment the ratio is 3:2. In another embodiment the ratio is 5:2. In another embodiment the ratio is 7:2. In another embodiment the ratio is 1:3. In another embodiment the ratio is 2:3. In another embodiment the ratio is 4:3. In another embodiment the ratio is 5:3. In another embodiment the ratio is 7:3. In another embodiment the ratio is 1:2. In another embodiment the ratio is 3:2. In another embodiment the ratio is 5:2. In another embodiment the ratio is 7:2. In another embodiment the ratio is 1:1. In another embodiment the ratio is 2:1. In another embodiment the ratio is 3:1. In another embodiment the ratio is 4:1. In another embodiment the ratio is 5:1. In another embodiment the ratio is 6:1. In another embodiment the ratio is 7:1. In another embodiment the ratio is 8:1. It will be understood by one of skill in the art that the β-lactam antibiotic and compound of the invention (e.g. compounds of Formula I, compounds of Formula A-I, compounds of Formula II, compounds of Formula A-II) can be administered within the range of ratios provided regardless of the method of drug delivery. It will also be understood by one of skill in the art that the β-lactam antibiotic and compound of the invention (e.g.

compounds of Formula I, compounds of Formula A-I, compounds of Formula II, compounds of Formula A-II) can be administered within the range of ratios provided together, for example, in a pharmaceutical composition, or sequentially, i.e. the β-lactam antibiotic is administered, followed by administration of a compound of the invention (e.g. compounds of Formula I, compounds of Formula A-I, compounds of Formula II, compounds of Formula A-II) or vice versa.

One or more compounds of the invention (e.g. compounds of Formula I, compounds of Formula A-I, compounds of Formula II, compounds of Formula A-II) may also be administered in the diet or feed of a patient or animal. If administered as part of a total dietary intake, the amount of compound employed can be less than 1% by weight of the diet, such as no more than 0.5% by weight. The diet for animals can be normal foodstuffs to which the compound can be added or it can be added to a premix.

One or more compounds of the invention (e.g. compounds of Formula I, compounds of Formula A-I, compounds of Formula II, compounds of Formula A-II), preferably a compound of Formula A-I or Formula A-II in conjunction with a β-lactam antibiotic, can be administered as a single daily dose or in multiple doses per day. In one embodiment, one or more compounds of the invention (e.g. compounds of Formula I, compounds of Formula A-I, compounds of Formula II, compounds of Formula A-II), preferably a compound of Formula A-I or Formula A-II in conjunction with a β-lactam antibiotic, is administered as a single dose per day. In another embodiment, one or more compounds of the invention (e.g. compounds of Formula I, compounds of Formula A-I, compounds of Formula II, compounds of Formula A-II), preferably a compound of Formula A-I of Formula A-II in conjunction with a β-lactam antibiotic is administered as two equal doses per day. In another embodiment, the compounds of the invention (e.g. compounds of Formula I, compounds of Formula A-I, compounds of Formula II, compounds of Formula A-II), preferably a compound of Formula A-I or Formula A-II in conjunction with a β-lactam antibiotic is administered in three equal doses per day. In another embodiment, the compounds of the invention (e.g. compounds of Formula I, compounds of Formula A-I, compounds of Formula II, compounds of Formula A-II), preferably a compound of Formula A-I or Formula A-II in conjunction with a β-lactam antibiotic is administered in four equal doses per day. The treatment regime may require administration over extended periods of time, e.g., for several days or for from two to four weeks. The amount per administered dose or the total amount administered will depend on such factors as the nature and severity of the infection, the age and general health of the patient, the tolerance of the patient to the compound of the invention and the β-lactam antibiotic and the microorganism or microorganisms involved in the infection. The treatment regimen for one type of infection may differ greatly from the treatment regimen of another infection. For example, one type of infection may require administration via intravenous administration once daily, while another infection may require a treatment regimen of multiple dosing orally.

One or more compounds of the invention (e.g. compounds of Formula I, compounds of Formula A-I, compounds of Formula II, compounds of Formula A-II), preferably a compound of Formula A-I or Formula A-II in conjunction with a β-lactam antibiotic, may be administered according to this method until the bacterial infection is eradicated or reduced. In one embodiment, one or more compounds of the invention (e.g. compounds of Formula I, compounds of Formula A-I, compounds of Formula II, compounds of Formula A-II), preferably a compound of Formula A-I or Formula A-II in conjunction with a β-lactam antibiotic, are administered for a period of time from 3 days to 6 months. In another embodiment, one or more compounds of the invention (e.g. compounds of Formula I, compounds of Formula A-I, compounds of Formula II, compounds of Formula A-II), preferably a compound of Formula A-I or Formula A-II in conjunction with a β-lactam antibiotic, are administered for 7 to 56 days. In another embodiment, one or more compounds of the invention (e.g. compounds of Formula I, compounds of Formula A-I, compounds of Formula II, compounds of Formula A-II), preferably a compound of Formula A-I or Formula A-II in conjunction with a β-lactam antibiotic, are administered for 7 to 28 days. In a further embodiment, one or more compounds of the invention (e.g. compounds of Formula I, compounds of Formula A-I, compounds of Formula II, compounds of Formula A-II), preferably a compound of Formula A-I or Formula A-II in conjunction with a β-lactam antibiotic, are administered for 7 to 14 days. Compounds of the present invention may be administered for a longer or shorter time period if it is so desired.

Other embodiments of the invention include:

A pharmaceutical composition comprising a compound of the invention (e.g. compounds of Formula I, compounds of Formula A-I, compounds of Formula II, compounds of Formula A-II), preferably a compound of Formula A-I or Formula A-II and at least 1 β-lactam antibiotic or a pharmaceutically acceptable salt thereof.

A pharmaceutical composition comprising a compound of the invention (e.g. compounds of Formula I, compounds of Formula A-I, compounds of Formula II, compounds of Formula A-II), preferably a compound of Formula A-I or Formula A-II and at least 1 cephalosporin antibiotic or a pharmaceutically acceptable salt thereof.

A pharmaceutical composition comprising a compound of the invention (e.g. compounds of Formula I, compounds of Formula A-I, compounds of Formula II, compounds of Formula A-II), preferably a compound of Formula A-I or Formula A-II and Ceftolozane or a pharmaceutically acceptable salt thereof.

A pharmaceutical composition comprising a compound of the invention (e.g. compounds of Formula I, compounds of Formula A-I, compounds of Formula II, compounds of Formula A-II), preferably a compound of Formula A-I or Formula A-II and at least 1 carbapenem antibiotic or a pharmaceutically acceptable salt thereof.

A pharmaceutical composition comprising a compound of the invention (e.g. compounds of Formula I, compounds of Formula A-I, compounds of Formula II, compounds of Formula A-II), preferably a compound of Formula A-I or Formula A-II and at least 1 monobactam antibiotic or a pharmaceutically acceptable salt thereof.

The embodiments described herein provide compounds of the invention (e.g. compounds of Formula I, compounds of Formula A-I, compounds of Formula II, compounds of Formula A-II), preferably a compound of Formula A-I or Formula A-II that are novel and active β-lactamase inhibitors. Other embodiments described herein provide novel compounds of the invention (e.g. compounds of Formula I, compounds of Formula A-I, compounds of Formula II, compounds of Formula A-II), preferably a compound of Formula A-I or Formula A-II in conjunction with β-lactam antibiotics for treatment of infections. Further embodiments described herein provide novel compounds of the invention (e.g. compounds of Formula I, compounds of Formula A-I, compounds of Formula II, compounds of Formula A-II), preferably a compound of Formula A-I or Formula A-II that show unexpected activity against β-lactamases that other compounds in the class do not have.

### Preparation of Compounds of the Invention

A compound of the invention (e.g. compounds of Formula I, compounds of Formula A-I, compounds of Formula II, compounds of Formula A-II) can be prepared by a variety of synthetic routes, including synthetic schemes described herein. These synthetic routes can be applied to large scale synthesis with appropriate adjustment of reaction sequence, reaction conditions, isolation/purification methods and choice of solvents which are environmentally friendly and cost-effective.

The following abbreviations have the following meanings unless otherwise indicated. Abbreviations not defined below have their generally accepted meaning.
Bn=benzyl
Boc=tert-butoxycarbonyl
Boc₂O=di-tert-butyldicarbonate
Burgess reagent=methyl N-triethylammoniumsulfonyl)carbamate
CDI=carbonyldiimidazole
CFU=colony-forming units
CLSI=Clinical Laboratory Standards Institute
cSSSI=complicated skin and skin structure infections
DBU=1,8-diazabicyclo[5.4.0]undec-7-ene
DCM=dichloromethane
DEAD=diethyl azodicarboxylate
DIAD=diisopropyl azodicarboxylate
DIPEA=diisopropylethylamine
DMF=N,N-dimethylformamide
DMAc=N,N-dimethylacetamide
DMSO=dimethyl sulfoxide
EDCI=1-ethyl-3-(3′-dimethylaminopropyl)carbodiimide
ELSD=evaporative light scattering detector
EtOAc=ethyl acetate
ESI-MS=electrospray ionization mass spectrometry
Fmoc=Fluorenylmethyloxycarbonyl
HAP=Hospital-Acquired Pneumonia
HATU=2-(7-aza-1H-benzotriazole-1-yl)-1,1,3,3-tetramethyluronium hexafluorophosphate
HCl=hydrochloride
HOBt=1-hydroxybenzotrizole
Hrs=hours
HPLC=high performance liquid chromatography
Hunig's base=N,N-Diisopropylethylamine
Lawesson's reagent=2,4-bis(4-methoxyphenyl)-1,3,2,4-dithiadiphosphetane-2,4-disulfide
MIC=minimum inhibitory concentration
mL=milliliter
MS=mass spectrometry
MRSA=methicillin-resistant Staphylococcus aureus
NMR=nuclear magnetic resonance
Ns=nitrobenzenesulfonyl
Pa=Pseudomonas aeruginosa
Prep=preparative
Ppm=parts per million
Py=pyridine
sat.=saturated
rt=room temperature
TBAF=tetrabutylammonium fluride
TBS=t-butyldimethylsilyl
TES=triethylsilyl
TEA=triethylamine
TEMPO=2,2,6,6-tetramethyl-1-piperidinyloxy, free radical
THF=tetrahydrofuran
TFA=trifluoroacetic acid
TMS=trimethylsilyl
TLC=thin layer chromatography
VAP=Ventilator-Associated Pneumonia

The compounds of Formula (I) can be prepared from intermediate 1, according to the following reaction schemes and examples, or modifications thereof, using readily available starting materials, reagents and conventional synthetic procedures including, for example, procedures described in U.S. Pat. No. 7112592 and WO2009/091856. Compound 3 can be synthesized following standard isoxazole ring formation chemistry from the chlorooxime intermediate 2d, which can be prepared from ester intermediate 1 (see, e.g., Abele, E.; Lukevics, E. Heterocycles 2000, 53, 2285-2336; Barr, L.; Lincoln, S. F.; Easton, C. J. Chemistry-A European Journal 2006, 12, 8571-8580; Walker, D. G.; Brodfuehrer, P. R.; Brundidge, S. P. Shih, K. M.; Sapino, C. Jr. J. Org. Chem. 1988, 53, 983-991 and references cited therein).

It may be necessary to protect certain functionalities in the molecule depending on the nature of the R¹ group. Protecting these functionalities should be within the expertise of one skilled in the art. See, e.g. P. G. M. Wuts and T. W. Greene, Protective Groups in Organic Synthesis, Fourth Edition, John Wiley and Sons, 2006, hereafter Greene.

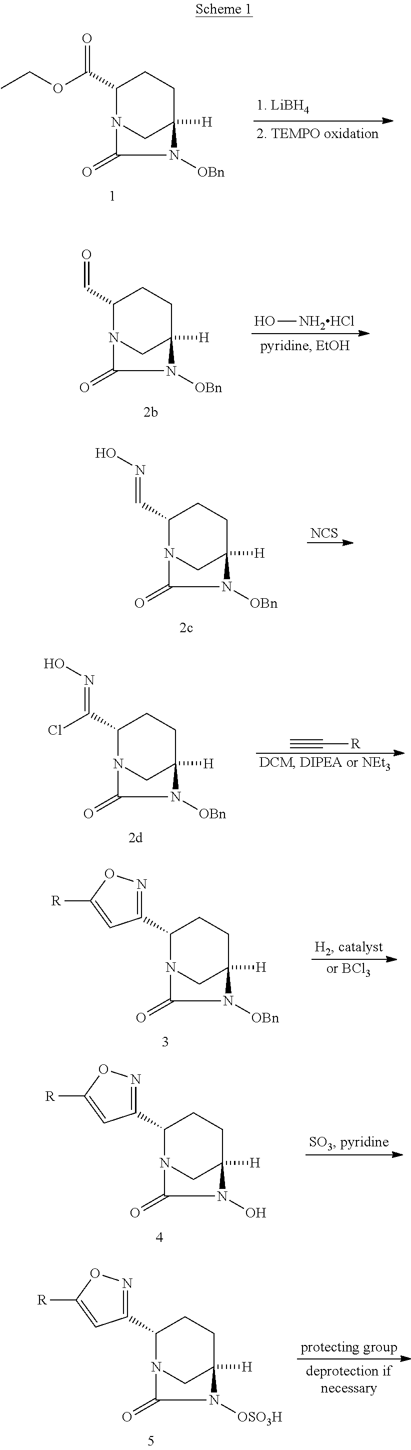

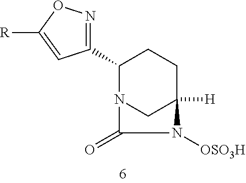

The benzylic ether protecting group in 3 can be removed via standard hydrogenolysis conditions, such as, but not limited to, Pd/H₂ in MeOH or THF or by acid-catalysed hydrolysis, such as, but not limited to, BCl₃ in DCM to provide the hydroxy-urea intermediate 4, which can be used directly in the next step without further purification. Sulfation of 4 can be achieved by treatment with a sulfating reagent, such a, but not limited to, SO₃.pyridine complex, in an appropriate solvent, such as pyridine, DMF or DMAc at a temperature of 0-80° C., preferable at room temperature. Compound 5 can then be isolated and purified via conventional methods. For example, 5 can be purified by standard reverse phase prep-HPLC using an appropriate buffer system, i.e. ammonium formate buffer. In some cases, 5 can be purified by normal phase silica gel chromatography after converting to an appropriate salt form, such as sulfate tetrabutyl ammonium salt. The tetrabutyl ammonium salt can then be converted to a sodium salt by cation exchange. When a protecting group(s) is present in the sidechain (i.e. Boc or Fmoc for amine and guanidine protection, TBS or TES for alcohol protection, etc), a deprotection step is needed to convert 5 to its final product 6, which can be purified by reverse phase prep-HPLC using the conditions mentioned above. For example, for N-Boc deprotection, 5 can be treated with an acid, such as TFA, in an appropriate solvent, such as DCM at a temperature of 0-30° C., preferable at 0° C. to rt to give 6. For an O-TBS, or O-TES deprotection, a fluoride reagent such as HF.pyridine, HF.NEt₃, or TBAF can be used. For Fmoc deprotection, amines, such as diethylamine, DBU, piperidine, etc can be used.

### EXAMPLES

The specific examples which follow illustrate the synthesis of certain compounds. The methods disclosed may be adopted to variations in order to produce compounds of Formula (I), but not otherwise specifically disclosed. Further, the disclosure includes variations of the methods described herein to produce the compounds of Formula (I) that would be understood by one skilled in the art based on the instant disclosure.

All temperatures are understood to be in Centigrade (C) when not specified. The nuclear magnetic resonance (NMR) spectral characteristics refer to chemical shifts (γ) expressed in parts per million (ppm) versus tetramethylsilane (TMS) as reference standard. The relative area reported for the various shifts in the proton NMR spectral data corresponds to the number of hydrogen atoms of a particular functional type in the molecule. The nature of the shifts as to multiplicity is reported as broad singlet (br s), broad doublet (br d), singlet (s), multiplet (m), doublet (d), quartet (q), doublet of doublet (dd), doublet of triplet (dt), and doublet of quartet (dq). The solvents employed for taking NMR spectra are DMSO-d6 (perdeuterodimethysulfoxide), D₂O (deuterated water), CDCl₃ (deuterochloroform) and other conventional deuterated solvents. The prep-HPLC conditions are: Waters SunFire® C18 (30×100 mm, 5 μm OBD) column; flow rate: 30-80 mL/minute, ELSD or Mass-triggered fraction collection; sample loading: Each injection loading varied from 30-300 mg for different crude samples depending on their solubility and purity profiles; Solvent system using ammonium formate buffer: solvent A: water with 20 mM ammonium formate, solvent B: 85% of acetonitrile in water with 20 mM ammonium formate. Solvent system using NH₄HCO₃ buffer: solvent A: water with 10 mM NH₄HCO₃, solvent B: acetonitrile. Solvent system using NH₄OH buffer: solvent A: water with 0.1% NH₄OH, solvent B: acetonitrile with 0.1% NH₄OH.

### Example 1

### Synthesis of (2S,5R)-ethyl 6-(benzyloxy)-7-oxo-1,6-diazabicyclo[3.2.1]octane-2-carboxylate (Intermediate Compound 1)

c

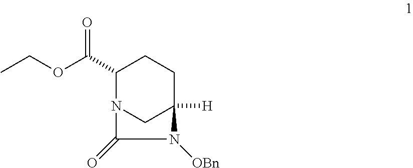 [SMILES: CCOC(=O)[C@@H]1CC[C@@H]2CN1C(=O)N2OCc1ccccc1]

Step 1: Synthesis of (S)-1-tert-butyl 2-ethyl 5-oxopiperidine-1,2-dicarboxylate

Method A:

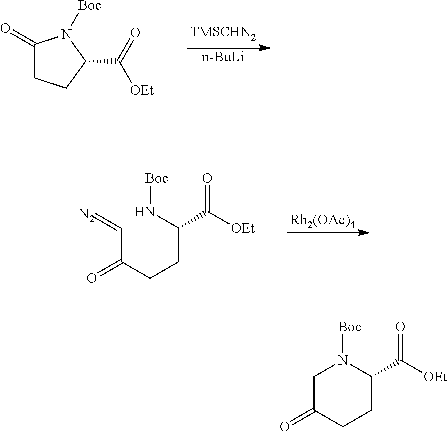

n-BuLi (600 mL, 1.5 mol) was added dropwise to a solution of TMSCHN₂ (690 mL, 1.38 mol) in dry THF (3 L) at −78° C., and the mixture was stirred at −78° C. for 30 minutes. The mixture was then transferred to a solution of (S)-1-tert-butyl 2-ethyl 5-oxopyrrolidine-1,2-dicarboxylate (300 g, 1.17 mol) in dry THF (3 L) via cannula, and the mixture was stirred at −78° C. for 30 minutes. The reaction mixture was then quenched with sat. NH₄Cl solution, and extracted with DCM (3×). The combined organic layer was concentrated under reduced pressure and the crude product was purified by silica gel column chromatography (3:1 petroleum ether/EtOAc) to afford (S)-ethyl 2-((tert-butoxycarbonyl)amino)-6-diazo-5-oxohexanoate (262 g, 75%) as a yellow solid.

A solution of (S)-ethyl 2-((tert-butoxycarbonyl)amino)-6-diazo-5-oxohexanoate (350 g, 1.18 mol) in DCM (1500 mL) was added to a 0° C. solution of Rh₂(OAc)₄ (3.5 g, 7.9 mmol) in DCM (750 mL). The reaction mixture was then stirred at 20° C. overnight and then concentrated under reduced pressure. The crude sample was purified by silica gel column chromatography (5:1 petroleum ether/EtOAc) to afford (S)-1-tert-butyl 2-ethyl 5-oxopiperidine-1,2-dicarboxylate (175.9 g, 55%) as a yellow oil.

Method B:

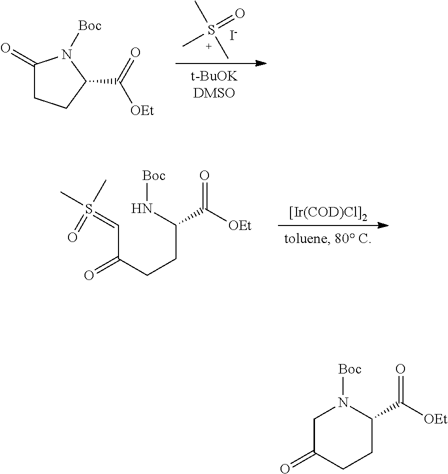

t-BuOK (330 g, 2.9 mol) was added to a solution of trimethylsulfoxonium iodide (750 g, 3.5 mol) in dry DMSO (3 L) and the mixture was stirred at rt for 1 h. (S)-1-tert-Butyl 2-ethyl 5-oxopyrrolidine-1,2-dicarboxylate (900 g, 3.5 mol) was added and the mixture was stirred at rt for 2-3 hrs. Water was added to quench the reaction and the mixture was extracted with EtOAc (5×). The combined organic layer was concentrated under reduced pressure and the crude sample was purified by silica gel column chromatography (1:1 petroleum ether/EtOAc then 1:10 MeOH/DCM) to afford sulfoxonium ylide intermediate (977 g, 80%) as a white solid.

A solution of sulfoxonium ylide intermediate (156 g, 0.446 mol) and [Ir(COD)Cl]₂ (3 g, 4.46 mmol) in toluene (4 L) was degassed by bubbling nitrogen through the solution for 10 minutes. The reaction mixture was heated to 80-90° C. for 2-3 hrs and then cooled to 20° C. The toluene was concentrated under reduced pressure and the resulting residue was purified by silica gel column chromatography (10:1 to 3:1 gradient elution petroleum ether/EtOAc) to afford (S)-1-tert-butyl 2-ethyl 5-oxopiperidine-1,2-dicarboxylate (140 g, 57.8%) as a yellow oil.

Step 2: Synthesis of (2S,5S)-1-tert-butyl 2-ethyl 5-hydroxypiperidine-1,2-dicarboxylate

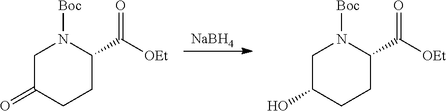 [SMILES: CCOC(=O)[C@@H]1CCC(=O)CN1C(=O)OC(C)(C)C ; "NaBH4" ; CCOC(=O)[C@@H]1CC[C@H](O)CN1C(=O)OC(C)(C)C]

NaBH₄ (36 g, 1.0 mol) was added in portions to a −40° C. solution of (S)-1-tert-butyl 2-ethyl 5-oxopiperidine-1,2-dicarboxylate (250 g, 0.92 mol) in EtOH (1500 mL). The reaction mixture was then stirred at −40° C. for 0.5 hr then quenched with 10% HOAc solution. After diluting with water, the mixture was extracted with DCM (3×). The combined organic layer was concentrated under reduced pressure and purified by silica gel column chromatography (1:1 petroleum ether/EtOAc) to afford (2S,5S)-1-tert-butyl 2-ethyl 5-hydroxypiperidine-1,2-dicarboxylate (205 g, 80%) as a yellow oil.

Step 3: Synthesis of (2S,5R)-1-tert-butyl 2-ethyl 5-(N-(benzyloxy)-2-nitrophenylsulfonamido)piperidine-1,2-dicarboxylate

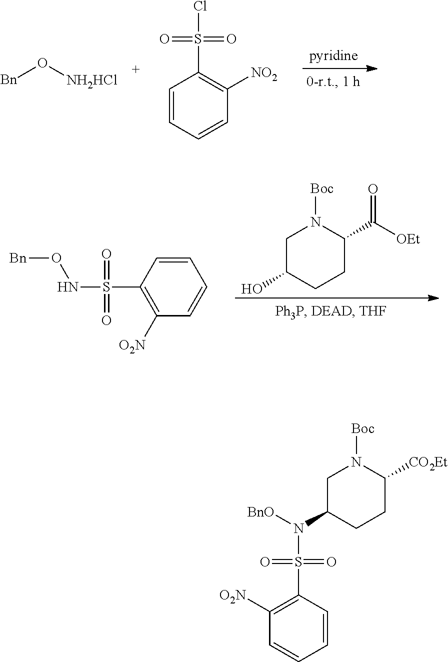

A solution of 2-nitrobenzene-1-sulfonyl chloride (500 g, 2.26 mol) in pyridine (1500 mL) was added dropwise to a 0° C. solution of O-benzylhydroxylamine hydrochloride (400 g, 2.51 mol) in pyridine (1500 mL). The reaction mixture was allowed to warm to room temperature then was stirred at 20° C. overnight. The mixture was concentrated under reduced pressure, diluted with DCM and washed with HCl (10%, 3×). The combined organic layer was concentrated under reduced pressure and re-crystallized with DCM to afford N-(benzyloxy)-2-nitrobenzenesulfonamide (485 g, 62.6%) as a yellow solid.

To a solution of N-(benzyloxy)-2-nitrobenzenesulfonamide (212 g, 0.69 mol) in THF (1000 mL) was added (2S,5S)-1-tert-butyl 2-ethyl 5-hydroxypiperidine-1,2-dicarboxylate (171 g, 0.63 mol) and PPh₃ (275 g, 1.05 mol), followed by dropwise addition of a solution of DEAD (195 g, 1.12 mol) in THF (500 mL). The mixture was then stirred at 20° C. overnight. The reaction mixture was then concentrated under reduced pressure and purified by silica gel column chromatography (3:1 petroleum ether/EtOAc) to afford (2S,5R)-1-tert-butyl 2-ethyl 5-(N-(benzyloxy)-2-nitrophenylsulfonamido)piperidine-1,2-dicarboxylate (283.8 g, 80%) as a yellow oil.

Step 4: Synthesis of (2S,5R)-1-tert-butyl 2-ethyl 5-((benzyloxy)amino)piperidine-1,2-dicarboxylate

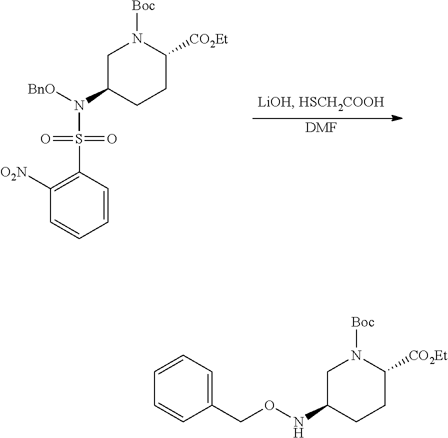

LiOH.H₂O (95 g, 2.3 mol) and 2-mercaptoacetic acid (124 g, 1.3 mol) were added to a solution of (2S,5R)-1-tert-butyl 2-ethyl 5-(N-(benzyloxy)-2-nitrophenylsulfonamido)piperidine-1,2-dicarboxylate (251 g, 0.45 mol) in DMF (1200 mL). The reaction mixture was then stirred at 20° C. overnight. The reaction mixture was diluted with water and extracted with EtOAc (3×). The combined organic layer was washed with saturated sodium chloride (3×), concentrated under reduced pressure and purified by silica gel column chromatography (3:1 petroleum ether/EtOAc) to afford (2S,5R)-1-tert-butyl 2-ethyl 5-((benzyloxy)amino)piperidine-1,2-dicarboxylate (122.9 g, 85%) as a yellow solid.

Step 5: Synthesis of (2S,5R)-ethyl 5-((benzyloxy)amino)piperidine-2-carboxylate

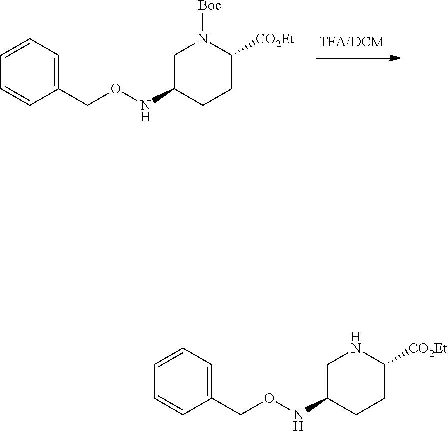 [SMILES: CCOC(=O)[C@@H]1CC[C@@H](NOCc2ccccc2)CN1C(=O)OC(C)(C)C ; CCOC(=O)[C@@H]1CC[C@@H](NOCc2ccccc2)CN1]

TFA (600 mL) was added to a solution of (2S,5R)-1-tert-butyl 2-ethyl 5-((benzyloxy)amino)piperidine-1,2-dicarboxylate (263 g, 0.7 mol) in DCM (600 mL) at 20° C. The mixture was stirred at rt overnight and then concentrated under reduced pressure. The crude product was adjusted to pH 10 with sat. NaHCO₃ solution then extracted with DCM (3×). The combined organic layer was concentrated under reduced pressure and purified by silica gel column chromatography (20:1 DCM/MeOH) to afford (2S,5R)-ethyl 5-((benzyloxy)amino)piperidine-2-carboxylate (184.9 g, 95%) as a yellow oil.

Step 6: Synthesis of (2S,5R)-ethyl 6-(benzyloxy)-7-oxo-1,6-diazabicyclo[3.2.1]octane-2-carboxylate

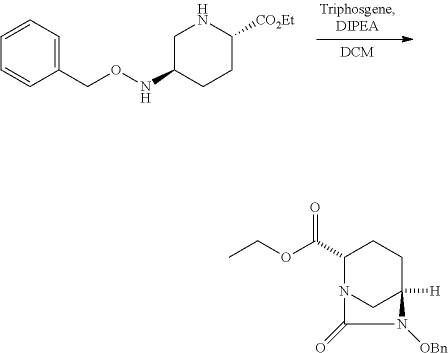 [SMILES: CCOC(=O)[C@@H]1CC[C@@H](NOCc2ccccc2)CN1 ; CCOC(=O)[C@@H]1CC[C@@H]2CN1C(=O)N2OCc1ccccc1]

Triphosgene (21.3 g, 72 mmol) was added in portions to a 0° C. solution of (2S,5R)-ethyl 5-((benzyloxy)amino)piperidine-2-carboxylate (50 g, 0.18 mol) and DIPEA (128 mL, 0.72 mol) in DCM (2000 mL). The reaction mixture was allowed to warm to rt. After stirring at rt overnight, the reaction mixture was washed with H₃PO₄ (10%), sat. NaHCO₃ and saturated NaCl. The combined organic layer was concentrated under reduced pressure and purified by silica gel column chromatography (3:1 petroleum ether/EtOAc) to afford (2S,5R)-ethyl 6-(benzyloxy)-7-oxo-1,6-diazabicyclo[3.2.1]octane-2-carboxylate (27.4 g, 50%) as a yellow solid. ¹H NMR (400 Mz, CDCl₃): δ 7.43-7.36 (m, 5H), 5.06 (d, J=11.4 Hz, 1H), 4.90 (d, J=11.4 Hz, 1H), 4.24 (q, J=7.1 Hz, 2H), 4.11-4.08 (m, 1H), 3.32-3.31 (m, 1H), 3.08-3.05 (m, 1H), 2.93 (d, J=11.9 Hz, 1H), 2.14-2.05 (m, 2H), 2.05-2.00 (m, 1H), 1.71-1.63 (m, 1H), 1.29 (t, J=7.1 Hz, 3H).

### Example 2

### Synthesis of (2S,5R)-6-(benzyloxy)-7-oxo-1,6-diazabicyclo[3.2.1]octane-2-carbaldehyde (Intermediate Compound 2b)

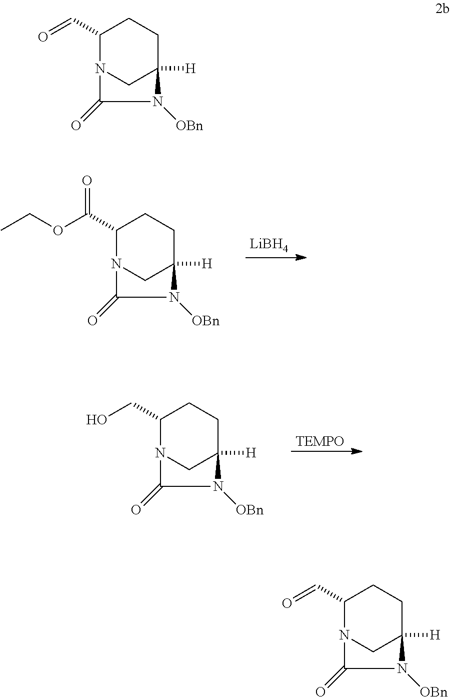 [SMILES: O=C[C@@H]1CC[C@@H]2CN1C(=O)N2OCc1ccccc1 ; CCOC(=O)[C@@H]1CC[C@@H]2CN1C(=O)N2OCc1ccccc1 ; "LiBH4" ; O=C1N2C[C@@H](CC[C@H]2CO)N1OCc1ccccc1 ; "TEMPO" ; O=C[C@@H]1CC[C@@H]2CN1C(=O)N2OCc1ccccc1]

LiBH₄ (0.54 g, 24.67 mmol) was added to a −10° C. solution of (2S,5R)-ethyl 6-(benzyloxy)-7-oxo-1,6-diazabicyclo[3.2.1]octane-2-carboxylate (5 g, 16.44 mmol) in MeOH (50 mL). After 15 minutes another portion of LiBH₄ (0.54 g, 24.67 mmol) was added and the mixture was stirred at −10 to 0° C. for 4-5 h. The reaction mixture was carefully quenched by addition of sat. NaH₂PO₄ (50 mL) at 0° C. The mixture was diluted with water (20 mL) and extracted with DCM (3×). The combined organic layer was concentrated and purified by silica gel column chromatography (gradient elution 0-100% EtOAc/petroleum ether, then 0-2% MeOH/EtOAc) to give (2S,5R)-6-(benzyloxy)-2-(hydroxymethyl)-1,6-diazabicyclo[3.2.1]octan-7-one (3.8 g, 88%) as a white solid. ESI-MS (EI⁺, m/z): 263.1. ¹H NMR (500M, CDCl₃): 7.44-7.35 (m, 5H), 5.05 (d, J=11.5 Hz, 1H), 4.90 (d, J=11.5 Hz, 1H), 3.73-3.69 (m, 1H), 3.61-3.58 (m, 2H), 3.33 (m, 1H), 3.01 (br d, J=12.0 Hz, 1H), 2.91 (m, 1H), 2.03-1.95 (m, 2H), 1.58-1.54 (m, 1H), 1.39-1.24 (m, 1H).

TEMPO (48 mg, 0.3 mmol) was added in portions to a 0° C. solution of (2S,5R)-6-(benzyloxy)-2-(hydroxymethyl)-1,6-diazabicyclo[3.2.1]octan-7-one (7.8 g, 30 mmol) and 1,3,5-trichloro-1,3,5-triazinane-2,4,6-trione (7.0 g, 30 mmol) in DCM (100 mL). The mixture was stirred at 0° C. for 2 h, and filtered through Celite®. The filtrate was dried over Na₂SO₄ and concentrated to afford (2S,5R)-6-(benzyloxy)-7-oxo-1,6-diazabicyclo[3.2.1]octane-2-carbaldehyde (7.0 g, 90%) as a yellow oil. ESI-MS (EI⁺, m/z): 261.1. ¹H NMR (500M, CDCl₃): 9.74 (s, 1H), 7.45-7.36 (m, 5H), 5.07 (d, J=11.5 Hz, 1H), 4.92 (d, J=11.5 Hz, 1H), 3.89 (d, J=8.0 Hz, 1H), 3.27 (m, 1H), 3.21-3.05 (m, 1H), 2.56 (d, J=12.0 Hz, 1H), 2.20-2.15 (m, 1H), 2.05-2.01 (m, 1H), 1.95-1.93 (m, 1H), 1.49-1.46 (m, 1H).

### Example 3

### Synthesis of (E)-6-(benzyloxy)-7-oxo-1,6-diazabicyclo[3.2.1]octane-2-carbaldehyde oxime (Intermediate Compound 2c)

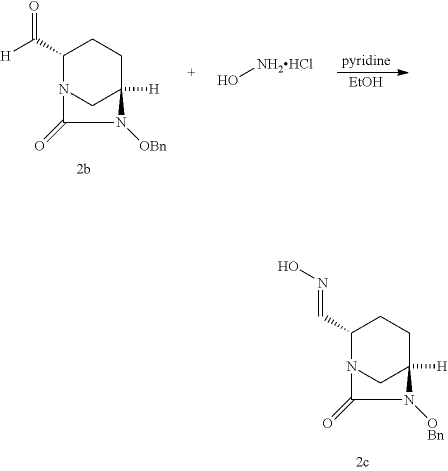

A solution of (2S,5R)-6-(benzyloxy)-7-oxo-1,6-diazabicyclo[3.2.1]octane-2-carbaldehyde (510 mg, 1.96 mmol), hydroxylamine hydrochloride (158 mg, 2.27 mmol) and pyridine (621 mg, 7.85 mmol) in EtOH (15 mL) was stirred at rt for 2 hrs. Then, the reaction mixture was concentrated and the residue was diluted with DCM (25 mL), washed with water (3×), and saturated sodium chloride, dried over Na₂SO₄ and concentrated. The residue was purified by silica gel column chromatography (3:1 to 3:2 petroleum ether/EtOAC) to afford (E)-6-(benzyloxy)-7-oxo-1,6-diazabicyclo[3.2.1]octane-2-carbaldehyde oxime (228 mg, 42%) as a white solid. ESI-MS (EI⁺, m/z): 276 [M+H]⁺.

### Example 4

### Synthesis of (2S,5R)-2-(5-(aminomethyl)isoxazol-3-yl)-7-oxo-1,6-diazabicyclo[3.2.1]octan-6-yl hydrogen sulfate (Compound 601)

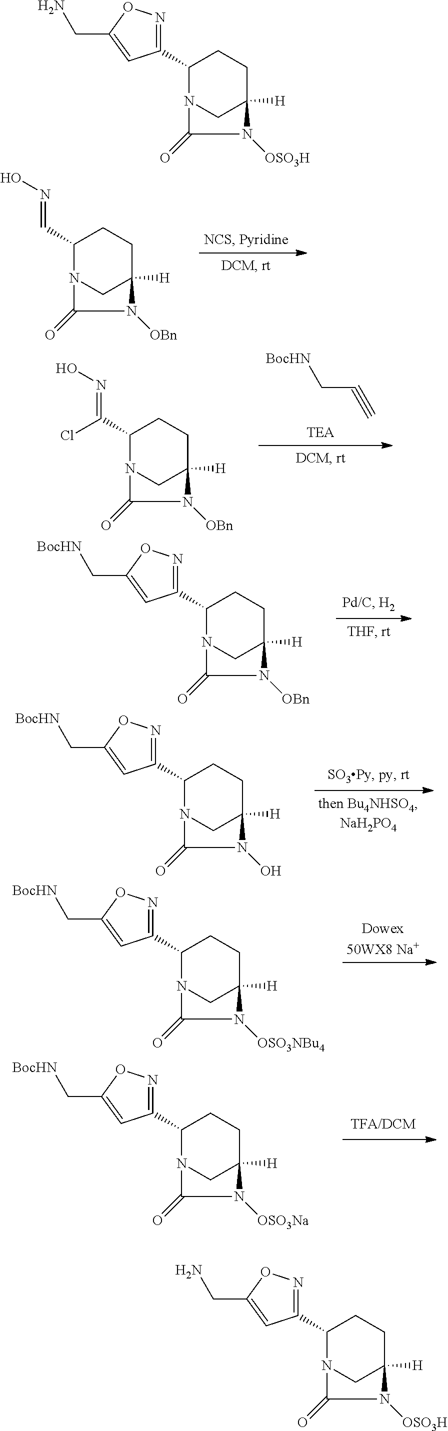

Step 1: NCS (295 mg, 2.2 mmol) was added to a solution of 6-(benzyloxy)-7-oxo-1,6-diaza-bicyclo[3.2.1]octane-2-carbaldehyde oxime (560 mg, 2.0 mmol) in dry DCM (15 mL) at rt. Pyridine (one drop) was then added, and the reaction mixture was stirred at rt for 18 hrs. The solution was evaporated to afford (2S,5R)-6-(benzyloxy)-N-hydroxy-7-oxo-1,6-diaza-bicyclo[3.2.1]octane-2-carbimidoyl chloride, which was directly used in the next step. ESI-MS (EI⁺, m/z): 274 [M-Cl]⁺.

Step 2: tert-Butyl prop-2-yn-1-ylcarbamate (0.37 g, 2.4 mmol) was added to the solution of (2S,5R)-6-(benzyloxy)-N-hydroxy-7-oxo-1,6-diaza-bicyclo[3.2.1]octane-2-carbimidoyl chloride (˜2.0 mmol) in dry DCM (20 mL), followed by the addition of TEA (0.31 mL, 2.2 mmol) in dry DCM (2.0 mL) over a period of 30 minutes. The reaction mixture was stirred at rt overnight, then the mixture was diluted with EtOAc, washed with water and saturated sodium chloride. The organic layer was dried over Na₂SO₄, concentrated and purified by silica gel column chromatography (1:2 petroleum ether/EtOAc) to afford tert-butyl ((3-((2S,5R)-6-(benzyloxy)-7-oxo-1,6-diazabicyclo[3.2.1]octan-2-yl)isoxazol-5-yl)methyl)carbamate (180 mg, 21% for two steps) as a white solid. ESI-MS (EI⁺, m/z): 429.1 [M+H]⁺. ¹H NMR (500 MHz, CDCl₃): δ 7.45-7.35 (m, 5H), 6.24 (s, 1H), 5.09 (d, J=11.5 Hz, 1H), 4.93 (d, J=11.5 Hz, 1H), 4.58 (d, J=7.5 Hz, 1H), 4.43 (m, 2H), 3.32 (s, 1H), 2.86-2.84 (m, 1H), 2.68 (d, J=11.5 Hz, 1H), 2.34-2.30 (m, 1H), 2.23-2.18 (m, 1H), 2.10-2.04 (m, 1H), 1.82-1.78 (m, 1H), 1.45 (s, 9H).

Step 3: To a solution of tert-butyl (3-((2S,5R)-6-(benzyloxy)-7-oxo-1,6-diaza-bicyclo[3.2.1]octan-2-yl)isoxazol-5-yl)methylcarbamate (210 mg, 0.5 mmol) in THF (5 mL) was added 10% Pd/C (100 mg). The reaction mixture was then filtered and concentrated to afford tert-butyl ((3-((2S,5R)-6-hydroxy-7-oxo-1,6-diazabicyclo[3.2.1]octan-2-yl)isoxazol-5-yl)methyl)carbamate (180 mg, 99%) as a light yellow solid, which was used directly in the next step. ESI-MS (EI⁺, m/z): 339.1 [M+H]⁺.

Step 4: To a solution of tert-butyl (3-((2S,5R)-6-hydroxy-7-oxo-1,6-diaza-bicyclo[3.2.1]octan-2-yl)isoxazol-5-yl)methylcarbamate (180 mg, 0.5 mmol) in dry pyridine (2.5 mL) was added SO₃.Py (480 mg, 3.0 mmol). The mixture was stirred at rt for 3 hrs and then concentrated under reduced pressure. The residue was re-dissolved in aqueous NaH₂PO₄ (1.5 M, 20 mL) then tetrabutylammonium hydrogensulphate (230 mg, 0.67 mmol) was added. The mixture was stirred at rt for 20 minutes then extracted with EtOAc (4×). The combined organic layer was dried and concentrated and the residue was purified by silica gel column chromatography (gradient elution 10:1 to 3:1 DCM/acetone) to afford tetrabutylammonium (2S,5R)-2-(5-(((tert-butoxycarbonyl)amino)methyl)isoxazol-3-yl)-7-oxo-1,6-diazabicyclo[3.2.1]octan-6-yl sulfate (180 mg, 54%) as a white solid. ESI-MS (EI⁻, m/z): 417.0 [M−H]⁻. ¹H NMR (500 MHz, CDCl₃): δ 6.28 (s, 1H), 4.97 (bs, 1H), 4.54 (d, J=7 Hz, 1H), 4.44-4.30 (m, 2H), 4.35 (m, 1H), 3.35-3.28 (m, 8H), 3.19-3.17 (m, 1H), 2.76 (d, J=11.5 Hz, 1H), 2.36-2.32 (m, 1H), 2.27-2.24 (m, 1H), 2.18-2.12 (m, 1H), 1.87-1.81 (m, 1H), 1.71-1.65 (m, 8H), 1.50-1.47 (m, 17H), 1.01 (t, J=7.0 Hz, 12H).

Step 5, Resin Exchange: Tetrabutylammonium (2S, 5R)-2-(5-(tert-butoxycarbonylaminomethyl)-isoxazol-3-yl)-7-oxo-1,6-diaza-bicyclo[3.2.1]octan-6-yl sulfate (180 mg, 0.27 mmol) was dissolved in a minimum amount of HPLC grade water (˜10 mL) and passed through a column of 20 g of DOWEX 50WX 8 Na⁺ resin (the resin was pre-washed with >0.5 L of HPLC grade water) and eluted with HPLC grade water to afford sodium (2S,5R)-2-(5-(((tert-butoxycarbonyl)amino)methyl)isoxazol-3-yl)-7-oxo-1,6-diazabicyclo[3.2.1]octan-6-yl sulfate (109 mg, 92%) was obtained after lyophilization as a white solid. ESI-MS (E⁻, m/z): 417.0 [M−H]⁻. ¹H NMR (500 MHz, D₂O): δ 6.32 (s, 1H), 4.61 (d, J=6.5 Hz, 1H), 4.32 (s, 2H), 4.13 (m, 1H), 3.10-3.08 (m, 1H), 2.91 (d, J=12.5 Hz, 1H), 2.21-2.17 (m, 1H), 2.09-2.02 (m, 2H), 1.86-1.82 (m, 1H), 1.34 (s, 9H).

Step 6: TFA (0.40 mL) was added to a 0° C. solution of sodium (2S, 5R)-2-(5-(tert-butoxycarbonylaminomethyl)-isoxazol-3-yl)-7-oxo-1,6-diaza-bicyclo[3.2.1]octan-6-yl sulfate (54 mg, 0.12 mmol) in dry DCM (1.2 mL). The reaction mixture was stirred at 0° C. for 30 minutes to 1 h and then diluted with diethyl ether. The precipitate was collected via centrifugation, washed with ether (3×) and further dried under high vacuum to provide (2S,5R)-2-(5-(aminomethyl)isoxazol-3-yl)-7-oxo-1,6-diazabicyclo[3.2.1]octan-6-yl hydrogen sulfate as a TFA salt (˜30 mg). ESI-MS (EI⁺, m/z): 319.2. The TFA salt (˜30 mg) was further purified by prep-HPLC using ammonium formate buffer to provide (2S,5R)-2-(5-(aminomethyl)isoxazol-3-yl)-7-oxo-1,6-diazabicyclo[3.2.1]octan-6-yl hydrogen sulfate (601, 10 mg, 26%) as a white solid. ESI-MS (EI⁺, m/z): 319.21. ¹H NMR (300 MHz, D₂O) δ 6.61 (s, 1H),4.68-4.65 (m, 1H), 4.35 (s, 2H), 4.17-4.14 (m, 1H), 3.14-3.10 (m, 1H), 2.95-2.91 (m, 1H), 2.31-1.80 (m, 4H).

### Example 5

### Synthesis of (2S,5R)-2-(5-(guanidinomethyl)isoxazol-3-yl)-7-oxo-1,6-diazabicyclo[3.2.1]octan-6-yl hydrogen sulfate (Compound 603)

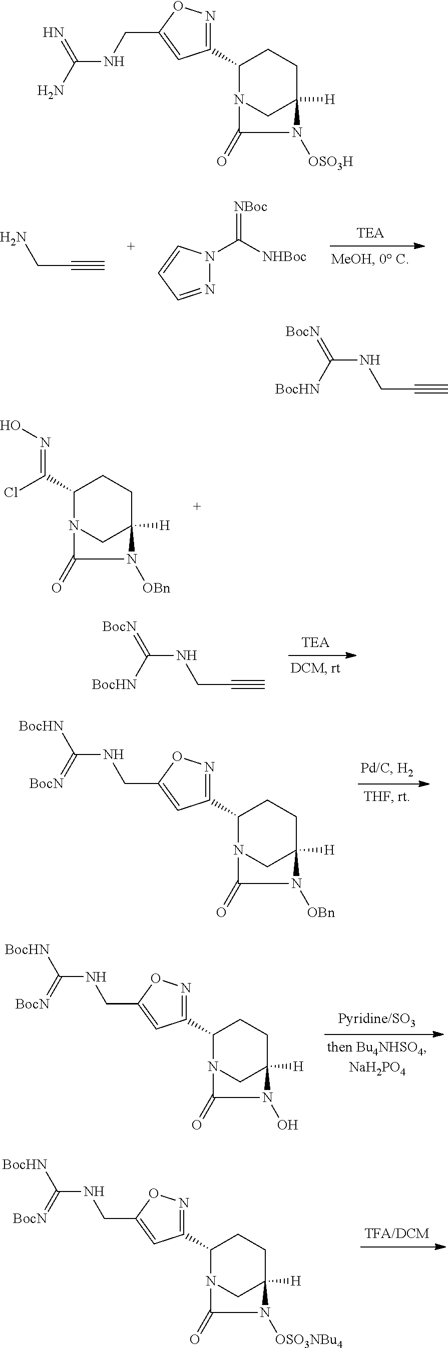

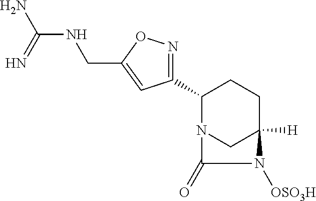

Step 1: Synthesis of 1-(2,3-bis(tert-butoxycarbonyl)guanidino)prop-2-yne: Di-tert-butyl (1H-pyrazol-1-yl)methanediylidenedicarbamate (1.71 g, 5.5 mmol) was added to a 0° C. solution of prop-2-yn-1-amine (275 mg, 5.0 mmol) and TEA (1.5 g, 15 mmol) in MeOH (40 mL). The reaction mixture was stirred at 0° C. for 3 hrs and then concentrated under reduced pressure. The residue was purified by silica gel column chromatography (1:10 EtOAc/Hexanes) to give 1-(2,3-bis(tert-butoxycarbonyl)guanidino)prop-2-yne (1.4 g, 93%) as a white solid. ESI-MS (EI⁺, m/z): 298.1 [M+H]⁺.

Step 2: Synthesis of (2S,5R)-6-(benzyloxy)-2-(5-((2,3-bis(tert-butoxycarbonyl)guanidino)methyl)isoxazol-3-yl)-1,6-diaza-bicyclo[3.2.1]octan-7-one: 1-(2,3-bis(tert-butoxycarbonyl)guanidino)prop-2-yne (362 mg, 1.22 mmol) was added to the solution of (2S,5R)-6-(benzyloxy)-N-hydroxy-7-oxo-1,6-diaza-bicyclo[3.2.1]octane-2-carbimidoyl chloride (473 mg, 1.53 mmol) in dry DCM (20 mL) at rt, followed by dropwise addition of TEA (0.16 g, 1.53 mmol). The mixture was stirred at rt for 2 hrs and then concentrated. The residue was purified by silica gel column chromatography (gradient elution 1:15 to 1:5 EtOAc/Hexanes) to give (2S,5R)-6-(benzyloxy)-2-(5-((2,3-bis(tert-butoxycarbonyl)guanidino)methyl)isoxazol-3-yl)-1,6-diaza-bicyclo[3.2.1]octan-7-one (190 mg, 22% for two steps) as a white solid. ESI-MS (EI⁺, m/z): 571.2 [M+H]⁺. ¹H NMR (500 MHz, CDCl₃): δ 11.49 (s, 1H), 8.86 (bs, 1H), 7.47-7.34 (m, 5H), 6.32 (s, 1H), 5.09 (d, J=11.5 Hz, 1H), 4.93 (d, J=12.0 Hz, 1H), 4.87-4.76 (m, 2H), 4.59 (d, J=7.0 Hz, 1H), 3.36 (s, 1H), 2.87-2.85 (m, 1H), 2.70 (d, J=12 Hz, 1H), 2.35-2.30 (m, 1H), 2.24-2.18 (m, 1H), 2.10-2.05 (m, 1H), 1.81-1.78 (m, 1H), 1.50 (s, 18H).

Step 3: Synthesis of (2S,5R)-6-(hydroxy)-2-(5-((2,3-bis(tert-butoxycarbonyl)guanidino)methyl)isoxazol-3-yl)-1,6-diaza-bicyclo[3.2.1]octan-7-one: To a solution of (2S,5R)-6-(benzyloxy)-2-(5-((2,3-bis(tert-butoxycarbonyl)guanidino)methyl)isoxazol-3-yl)-1,6-diaza-bicyclo[3.2.1]octan-7-one (240 mg, 0.42 mmol) in THF (20 mL) was added 10% Pd/C (120 mg). The mixture was stirred under H₂ atmosphere at rt for 1.5 hrs. The reaction mixture was then filtered and concentrated to afford (2S,5R)-6-(hydroxy)-2-(5-((2,3-bis(tert-butoxycarbonyl)guanidino)methyl)isoxazol-3-yl)-1,6-diaza-bicyclo[3.2.1]octan-7-one (200 mg, 99%) as white solid, which was directly used in the next step. ESI-MS (EI⁺, m/z): 481.2 [M+H]⁺.

Step 4: Synthesis of tetrabutylammonium(2S,5R)-2-(5-((2,3-bis(tert-butoxycarbonyl)guanidino)methyl)isoxazol-3-yl)-7-oxo-1,6-diaza-bicyclo[3.2.1]octan-6-yl sulfate:

To a solution of (2S,5R)-6-(hydroxy)-2-(5-((2,3-bis(tert-butoxycarbonyl)guanidino)methyl)isoxazol-3-yl)-1,6-diaza-bicyclo[3.2.1]octan-7-one (200 mg, 0.42 mmol) in dry pyridine (2.5 mL) was added SO₃.Py (400 mg, 2.5 mmol). The mixture was stirred at rt for 3 hrs then concentrated under reduced pressure. The residue was re-dissolved in aqueous NaH₂PO₄ (1.5 M, 20 mL). Tetrabutylammonium hydrogensulphate (200 mg, 0.58 mmol) was added. The mixture was stirred at rt for 20 minutes then was extracted with EtOAc (4×). The combined organic layer was dried and concentrated and the residue was purified by silica gel column chromatography (gradient elution 10:1 to 5:1 DCM/acetone) to give tetrabutylammonium (2S,5R)-2-(5-((2,3-bis(tert-butoxycarbonyl)guanidino)methyl)isoxazol-3-yl)-7-oxo-1,6-diaza-bicyclo[3.2.1]octan-6-yl sulfate (220 mg, 66%) as a white solid. ESI-MS (EI⁻, m/z): 559.0 [M−H]⁻. ¹H NMR (500 MHz, CDCl₃): δ 11.48 (s, 1H), 8.78 (bs, 1H), 6.34 (s, 1H), 4.77-4.76 (m, 2H), 4.55 (d, J=7.5 Hz, 1H), 4.35 (m, 1H), 3.38-3.28 (m, 8H), 3.20-3.18 (m, 1H), 2.78 (d, J=11.5 Hz, 1H), 2.37-2.30 (m, 1H), 2.28-2.24 (m, 1H), 2.17-2.11 (m, 1H), 1.88-1.82(m, 1H), 1.71-1.63 (m, 8H), 1.50-1.44 (m, 26H), 1.01 (t, J=7.0 Hz, 12H).

Step 5: TFA (2.20 mL) was added to a 0° C. solution of tetrabutylammonium (2S,5R)-2-(5-((2,3-bis(tert-butoxycarbonyl)guanidino)methyl)isoxazol-3-yl)-7-oxo-1,6-diaza-bicyclo[3.2.1]octan-6-yl sulfate (465 mg, 0.60 mmol) in dry DCM (6.60 mL). The reaction mixture was allowed to warm to rt. The reaction mixture was stirred at rt for 2 hrs and then diluted with ether. The precipitate was collected via centrifugation, washed with diethyl ether (3×) and further dried under high vacuum. The crude TFA salt was purified by prep-HPLC using ammonium formate buffer to provide (2S,5R)-2-(5-(guanidinomethyl)isoxazol-3-yl)-7-oxo-1,6-diazabicyclo[3.2.1]octan-6-yl hydrogen sulfate (603, 60 mg, 20%) as a white solid. ESI-MS (EI⁺, m/z): 361.2. ¹H NMR (300 MHz, D₂O/DMSO-d6) δ 6.58 (s, 1H), 4.74-4.71 (m, 1H), 4.67 (s, 2H), 4.23 (br s, 1H), 3.22-3.17 (m, 1H), 2.99 (d, J=12.0 Hz, 1H), 2.36-2.26 (m, 1H), 2.24-2.10 (m, 2H), 2.02-1.91 (m, 1H).

### Example 6

### Synthesis of (2S,5R)-2-(5-(2-aminoethyl)isoxazol-3-yl)-7-oxo-1,6-diazabicyclo[3.2.1]octan-6-yl hydrogen sulfate (Compound 602)

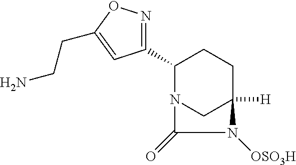

Following Steps 1-6 in Example 4, replacing tert-butyl prop-2-yn-1-ylcarbamate in Step 2 with tert-butyl but-3-yn-1-ylcarbamate; (2S,5R)-2-(5-(2-aminoethyl)-1,3,4-oxadiazol-2-yl)-7-oxo-1,6-diazabicyclo[3.2.1]octan-6-yl hydrogen sulfate TFA salt (602, 8 mg) was obtained as a light yellow solid. ESI-MS (EI⁺, m/z): 333.2. ¹H NMR (300 MHz, D₂O) δ 6.39 (s, 1H), 4.62 (d, J=6.8 Hz, 1H), 4.16-4.12 (m, 1H), 3.32 (t, J=7.1 Hz, 3H), 3.25-3.04 (m, 4H), 2.97-2.93 (m, 1H), 2.30-1.79 (m, 4H).

### Example 7

### Synthesis of (2S,5R)-2-(5-(2-guanidinoethyl)isoxazol-3-yl)-7-oxo-1,6-diazabicyclo[3.2.1]octan-6-yl hydrogen sulfate (Compound 604)

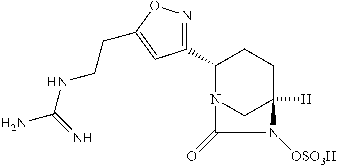

Following Steps 1-5 in Example 5, replacing prop-2-yn-1-amine in Step 1 with but-3-yn-1-amine; (2S,5R)-2-(5-(2-guanidinoethyl)isoxazol-3-yl)-7-oxo-1,6-diazabicyclo[3.2.1]octan-6-yl hydrogen sulfate (604, 48 mg) was obtained as a light yellow solid after prep-HPLC purification using ammonium formate buffer. ESI-MS (EI⁺, m/z): 375.1. ¹H NMR (300 MHz, D₂O/DMSO-d6) δ 6.33 (s, 1H), 4.62 (d, J=6.6 Hz, 1H), 4.14 (br s, 1H), 3.51 (t, J=6.4 Hz, 2H), 3.15-3.07 (m, 1H), 3.05 (t, J=6.4 Hz, 2H), 2.88 (d, J=6.4 Hz, 1H), 2.26-1.99 (m, 3H), 1.85-1.80 (m, 1H).

### Example 8

### Synthesis of (2S,5R)-7-oxo-2-(5-(piperidin-4-yl)isoxazol-3-yl)-1,6-diazabicyclo[3.2.1]octan-6-yl hydrogen sulfate (Compound 606)

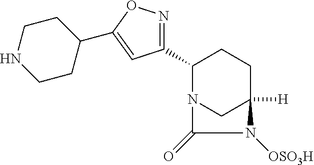

Following Steps 1-6 in Example 4, replacing tert-butyl prop-2-yn-1-ylcarbamate in Step 2 with tert-butyl 4-ethynylpiperidine-1-carboxylate, (2S,5R)-7-oxo-2-(5-(piperidin-4-yl)isoxazol-3-yl)-1,6-diazabicyclo[3.2.1]octan-6-yl hydrogen sulfate (606, 35 mg) was obtained as a white solid after prep-HPLC purification using ammonium formate buffer. ESI-MS (EI⁺, m/z): 371.3.

### Example 9

### Synthesis of (2S,5R)-2-(5-(1-carbamimidoylpiperidin-4-yl)isoxazol-3-yl)-7-oxo-1,6-diazabicyclo[3.2.1]octan-6-yl hydrogen sulfate (Compound 607)

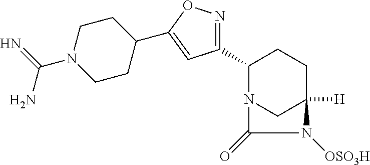

Following Steps 1-5 in Example 5, replacing prop-2-yn-1-amine in Step 1 with 4-ethynylpiperidine; (2S,5R)-2-(5-(1-carbamimidoylpiperidin-4-yl)isoxazol-3-yl)-7-oxo-1,6-diazabicyclo[3.2.1]octan-6-yl hydrogen sulfate TFA salt (607, 32 mg) was obtained. ESI-MS (EI⁺, m/z): 415.2.

### Example 10

### Synthesis of (2S,5R)-2-(5-((2-aminoethyl)carbamoyl)isoxazol-3-yl)-7-oxo-1,6-diazabicyclo[3.2.1]octan-6-yl hydrogen sulfate (Compound 605)

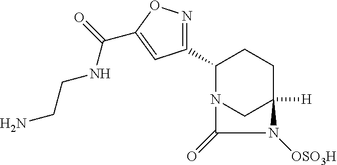

Following Steps 1-6 in Example 4, replacing tert-butyl prop-2-yn-1-ylcarbamate in Step 2 with tert-butyl (2-propiolamidoethyl)carbamate, (2S,5R)-2-(5-((2-aminoethyl)carbamoyl)isoxazol-3-yl)-7-oxo-1,6-diazabicyclo[3.2.1]octan-6-yl hydrogen sulfate TFA salt (605, 20 mg) was obtained as a light yellow solid. ESI-MS (EI⁺, m/z): 376.1. ¹H NMR (300 MHz, D₂O) δ 7.05 (s, 1H), 4.70 (m, 1H, overlapped with D₂O peak) 4.16 (br s, 1H), 3.66 (t, J=5.8 Hz, 2H), 3.19 (t, J=5.8 Hz, 2H), 3.16-3.08 (m, 1H), 2.93 (d, J=12.0 Hz, 1H), 2.31-2.24 (m, 1H), 2.16-2.04 (m, 2H), 1.94-1.85 (m, 1H).

### Example 11

### Synthesis of (2S,5R)-2-(5-((2-guanidinoethyl)carbamoyl)isoxazol-3-yl)-7-oxo-1,6-diazabicyclo[3.2.1]octan-6-yl hydrogen sulfate (Compound 608)

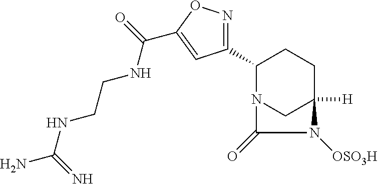

Following Steps 1-5 in Example 5, replacing prop-2-yn-1-amine in Step 1 with N-(2-aminoethyl)propiolamide; (2S,5R)-2-(5-((2-guanidinoethyl)carbamoyl)isoxazol-3-yl)-7-oxo-1,6-diazabicyclo[3.2.1]octan-6-yl hydrogen sulfate (608, 25 mg) was obtained as a white solid after prep-HPLC purification using ammonium formate buffer. ESI-MS (EI⁺, m/z): 418.12. ¹H NMR (300 MHz, D₂O) δ 4.79-4.76 (m, 1H), 4.16 (br s, 1H), 4.47-4.32 (m, 4H), 4.17-4.11 (m, 2H), 3.19-3.16 (m, 1H), 2.92 (d, J=12.0 Hz, 1H), 2.32-2.22 (m, 1H), 2.20-2.06 (m, 2H), 1.97-1.88 (m, 1H).

### Example 12

### Synthesis of (2S,5R)-7-oxo-2-(5-(2-(piperidin-4-ylamino)ethyl)isoxazol-3-yl)-1,6-diazabicyclo[3.2.1]octan-6-yl hydrogen sulfate (Compound 609)

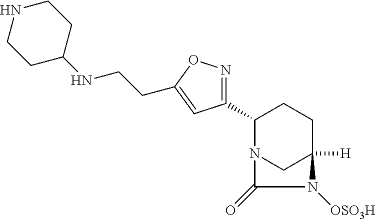

Synthetic Scheme:

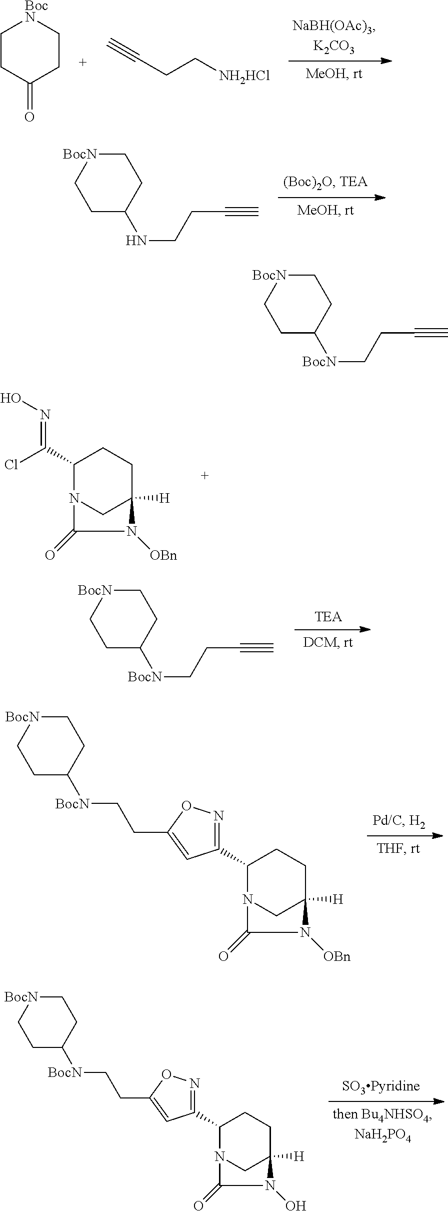

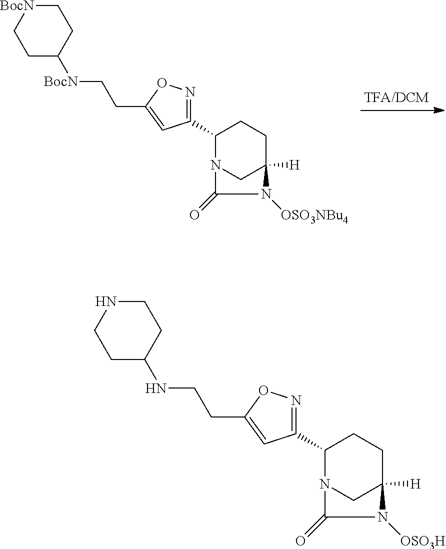

Procedures and Characterization:

Step 1: Synthesis of tert-butyl 4-(but-3-ynylamino)piperidine-1-carboxylate: A mixture of but-3-yn-1-amine hydrochloride (26.4 g, 0.25 mol), K₂CO₃ (17.4 g, 0.13 mol), and tert-butyl 4-oxopiperidine-1-carboxylate (41.8 g, 0.21 mol) in MeOH (500 mL) was stirred at rt for 5 hrs. Then, NaBH(OAc)₃ (133.6 g, 0.63 mol) was added and the suspension was stirred at rt for 17 hrs. The crude reaction mixture was used directly in the next step. ESI-MS (EI⁺, m/z): 253.2 [M+H]⁺.

Step 2: Synthesis of tert-butyl 4-(but-3-ynyl(tert-butoxycarbonyl)amino)piperidine-1-carboxylate:

The reaction mixture in Step 1 was cooled to 0° C. and Et₃N (88.0 mL, 0.63 mol) and (Boc)₂O (91.6 g, 0.42 mol) were added. The solution was allowed to warm to rt, then was stirred at rt for 17 hrs. The reaction mixture was concentrated, and then EtOAc (800 mL) was added. The organic layer was washed with saturated sodium chloride (3×), dried over Na₂SO₄, and concentrated. The residue was purified by silica gel column chromatography (gradient elution 0-10% EtOAC/petroleum ether) to afford tert-butyl 4-(but-3-ynyl(tert-butoxycarbonyl)amino)piperidine-1-carboxylate (66.0 g, 90%) as a colorless oil. ESI-MS (EI⁺, m/z): 353.2 [M+H]⁺.

Step 3-6: Following Steps 2-5 in Example 5, replacing 1-(2,3-bis(tert-butoxycarbonyl)guanidino)prop-2-yne in Step 2 with tert-butyl 4-(but-3-yn-1-yl(tert-butoxycarbonyl)amino)piperidine-1-carboxylate; (2S,5R)-7-oxo-2-(5-(2-(piperidin-4-ylamino)ethyl)isoxazol-3-yl)-1,6-diazabicyclo[3.2.1]octan-6-yl hydrogen sulfate (609, 220 mg) was obtained as a white solid after prep-HPLC purification using ammonium formate buffer. ESI-MS (EI⁺, m/z): 416.4. ¹H NMR (300 MHz, D₂O) δ 6.29 (s, 1H), 4.59 (d, J=6.3 Hz, 1H), 4.12 (s, 1H), 3.39 (d, J=13.3 Hz, 2H), 3.10-2.91 (m, 9H), 2.23-1.95 (m, 5H), 1.87-1.76 (m, 1H), 1.54-1.40 (m, 2H).

### Example 13

### Synthesis of (2S,5R)-2-(5-(2-((1-carbamimidoylpiperidin-4-yl)amino)ethyl)isoxazol-3-yl)-7-oxo-1,6-diazabicyclo[3.2.1]octan-6-yl hydrogen sulfate (Compound 610)

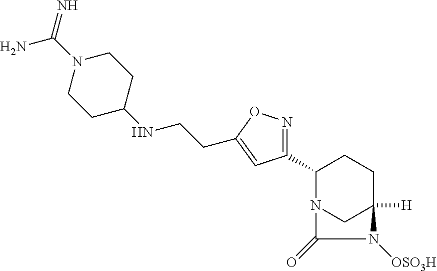

Synthetic Scheme:

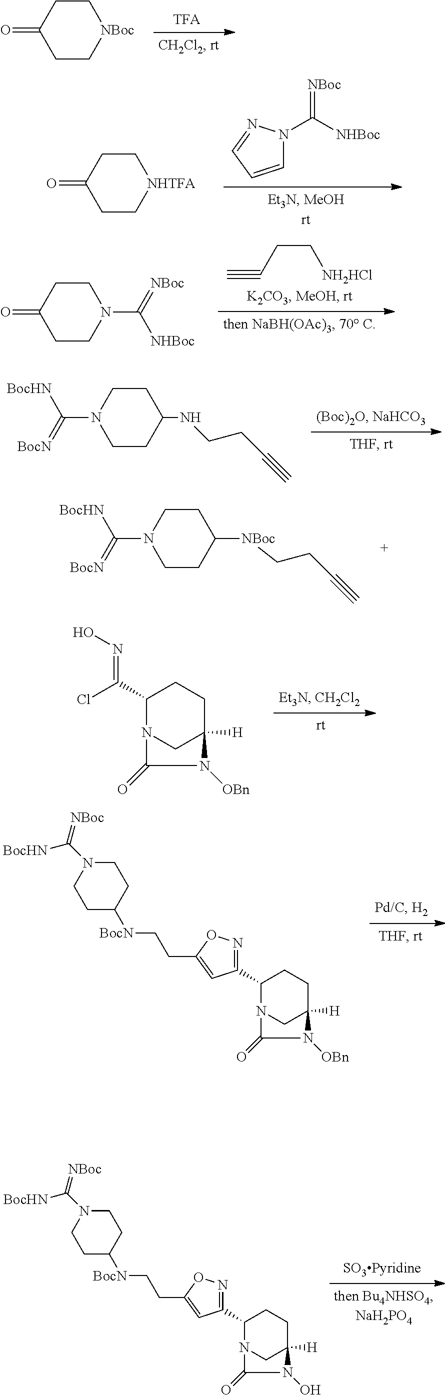

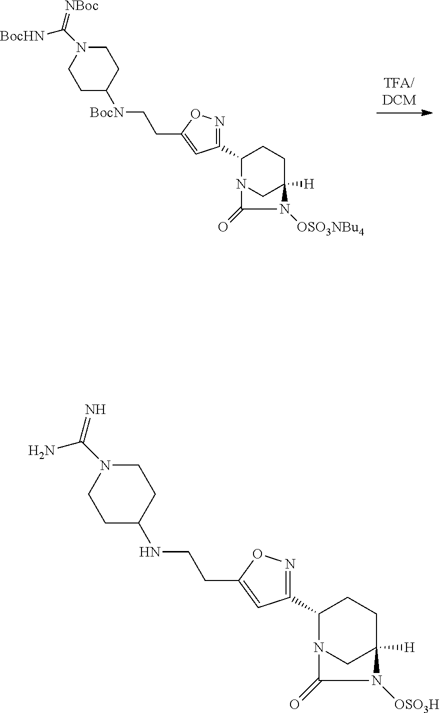

Procedures and Characterization:

Step 1: Synthesis of piperidin-4-one:

A mixture of tert-butyl 4-oxopiperidine-1-carboxylate (10.0 g, 50 mmol), TFA (20 mL) and CH₂Cl₂ (100 mL) was stirred at rt for 1 h. The reaction mixture was then concentrated to afford piperidin-4-one (15.0 g, 100%) as a colorless oil. ¹H NMR: (500 MHz, DMSO-d₆): δ 2.51 (m, 4H), 3.43 (m, 4H), 9.03 (m, 2H).

Step 2: Synthesis of di-tert-butyl (4-oxopiperidin-1-yl)methylenedicarbamate:

(Z)-di-tert-butyl (1H-imidazol-1-yl)methylenedicarbamate (16.3 g, 52.5 mmol) was add to a 0° C. solution of piperidin-4-one (15 g, 50 mmol) and Et₃N (25.4 g, 251 mmol) in MeOH (200 mL). The reaction mixture was allowed to warm to rt then was stirred at rt for 17 hrs. The reaction mixture was concentrated, and then EtOAc (300 mL) was added. The organic layer was washed with saturated sodium chloride (3×), dried over Na₂SO₄, and concentrated. The residue was purified by silica gel column chromatography (gradient elution 0-20% EtOAc/petroleum ether) to afford di-tert-butyl(4-oxopiperidin-1-yl)methylenedicarbamate (12.8 g, 75%) as a white solid. ESI-MS (EI⁺, m/z): 342.1 [M+H]⁺.

Step 3: Synthesis of di-tert-butyl(4-(but-3-ynylamino)piperidin-1-yl)methanediylidenedicarbamate:

A mixture of but-3-yn-1-amine hydrochloride (3.0 g, 28.4 mmol), K₂CO₃ (2.0 g, 14.2 mmol), MeOH (150 mL), di-tert-butyl (4-oxopiperidin-1-yl)methylene-dicarbamate (8.1 g, 23.7 mmol) and 4 Å molecular sieve (6.0 g) was stirred at rt for 3 hrs. Then, NaBH(OAc)₃ (15.1 g, 71.1 mmol) was added, and the suspension was stirred at 70° C. for 1 h. The reaction mixture was filtered and concentrated to afford di-tert-butyl(4-(but-3-ynylamino)piperidin-1-yl)methanediylidenedicarbamate as a yellow oil (˜15.0 g), which was used directly in the next step.

Step 4: Synthesis of di-tert-butyl(4-(but-3-ynyl(tert-butylcarbamate))piperidin-1-yl)methanedi-ylidenedicarbamate:

A mixture of di-tert-butyl(4-(but-3-ynylamino)piperidin-1-yl) methanediylidenedicarbamate (˜15.0 g), di-tert-butyl dicarbonate (10.3 g, 47.4 mmol), aq. NaHCO₃ (60 mL) in THF (120 mL) was stirred at rt for 2 hrs. The reaction mixture was then concentrated and extracted with EtOAc (3×). The combined organics were dried (Na₂SO₄) and concentrated. The residue was purified by silica gel column chromatography (gradient elution 0-20% EtOAc/petroleum ether) to afford di-tert-butyl(4-(but-3-ynyl(tert-butylcarbamate))piperidin-1-yl)methanedi-ylidenedicarbamate as a colorless oil (7.1 g, 60%). ESI-MS (EI⁺, m/z): 495.3 [M+H]⁺.

Step 5-8: Following Steps 2-5 in Example 5, replacing 1-(2,3-bis(tert-butoxycarbonyl)guanidino)prop-2-yne in Step 2 with tert-butyl (1-(N,N′-bis(tert-butoxycarbonyl)carbamimidoyl)piperidin-4-yl)(but-3-yn-1-yl)carbamate;(2S,5R)-2-(5-(2-((1-carbamimidoylpiperidin-4-yl)amino)ethyl)isoxazol-3-yl)-7-oxo-1,6-diazabicyclo[3.2.1]octan-6-yl hydrogen sulfate (610, 502 mg) was obtained as a white solid after prep-HPLC purification using ammonium formate buffer. ESI-MS (EI⁺, m/z): 458.0. ¹H NMR (300 MHz, D₂O) δ 6.35 (s, 1H), 4.59 (d, J=7.0 Hz, 1H), 4.12 (s, 1H), 3.85 (d, J=14.5 Hz, 2H), 3.40-3.30 (m, 3H), 3.20-2.90 (m, 6H), 2.20-1.75 (m, 6H), 1.62-1.46 (m, 2H).

The Compounds Described in Examples 14-33 were Prepared as Described in the Reaction Schemes Following Similar Procedures of Examples 1-13

### Example 14

### Synthesis of (2S,5R)-2-(5-((R*)-2-guanidino-1-hydroxyethyl)isoxazol-3-yl)-7-oxo-1,6-diazabicyclo[3.2.1]octan-6-yl hydrogen sulfate (Compound 611)

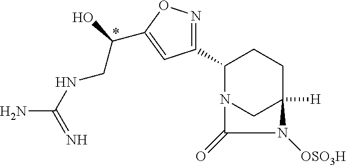

Synthetic Scheme:

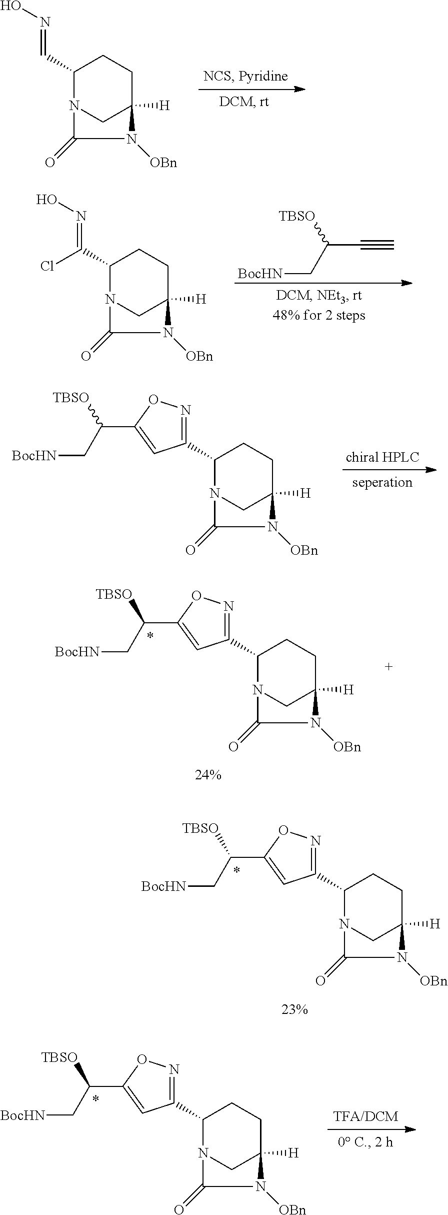

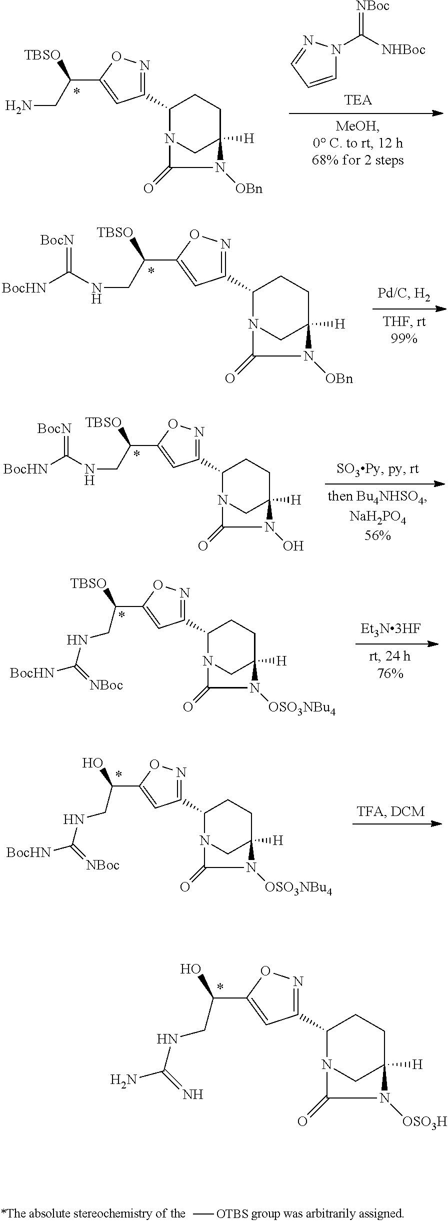

Characterization:

(2S,5R)-2-(5-((R*)-2-guanidino-1-hydroxyethyl)isoxazol-3-yl)-7-oxo-1,6-diazabicyclo[3.2.1]octan-6-yl hydrogen sulfate (611, 46 mg) was obtained as a white solid after prep-HPLC purification using ammonium formate buffer. ESI-MS (EI⁺, m/z): 391.0. ¹H NMR (300 MHz, D₂O) δ 6.46 (s, 1H), 5.04 (t, J=5.2 Hz, 1H), 4.62 (d, J=6.7 Hz, 1H), 4.11 (s, 1H), 3.56 (d, J=5.2 Hz, 2H), 3.08 (d, J=11.6 Hz, 1H), 2.85 (d, J=12.1 Hz, 1H), 2.24-2.14 (m, 1H), 2.12-2.00 (m, 2H), 1.86-1.76 (m, 1H).

### Example 15

### Synthesis of (2S,5R)-2-(5-((S*)-2-guanidino-1-hydroxyethyl)isoxazol-3-yl)-7-oxo-1,6-diazabicyclo[3.2.1]octan-6-yl hydrogen sulfate (Compound 612)

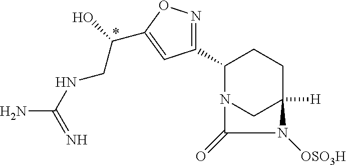

Synthetic Scheme:

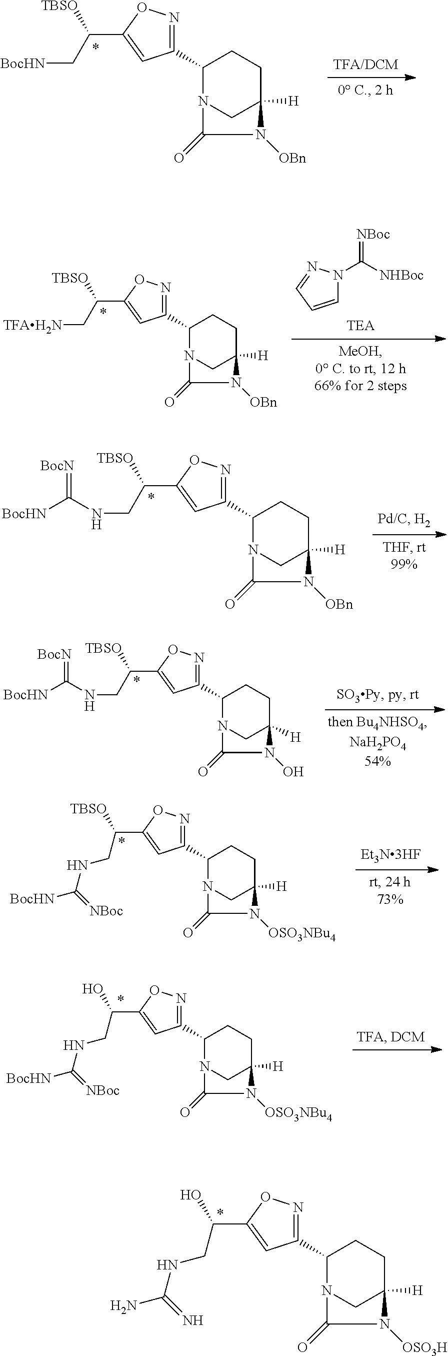

Characterization:

(2S,5R)-2-(5-((S*)-2-guanidino-1-hydroxyethyl)isoxazol-3-yl)-7-oxo-1,6-diazabicyclo[3.2.1]octan-6-yl hydrogen sulfate (612, 72 mg) was obtained as a white solid after prep-HPLC purification using ammonium formate buffer. ESI-MS (EI⁺, m/z): 391.0. ¹H NMR (300 MHz, D₂O) δ 6.47 (s, 1H), 5.04 (t, J=5.4 Hz, 1H), 4.63 (d, J=6.2 Hz, 1H), 4.12 (s, 1H), 3.65-3.49 (m, 2H), 3.09 (br d, J=12.7 Hz, 1H), 2.87 (d, J=12.2 Hz, 1H), 2.20-1.98 (m, 3H), 1.86-1.78 (m, 1H).

### Example 16

### Synthesis of (2S,5R)-2-(5-((1-guanidinocyclopropyl)methyl)isoxazol-3-yl)-7-oxo-1,6-diazabicyclo[3.2.1]octan-6-yl hydrogen sulfate (Compound 613)

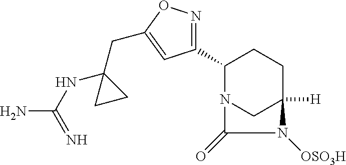

Synthetic Scheme:

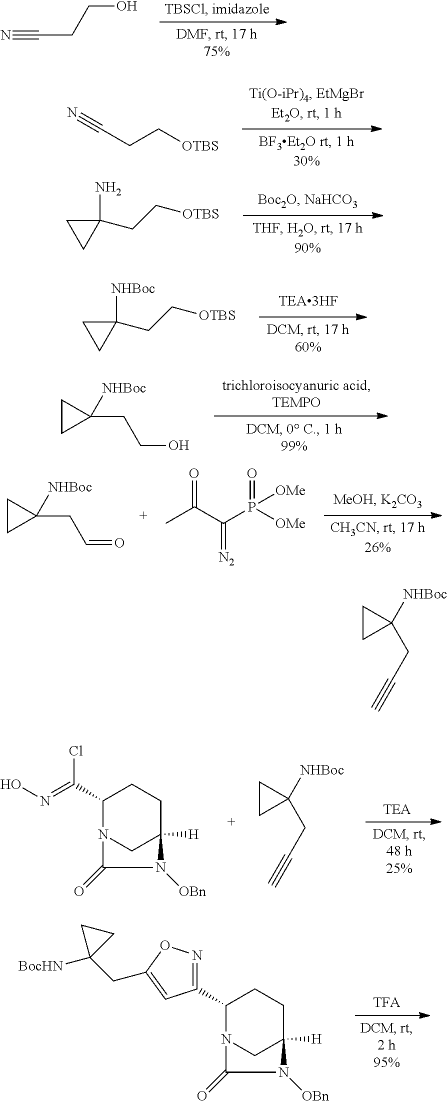

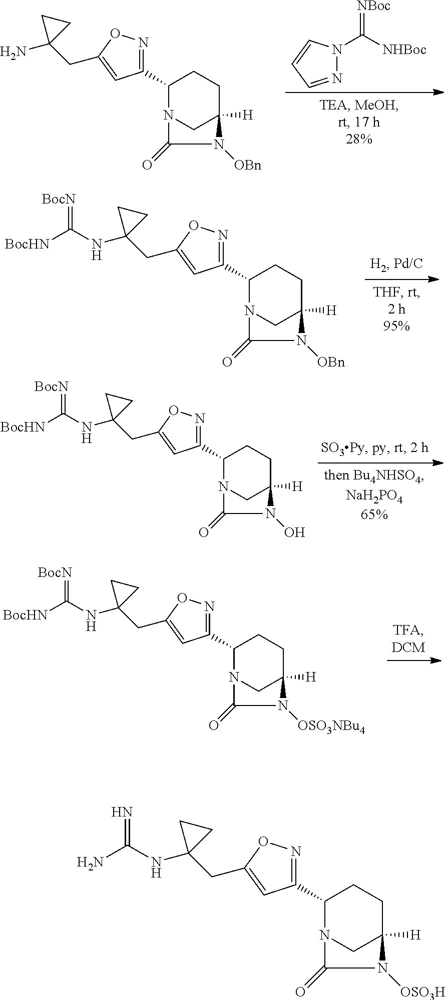

Characterization:

(2S,5R)-2-(5-((1-guanidinocyclopropyl)methyl)isoxazol-3-yl)-7-oxo-1,6-diazabicyclo[3.2.1]octan-6-yl hydrogen sulfate (613, 28 mg) was obtained as a white solid after prep-HPLC purification using ammonium formate buffer. ESI-MS (EI⁺, m/z): 401.3. ¹H NMR (300 MHz, D₂O) δ 6.35 (s, 1H), 4.61 (d, J=6.6 Hz, 1H), 4.12 (br s, 1H), 3.11-3.09 (m, 1H), 3.08-2.98 (m, 2H), 2.89 (d, J=12.0 Hz, 1H), 2.20-2.00 (m, 3H), 1.89-1.78 (m, 1H), 0.97-0.95 (m, 4H).

### Example 17

### Synthesis of (2S,5R)-2-(5-((1-methylguanidino)methyl)isoxazol-3-yl)-7-oxo-1,6-diazabicyclo[3.2.1]octan-6-yl hydrogen sulfate (Compound 614)

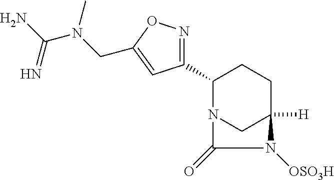

Synthetic Scheme:

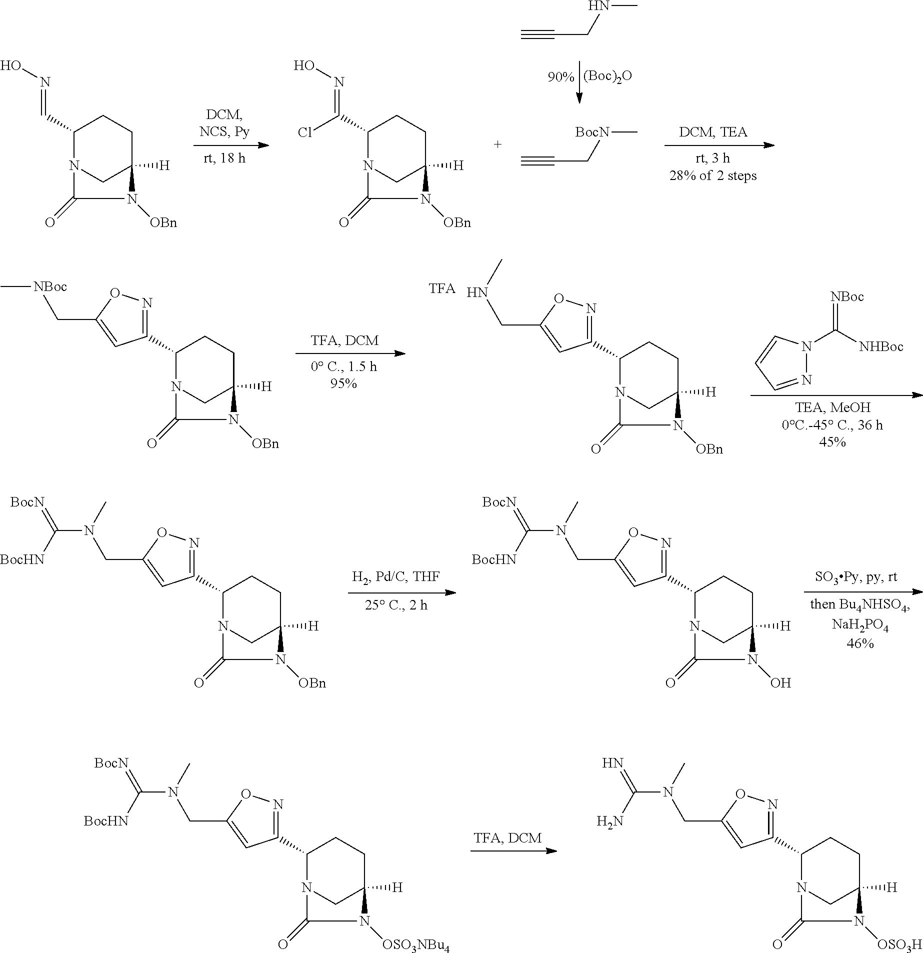

Characterization:

(2S,5R)-2-(5-((1-methylguanidino)methyl)isoxazol-3-yl)-7-oxo-1,6-diazabicyclo[3.2.1]octan-6-yl hydrogen sulfate (614, 12 mg) was obtained as a white solid after prep-HPLC purification using ammonium formate buffer. ESI-MS (EI⁺, m/z): 375.1. ¹H NMR (300 MHz, D₂O with coupe of drops of DMSO-d6) δ 6.55 (s, 1H), 4.73 (s, 2H), 4.64 (d, J=6.0 Hz, 1H), 4.14 (br s, 1H), 3.15-3.07 (m, 1H), 3.06 (s, 3H), 2.89 (d, J=12.2 Hz, 1H), 2.26-2.20 (m, 1H), 2.15-2.09 (m, 2H), 1.92-1.85 (m, 1H).

### Example 18

### Synthesis of (2S,5R)-2-(5-(3-guanidinopropyl)isoxazol-3-yl)-7-oxo-1,6-diazabicyclo[3.2.1]octan-6-yl hydrogen sulfate (Compound 615)

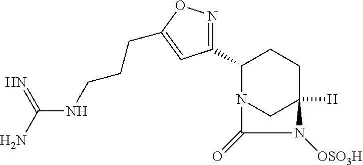

Synthetic Scheme:

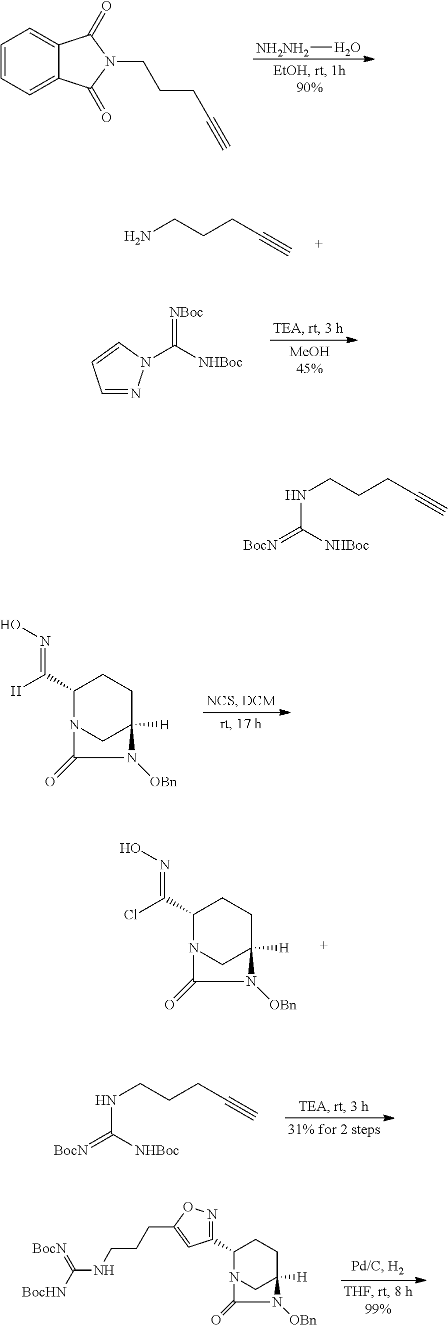

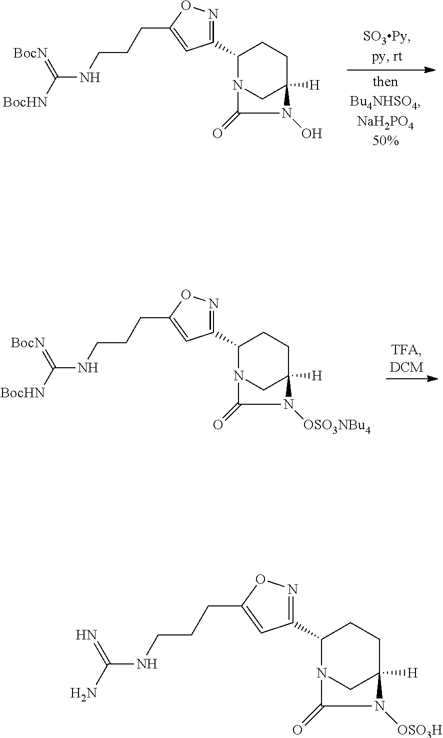

Characterization:

(2S,5R)-2-(5-(3-guanidinopropyl)isoxazol-3-yl)-7-oxo-1,6-diazabicyclo[3.2.1]octan-6-yl hydrogen sulfate (615, 18 mg) was obtained as a white solid after prep-HPLC purification using ammonium formate buffer. ESI-MS (EI⁺, m/z): 389.1. ¹H NMR (300 MHz, D₂O with couple of drops of DMSO-d6) δ 6.28 (s, 1H), 4.57 (d, J=6.5 Hz, 1H), 4.11 (s, 1H), 3.17 (t, J=6.7 Hz, 2H), 3.07 (br d, J=11.5 Hz, 1H), 2.88 (d, J=12.4 Hz, 1H), 2.83 (t, J=6.7 Hz, 2H), 2.22-1.75 (m, 6H).

### Example 19

### Synthesis of (2S,5R)-2-(5-((1s,3R)-3-aminocyclobutyl)isoxazol-3-yl)-7-oxo-1,6-diazabicyclo[3.2.1]octan-6-yl hydrogen sulfate (Compound 616)

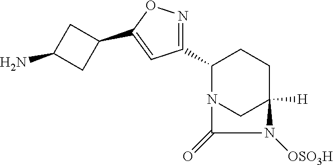

Synthetic Scheme:

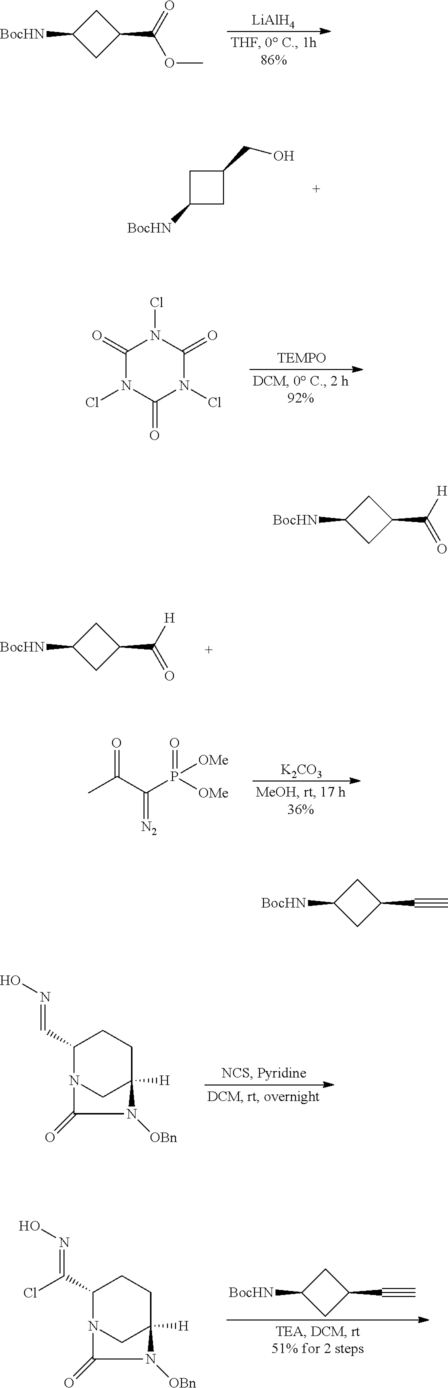

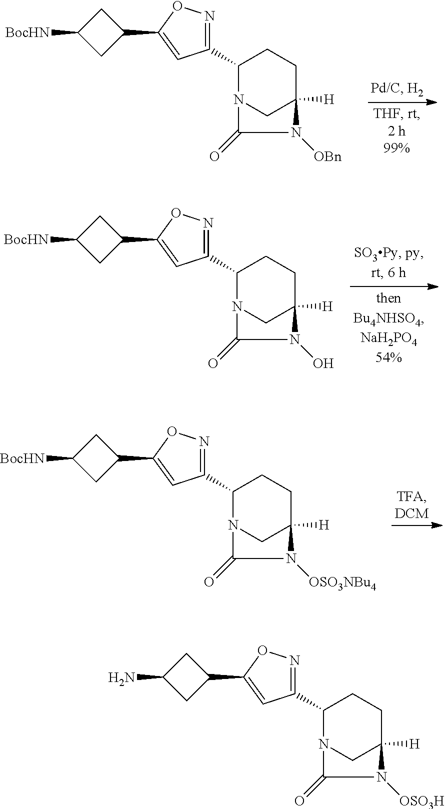

Characterization:

(2S,5R)-2-(5-((1s,3R)-3-aminocyclobutyl)isoxazol-3-yl)-7-oxo-1,6-diazabicyclo[3.2.1]octan-6-yl hydrogen sulfate (616, 7 mg) was obtained as a white solid after prep-HPLC purification using ammonium formate buffer. ESI-MS (EI⁺, m/z): 359.0. ¹H NMR (300 MHz, DMSO) δ 6.43 (s, 1H), 4.46 (d, J=6.1 Hz, 1H), 4.02 (br s, 1H), 3.75-3.68 (m, 1H), 3.51-3.45 (m, 1H), 2.94-2.85 (m, 1H), 2.72-2.60 (m, 3H), 2.36-2.30 (m, 2H), 2.15-1.95 (m, 1H), 1.99-1.90 (m, 2H), 1.78-1.72 (m, 1H).

### Example 20

### Synthesis of (2S,5R)-2-(5-((1s,3R)-3-guanidinocyclobutyl)isoxazol-3-yl)-7-oxo-1,6-diazabicyclo[3.2.1]octan-6-yl hydrogen sulfate (Compound 617)

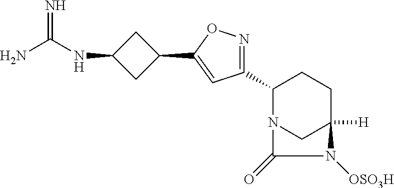

Synthetic Scheme:

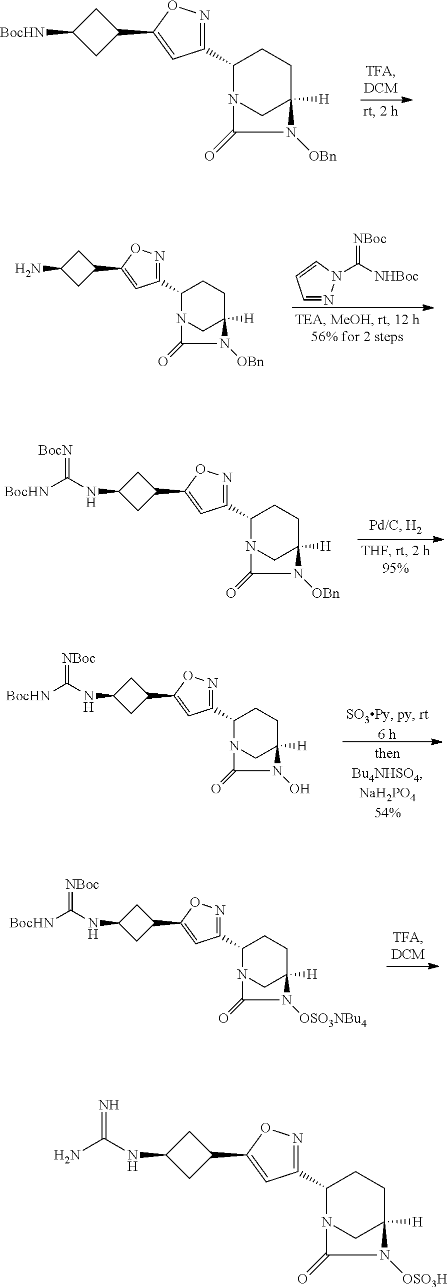

Characterization:

(2S,5R)-2-(5-((1s,3R)-3-guanidinocyclobutyl)isoxazol-3-yl)-7-oxo-1,6-diazabicyclo[3.2.1]octan-6-yl hydrogen sulfate (617, 37 mg) was obtained as a white solid after prep-HPLC purification using ammonium formate buffer. ESI-MS (EI⁺, m/z): 401.0. ¹H NMR (300 MHz, DMSO) δ 8.11 (br s, 1H), 7.14 (br s, 4H), 6.41 (s, 1H), 4.45 (d, J=6.3 Hz, 1H), 4.02 (br s, 2H), 3.40-3.30 (m, 1H), 2.91 (br d, J=10.3 Hz, 1H), 2.80-2.63 (m, 3H), 2.30-2.10 (m, 3H), 2.00-1.85 (m, 2H), 1.82-1.66 (m, 1H).

### Example 21

### Synthesis of (2S,5R)-2-(5-(azetidin-3-ylmethyl)isoxazol-3-yl)-7-oxo-1,6-diazabicyclo[3.2.1]octan-6-yl hydrogen sulfate (Compound 618,)

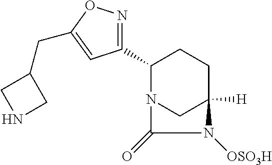

Synthetic Scheme:

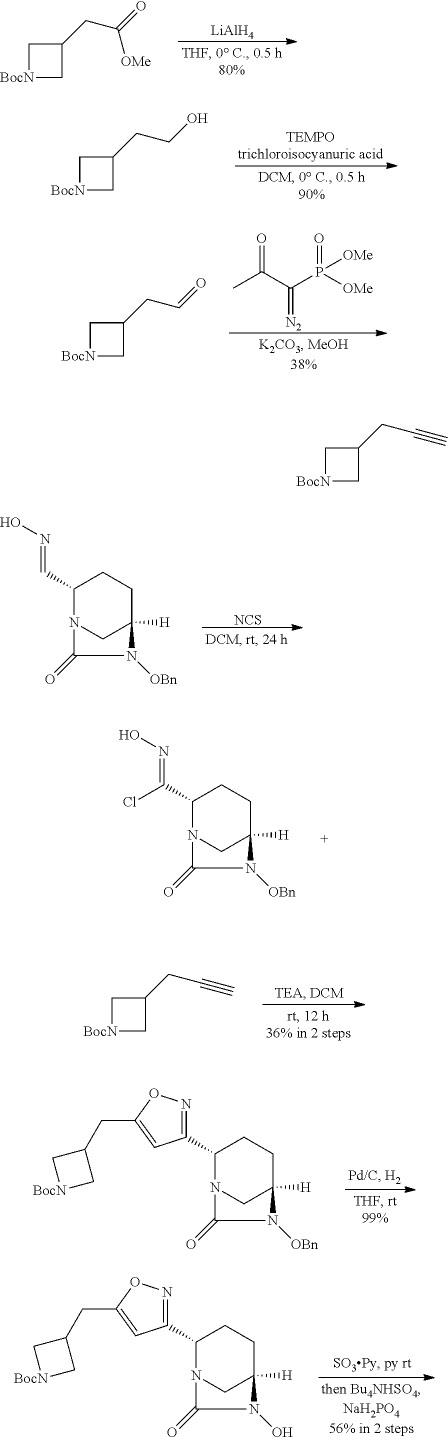

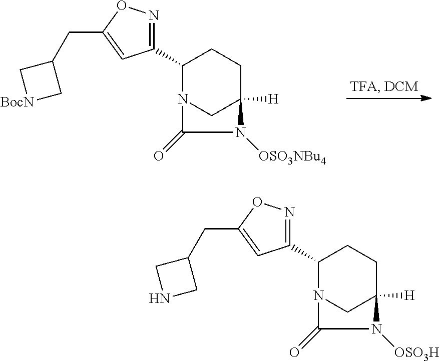

Characterization:

(2S,5R)-2-(5-(azetidin-3-ylmethyl)isoxazol-3-yl)-7-oxo-1,6-diazabicyclo[3.2.1]octan-6-yl hydrogen sulfate (618, 75 mg) was obtained as a white solid after prep-HPLC purification using ammonium formate buffer. ESI-MS (EI⁺, m/z): 359.0. ¹H NMR (300 MHz, D₂O) δ 6.23 (s, 1H), 4.56 (d, J=6.6 Hz, 1H), 4.15-4.08 (m, 2H), 3.93-3.82 (m, 2H), 3.44 (dd, J=14.3, 7.2 Hz, 1H), 3.28 (dt, J=15.9, 7.9 Hz, 1H), 3.10-3.03 (m, 3H), 2.87 (d, J=12.1 Hz, 1H), 2.21-1.94 (m, 3H), 1.84-1.76 (m, 1H).

### Example 22

### Synthesis of (2S,5R)-2-(5-((1-carbamimidoylazetidin-3-yl)methyl)isoxazol-3-yl)-7-oxo-1,6-diazabicyclo[3.2.1]octan-6-yl hydrogen sulfate (Compound 619,)

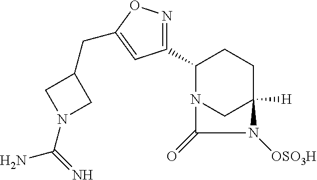

Synthetic Scheme:

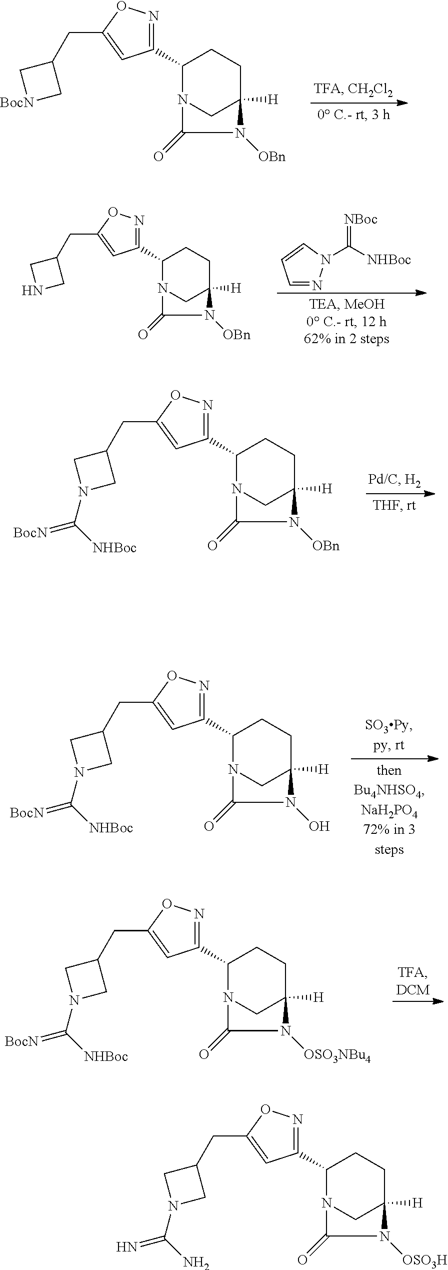

Characterization:

(2S,5R)-2-(5-((1-carbamimidoylazetidin-3-yl)methyl)isoxazol-3-yl)-7-oxo-1,6-diazabicyclo[3.2.1]octan-6-yl hydrogen sulfate (619, 45 mg) was obtained as a white solid after prep-HPLC purification using ammonium formate buffer. ESI-MS (EI⁺, m/z): 401.0. ¹H NMR (300 MHz, DMSO) δ 7.28 (br s, 4H), 6.36 (s, 1H), 4.45 (d, J=6.8 Hz, 1H), 4.18 (t, J=8.4 Hz, 2H), 4.02 (br s, 1H), 3.86-3.71 (m, 2H), 3.16 (d, J=7.0 Hz, 2H), 3.09-3.04 (m, 1H), 2.90 (br d, J=8.7 Hz, 1H), 2.67 (d, J=11.8 Hz, 1H), 2.16-2.09 (m, 1H), 2.00-1.90 (m, 2H), 1.80-1.72 (m, 1H).

### Example 23

### Synthesis of (2S,5R)-2-(5-(2-((2-aminoethyl)amino)ethyl)isoxazol-3-yl)-7-oxo-1,6-diazabicyclo[3.2.1]octan-6-yl hydrogen sulfate (Compound 620)

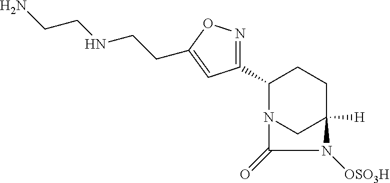

Synthetic Scheme:

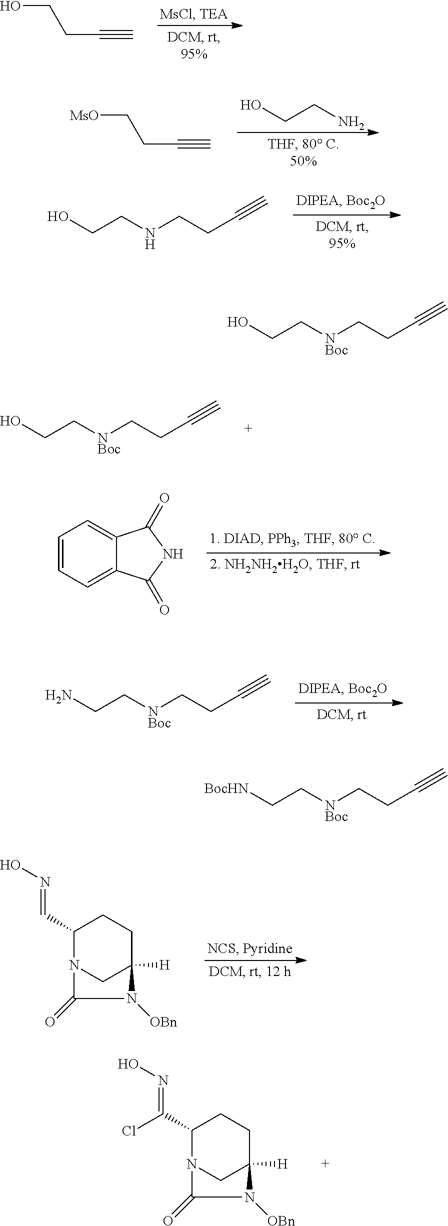

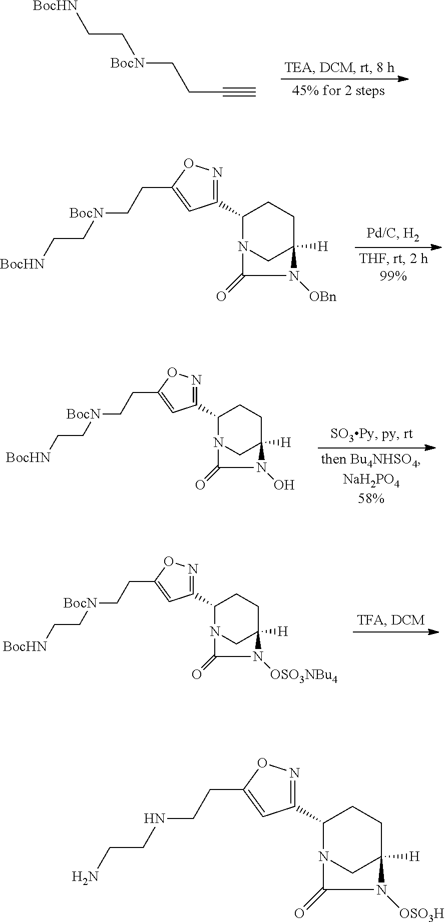

Characterization:

(2S,5R)-2-(5-(2-((2-aminoethyl)amino)ethyl)isoxazol-3-yl)-7-oxo-1,6-diazabicyclo[3.2.1]octan-6-yl hydrogen sulfate (620, 20 mg, as formate salt) was obtained as a white solid after prep-HPLC purification using ammonium formate buffer. ESI-MS (EI⁺, m/z): 376.3. ¹H NMR (300 MHz, D₂O) δ 8.32 (s, 1H), 6.35 (s, 1H), 4.59 (d, J=6.5 Hz, 1H), 4.11 (br s, 1H), 3.34 (t, J=6.8 Hz, 2H), 3.30-3.13 (m, 6H), 3.11-3.03 (m, 1H), 2.91 (d, J=12.2 Hz, 1H), 2.21-1.98 (m, 3H), 1.88-1.78 (m, 1H).

### Example 24

### Synthesis of (2S,5R)-2-(5-(2-((2-guanidinoethyl)amino)ethyl)isoxazol-3-yl)-7-oxo-1,6-diazabicyclo[3.2.1]octan-6-yl hydrogen sulfate (Compound 621,)

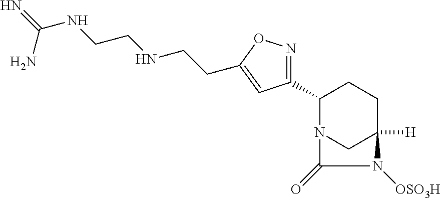

Synthetic Scheme:

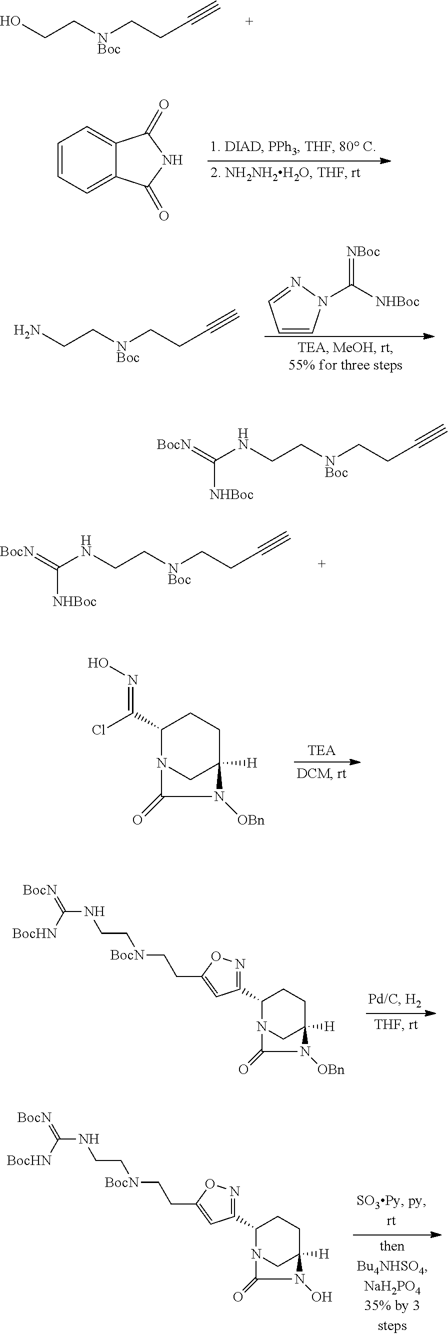

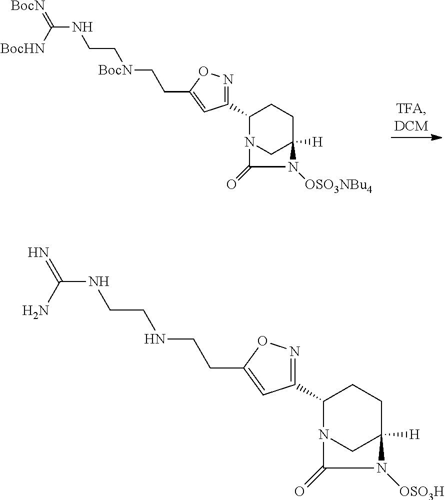

Characterization:

(2S,5R)-2-(5-(2-((2-guanidinoethyl)amino)ethyl)isoxazol-3-yl)-7-oxo-1,6-diazabicyclo[3.2.1]octan-6-yl hydrogen sulfate (612, 15 mg, as formate salt) was obtained as a white solid after prep-HPLC purification using ammonium formate buffer. ESI-MS (EI⁺, m/z): 418.0. ¹H NMR (300 MHz, D₂O) δ 8.28 (s, 1H), 6.30 (s, 1H), 4.55 (d, J=5.9 Hz, 1H), 4.07 (br s, 1H), 3.40 (t, J=5.9 Hz, 2H), 3.25 (t, J=6.1 Hz, 2H), 3.14-3.01 (m, 5H), 2.87 (d, J=12.1 Hz, 1H), 2.16-2.09 (m, 1H), 2.07-1.92 (m, 2H), 1.83-1.74 (m, 1H).

### Example 25

### Synthesis of (2S,5R)-2-(5-((azetidin-3-ylamino)methyl)isoxazol-3-yl)-7-oxo-1,6-diazabicyclo[3.2.1 ]octan-6-yl hydrogen sulfate (Compound 622,)

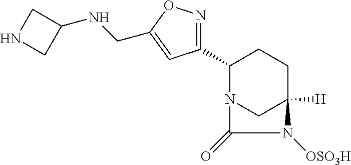

Synthetic Scheme:

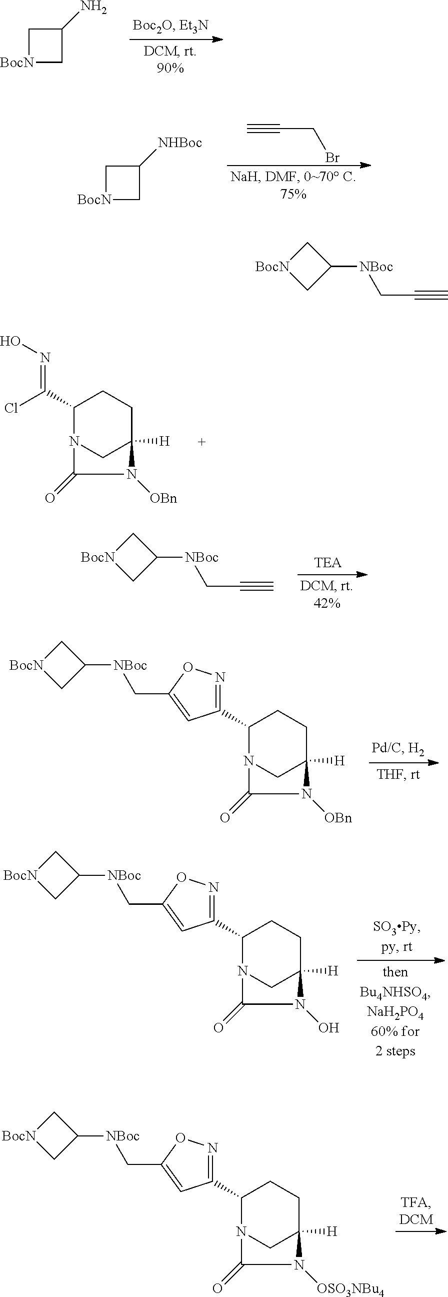

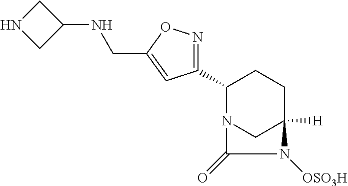

Characterization:

(2S,5R)-2-(5-((azetidin-3-ylamino)methyl)isoxazol-3-yl)-7-oxo-1,6-diazabicyclo[3.2.1]octan-6-yl hydrogen sulfate (622, 267 mg) was obtained as a white solid after prep-HPLC purification using ammonium formate buffer. ESI-MS (EI⁺, m/z): 374.0. ¹H NMR (400 MHz, D₂O) δ 6.55 (s, 1H), 4.66 (d, J=6.6 Hz, 1H), 4.29-4.10 (m, 5H), 4.10-3.98 (m, 2H), 3.12 (d, J=12.0 Hz, 1H), 2.91 (d, J=12.1 Hz, 1H), 2.30-2.18 (m, 1H), 2.18-1.99 (m, 2H), 1.97-1.78 (m, 1H).

### Example 26

### Synthesis of (2S,5R)-2-(5-(2-(azetidin-3-ylamino)ethyl)isoxazol-3-yl)-7-oxo-1,6-diazabicyclo[3.2.1]octan-6-yl hydrogen sulfate (Compound 623,)

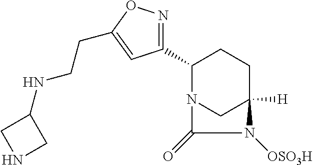

Synthetic Scheme:

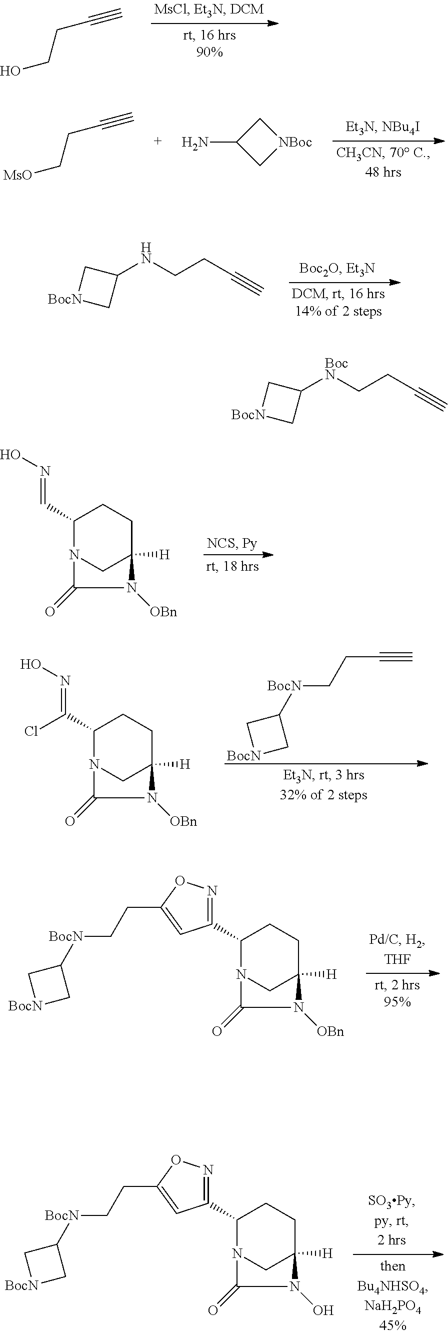

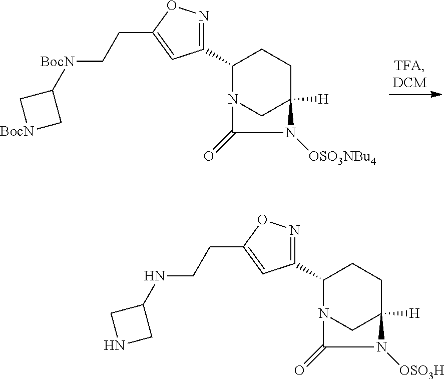

Characterization:

(2S,5R)-2-(5-(2-(azetidin-3-ylamino)ethyl)isoxazol-3-yl)-7-oxo-1,6-diazabicyclo[3.2.1]octan-6-yl hydrogen sulfate (623, 41 mg) was obtained as a white solid after prep-HPLC purification using ammonium formate buffer. ESI-MS (EI⁺, m/z): 388.0.

### Example 27

### Synthesis of (2S,5R)-2-(5-((azetidin-3-yloxy)methyl)isoxazol-3-yl)-7-oxo-1,6-diazabicyclo[3.2.1]octan-6-yl hydrogen sulfate (Compound 624)

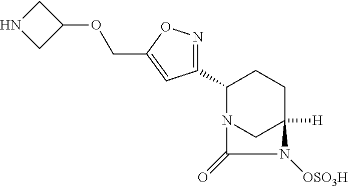

Synthetic Scheme:

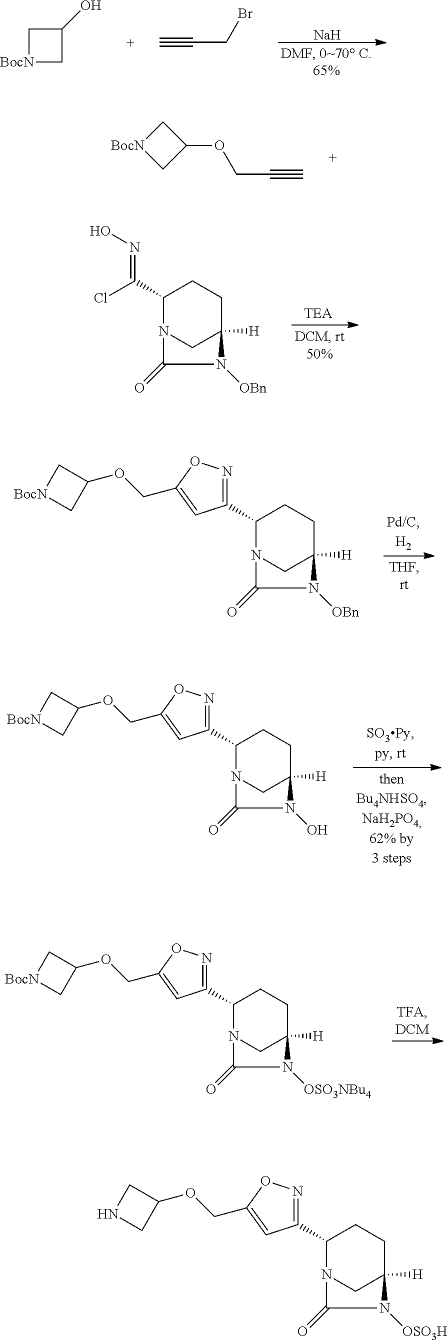

Characterization:

(2S,5R)-2-(5-((azetidin-3-yloxy)methyl)isoxazol-3-yl)-7-oxo-1,6-diazabicyclo[3.2.1]octan-6-yl hydrogen sulfate (624, 349 mg) was obtained as a white solid after prep-HPLC purification using ammonium formate buffer. ESI-MS (EI⁺, m/z): 375.2. ¹H NMR (300 MHz, D₂O) δ 6.51 (s, 1H), 4.64 (s, 2H), 4.62 (d, J=7.2 Hz, 1H), 4.56-4.52 (m, 1H), 4.20 (dd, J=12.5, 6.7 Hz, 2H), 4.11 (br s, 1H), 3.93 (dd, J=12.3, 5.2 Hz, 2H), 3.08 (br d, J=12.4 Hz, 1H), 2.87 (d, J=12.2 Hz, 1H), 2.22-2.14 (m, 1H), 2.12-2.00 (m, 2H), 187-1.78 (m, 1H).

### Example 28

### Synthesis of (2S,5R)-2-(5-(1-methylpyridin-1-ium-4-yl)isoxazol-3-yl)-7-oxo-1,6-diazabicyclo[3.2.1]octan-6-yl sulfate (Compound 625)

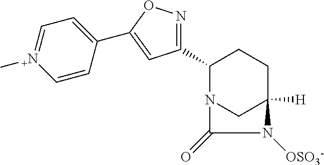

Synthetic Scheme:

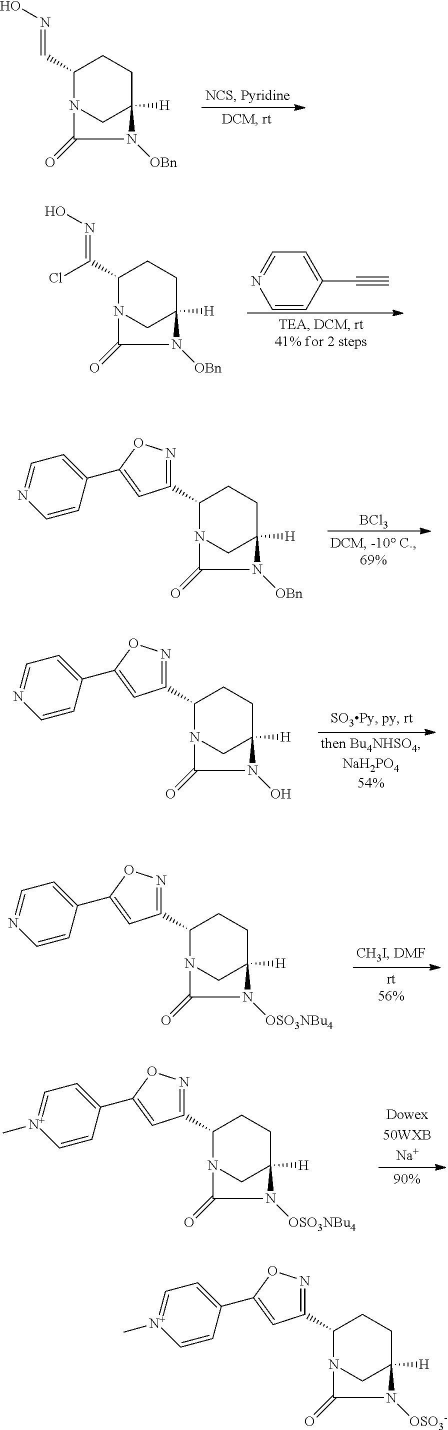

Characterization:

(2S,5R)-2-(5-(1-methylpyridin-1-ium-4-yl)isoxazol-3-yl)-7-oxo-1,6-diazabicyclo[3.2.1]octan-6-yl sulfate (625, 20 mg) was obtained as a white solid. ESI-MS (EI⁻, m/z): 379.0. ¹H NMR (400 MHz, d6-DMSO) δ 9.15 (d, J=5.2 Hz, 2H), 8.60 (d, J=5.2 Hz, 2H), 7.86 (s, 1H), 4.66 (d, J=5.6 Hz, 1H), 4.36 (s, 3H), 4.07 (br s, 1H), 3.00-2.98 (m, 1H), 2.79 (d, J=12.2 Hz, 1H), 2.24-2.20 (m, 1H), 2.10-2.00 (m, 2H), 1.82-1.78 (m, 1H).

### Example 29

### Synthesis of (2S,5R)-2-(5-(1,1-dimethylpiperidin-1-ium-4-yl)isoxazol-3-yl)-7-oxo-1,6-diazabicyclo[3.2.1]octan-6-yl sulfate (Compound 626)

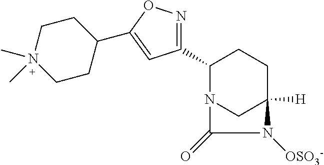

Synthetic Scheme:

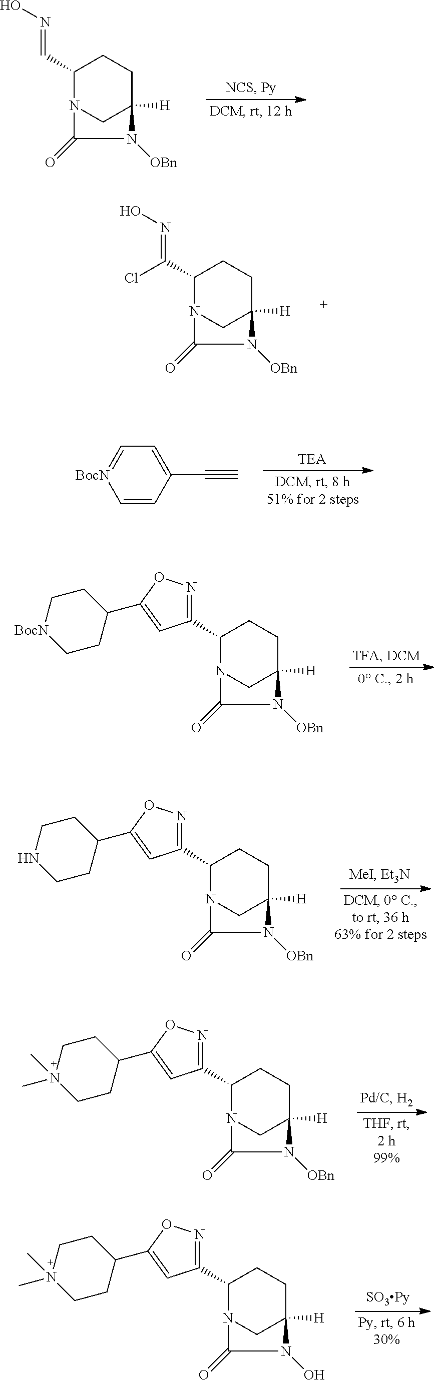

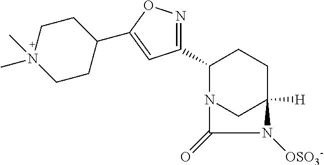

Characterization:

(2S,5R)-2-(5-(1,1-dimethylpiperidin-1-ium-4-yl)isoxazol-3-yl)-7-oxo-1,6-diazabicyclo[3.2.1]octan-6-yl sulfate (626, 86 mg) was obtained as a white solid after prep-HPLC purification using ammonium formate buffer. ESI-MS (EI⁻, m/z): 399.2. ¹H NMR (400 MHz, CF₃COOD): δ 7.24 (s, 1H), 6.03 (d, J=6.8 Hz, 1H), 5.44 (br s, 1H), 4.76-4.72 (m, 1H), 4.51 (d, J=12.2 Hz, 1H), 4.26-4.12 (m, 4H), 3.99-3.92 (m, 1H), 3.85 (s, 3H), 3.82 (s, 3H), 3.26-3.22 (m, 1H), 3.21-2.85 (m, 7H).

### Example 30

### Synthesis of (2S,5R)-7-oxo-2-(5-(piperidin-4-ylmethyl)isoxazol-3-yl)-1,6-diazabicyclo[3.2.1]octan-6-yl hydrogen sulfate (Compound 627)

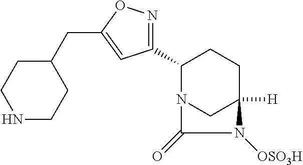

Synthetic Scheme:

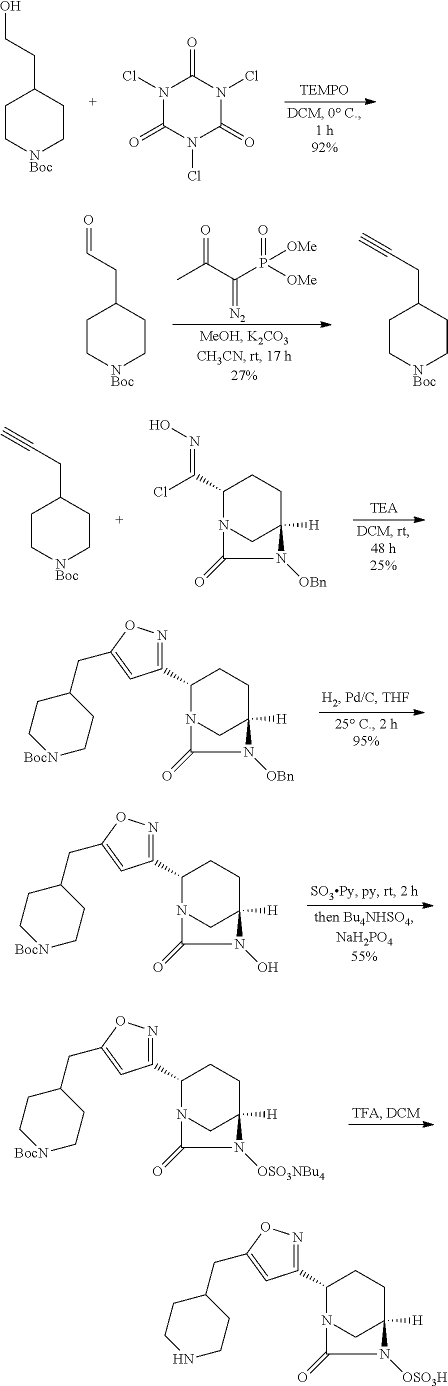

Characterization:

(2S,5R)-7-oxo-2-(5-(piperidin-4-ylmethyl)isoxazol-3-yl)-1,6-diazabicyclo[3.2.1]octan-6-yl hydrogen sulfate (627, 31 mg) was obtained as a white solid after prep-HPLC purification using ammonium formate buffer. ESI-MS (EI⁺, m/z): 387.0.

### Example 31

### Synthesis of (2S,5R)-2-(5-(azetidin-3-yl)isoxazol-3-yl)-7-oxo-1,6-diazabicyclo[3.2.1]octan-6-yl hydrogen sulfate (Compound 628)

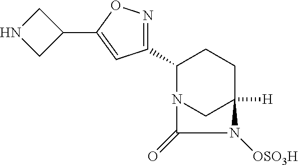

Characterization:

(2S,5R)-2-(5-(azetidin-3-yl)isoxazol-3-yl)-7-oxo-1,6-diazabicyclo[3.2.1]octan-6-yl hydrogen sulfate (628, 15 mg) was obtained as a white solid after prep-HPLC purification using ammonium formate buffer. ESI-MS (EI⁺, m/z): 345.0. ¹H NMR (300 MHz, D₂O) δ 6.48 (s, 1H), 4.62 (d, J=5.2 Hz, 1H), 4.47-4.22 (m, 5H), 4.15-4.12 (m, 1H), 3.17-3.03 (m, 1H), 2.91 (d, J=12.0 Hz, 1H), 2.24-2.17 (m, 1H), 2.12-2.00 (m, 2H), 1.90-1.80 (m, 1H). CB-606,122.

### Example 32

### Synthesis of (2S,5R)-2-(5-(2-(azetidin-3-yl)ethyl)isoxazol-3-yl)-7-oxo-1,6-diazabicyclo[3.2.1]octan-6-yl hydrogen sulfate (Compound 629)

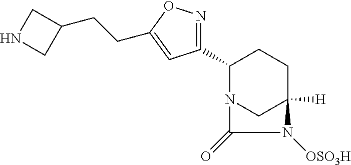

Synthetic Scheme:

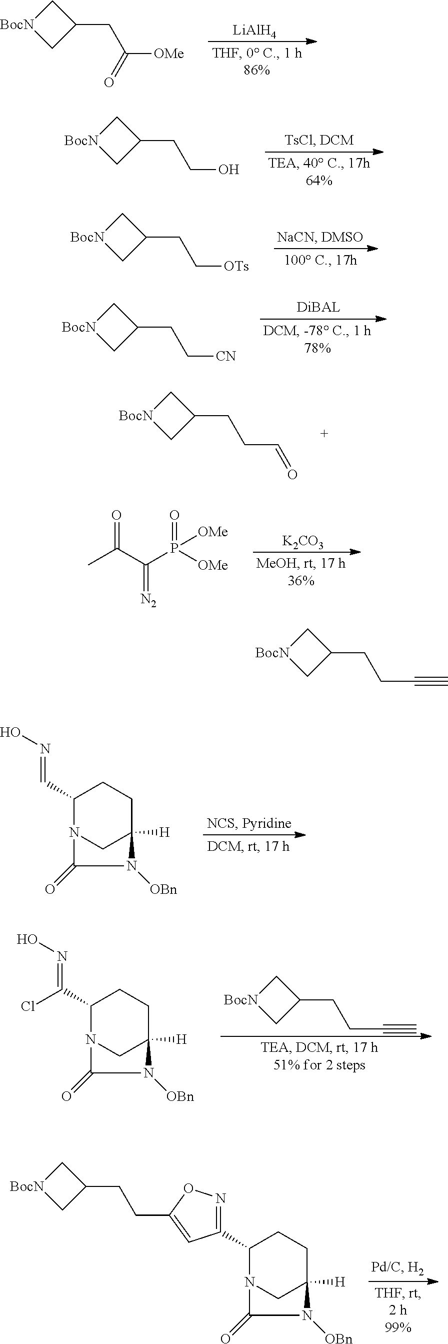

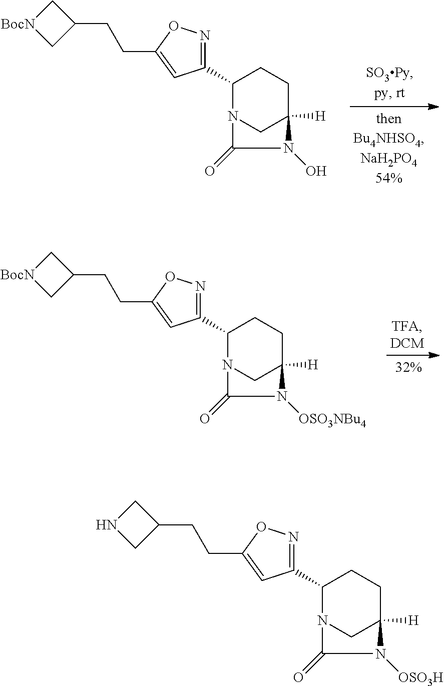

Characterization:

(2S,5R)-2-(5-(2-(azetidin-3-yl)ethyl)isoxazol-3-yl)-7-oxo-1,6-diazabicyclo[3.2.1]octan-6-yl hydrogen sulfate (629, 34 mg) was obtained as a white solid after prep-HPLC purification using ammonium formate buffer. ESI-MS (EI⁺, m/z): 373.0. ¹H NMR (300 MHz, DMSO) δ 8.33 (br s, 2H), 6.33 (s, 1H), 4.45 (d, J=6.4 Hz, 1H), 4.01 (br s, 1H), 3.92 (t, J=9.0 Hz, 2H), 3.61 (t, J=9.0 Hz, 2H), 3.40-3.26 (m, 1H), 2.94-2.88 (m, 1H), 2.85-2.70 (m, 4H), 2.16-2.08 (m, 1H), 2.06-1.87 (m, 3H), 1.84-1.67 (m, 1H).

### Example 33

### Synthesis of (2S,5R)-7-oxo-2-(5-(pyrrolidin-3-ylmethyl)isoxazol-3-yl)-1,6-diazabicyclo[3.2.1]octan-6-yl hydrogen sulfate (Compound 630)

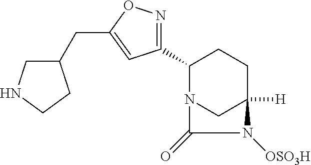

Synthetic Scheme:

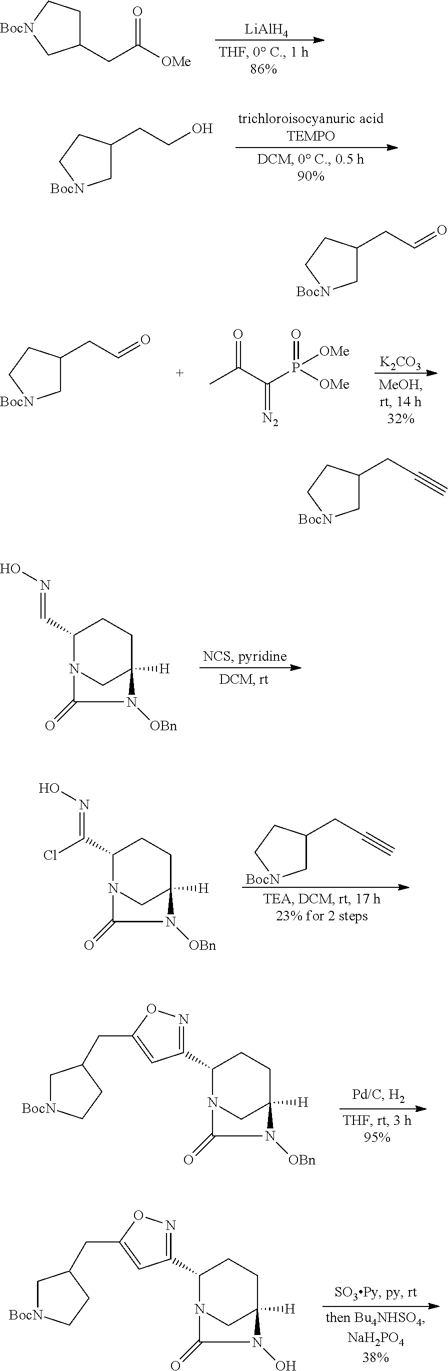

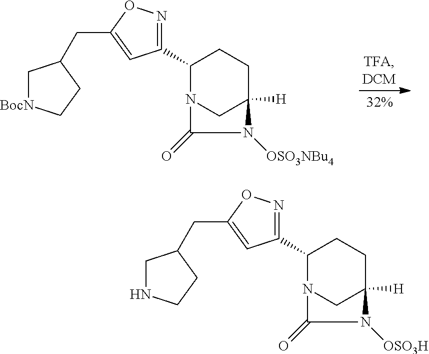

Characterization:

(2S,5R)-7-oxo-2-(5-(pyrrolidin-3-ylmethyl)isoxazol-3-yl)-1,6-diazabicyclo[3.2.1]octan-6-yl hydrogen sulfate (630, 9 mg) was obtained as a white solid after prep-HPLC purification using ammonium formate buffer. ESI-MS (EI⁻, m/z): 371.0. ¹H NMR (300 MHz, D₂O) δ 6.29 (s, 1H), 4.59 (d, J=6.6 Hz, 1H), 4.12 (br s, 1H), 3.43 (dd, J=7.6, 5.0 Hz, 1H), 3.39-3.29 (m, 1H), 3.26-3.14 (m, 1H), 3.12-3.03 (m, 1H), 2.97-2.85 (m, 4H), 2.74-2.61 (m, 1H), 2.20-2.00 (m, 4H), 1.87-1.78 (m, 1H), 1.74-1.60 (m, 1H).

### Example 34

### Construction of Isogenic β-Lactamase Strains

A set of β-lactamase expressing isogenic E. coli strains was constructed by cloning a β-lactamase gene into a customized derivative of pBR322 (GenBank Accession Number J01749) and transforming the engineered plasmids into E. coli. The NdeI restriction site within the plasmid backbone of pBR322 was removed to generate pBR322 ΔNdeI. The pBR322 ΔNdeI vector itself, minus the blaTEM-1 gene, was amplified using two primers: (1) pBR-Pbla 5′-cgcatatgactcttcctttttcaatattattg-3, SEQ ID 1, a primer with an engineered NdeI restriction site at the 3′ end of the blaTEM-1 promoter and (2) pBR-vec-1 5′-gcggatccctgtcagaccaagtttactc-3′, SEQ ID 2, a primer with an engineered BamHI restriction site at the 3′ end of the blaTEM-1 open reading frame. The chloramphenicol resistance gene, cat, was generated by PCR amplification from pKD3 (GenBank Accession Number AY048742) using primers with an engineered NdeI restriction site at the 5′ end (Pbla-cat 5′-gccatatgatggagaaaaaaatcactgg-3′, SEQ ID 3) and an engineered BamHI restriction site at the 3′ end (Vec-1-cat 5′-cgggatccctagagaataggaacttcgg-3′, SEQ ID 4)of the resistance gene. The two PCR products, pBR322 ΔNdeI and cat were ligated together generating pBR-CBST (pBR322 ΔNdeI ΔTEM-1::cat Seq. ID 5) which retains both the pBR322 tetracycline resistance cassette, tetA, and the plasmid origin of replication but the blaTEM-1 gene was replaced by the cat gene.

Using this engineering strategy a number of plasmids producing β-lactamase genes from different classes (see below) were generated using synthetic genes with an engineered NdeI restriction site at the 5′ end and BamHI restriction site at the 3′ end of each gene (GenScript). Both the synthetic β-lactamase genes and cat gene were ligated into the NdeI/BamHI sites of the pBR322 ΔNdeI PCR product and transformed into electrocompetent E. coli ElectroMax DH10B (Invitrogen/Life Technologies). E. coli DH10B harboring the recombinant plasmids were selected on LB agar (supplemented with 25 μg/mL tetracycline) and single isolated colonies were then inoculated into 5 mL LB media (supplemented with 25 μg/mL tetracycline), and incubated at 37° C. with aeration (250 rpm) for 18 hrs. The cultures were frozen back at −80° C. in 20% glycerol. The DNA sequence of the cloned β-lactamase genes was confirmed. The β-lactamase gene expression in the recombinant E. coli strains was driven by the blaTEM-1 promoter in the pBR-CBST plasmid and was characterized by MIC profiling of the E. coli recombinant strains against comparator β-lactam/BLI combinations in broth microdilution assay.

| | | | | GenBank |
|---|---|---|---|---|
| | | | Species | Accession |
| β-Lacta- | Name & | β- | Origin | Number of |
| mase | SEQ. ID of | Lacta- | of | β-Lacta- |
| Expressing | plasmids producing | mase | β-Lactamase | mase Gene |
| Strain | β-Lactamase | Class | Gene | Sequence |
| KPC-2 | pBR-CBST-KPC-2 | A | K. pneumoniae | EU784136 |
| | SEQID 6 | | | |
| CTX-M-15 | pBR-CBST-CTX-M-15 | A | K. pneumoniae | JF775516 |
| | SEQ ID 7 | | | |
| SHV-12 | pBR-CBST-SHV-12 | A | K. pneumoniae | AY008838 |
| | SEQ ID 8 | | | |
| P99 AmpC | pBR-CBST-P99 AMPC | C | E. cloacea | XO7274 |
| | SEQ ID 9 | | | |
| OXA-15 | pBR-CBST-OXA-15 | D | P. aeruginosa | PAU63835 |
| | SEQ ID 10 | | | |
| KPC-4 | pBR-CBST-KPC-4 | A | K. pneumoniae | EU447304 |
| | SEQ ID 11 | | | |
| DHA-1 | pBR-CBST-DHA-1 | C | K. pneumoniae | AY585202 |
| | SEQ ID 12 | | | |
| ADC-33 | pBR-CBST-ADC-33 | C | A. baumannii | EU687478 |
| | SEQ ID 13 | | | |

Nucleotide Sequences of pBR-CBST Plasmids (Containing β-Lactamase or cat Genes) Used in the E. coli Isogenic Strains (relevant restriction sites are underlined; β-lactamase sequences in all caps, tetA sequence is in italics)

pBR-CBST-cat
SEQ ID 5
ttcttgaagacgaaagggcctcgtgatacgcctatttttataggtta
atgtcatgataataatggtttcttagacgtcaggtggcacttttcgg
ggaaatgtgcgcggaacccctatttgtttatttttctaaatacattc
aaatatgtatccgctcatgagacaataaccctgataaatgcttcaat
aatattgaaaaaggaagagtcatATGGAGAAAAAAATCACTGGATAT
ACCACCGTTGATATATCCCAATGGCATCGTAAAGAACATTTTGAGGC
ATTTCAGTCAGTTGCTCAATGTACCTATAACCAGACCGTTCAGCTGG
ATATTACGGCCTTTTTAAAGACCGTAAAGAAAAATAAGCACAAGTTT
TATCCGGCCTTTATTCACATTCTTGCCCGCCTGATGAATGCTCATAC
GGAATTTCGTATGGCAATGAAAGACGGTGAGCTGGTGATATGGGATA
GTGTTCACCCTTGTTACACCGTTTTCCATGAGCAAACTGAAACGTTT
TCATCGCTCTGGAGTGAATACCACGACGATTTCCGGCAGTTTCTACA
CATATATTCGCAAGATGTGGCGTGTTACGGTGAAAACCTGGCCTATT
TCCCTAAAGGGTTTATTGAGAATATGTTTTTCGTCTCAGCCAATCCC
TGGGTGAGTTTCACCAGTTTTGATTTAAACGTGGCCAATATGGACAA
CTTCTTCGCCCCCGTTTTCACTATGGGCAAATATTATACGCAAGGCG
ACAAGGTGCTGATGCCGCTGGCGATTCAGGTTCATCATGCCGTCTGT
GATGGCTTCCATGTCGGCAGAATGCTTAATGAATTACAACAGTACTG
CGATGAGTGGCAGGGCGGGGCGTAAGTGGCAGGGCGGGGCGTAAGGC
GCGCCATTTAAATGAAGTTCCTATTCCGAAGTTCCTATTCTCTAGgg
atccctgtcagaccaagtttactcatatatactttagattgatttaa
aacttcatttttaatttaaaaggatctaggtgaagatcctttttgat
aatctcatgaccaaaatcccttaacgtgagttttcgttccactgagc
gtcagaccccgtagaaaagatcaaaggatcttcttgagatccttttt
ttctgcgcgtaatctgctgcttgcaaacaaaaaaaccaccgctacca
gcggtggtttgtttgccggatcaagagctaccaactctttttccgaa
ggtaactggcttcagcagagcgcagataccaaatactgtccttctag
tgtagccgtagttaggccaccacttcaagaactctgtagcaccgcct
acatacctcgctctgctaatcctgttaccagtggctgctgccagtgg
cgataagtcgtgtcttaccgggttggactcaagacgatagttaccgg
ataaggcgcagcggtcgggctgaacggggggttcgtgcacacagccc
agcttggagcgaacgacctacaccgaactgagatacctacagcgtga
gctatgagaaagcgccacgcttcccgaagggagaaaggcggacaggt
atccggtaagcggcagggtcggaacaggagagcgcacgagggagctt
ccagggggaaacgcctggtatctttatagtcctgtcgggtttcgcca
cctctgacttgagcgtcgatttttgtgatgctcgtcaggggggcgga
gcctatggaaaaacgccagcaacgcggcctttttacggttcctggcc
ttttgctggccttttgctcacatgttctttcctgcgttatcccctga
ttctgtggataaccgtattaccgcctttgagtgagctgataccgctc
gccgcagccgaacgaccgagcgcagcgagtcagtgagcgaggaagcg
gaagagcgcctgatgcggtattttctccttacgcatctgtgcggtat
ttcacaccgcatttggtgcactctcagtacaatctgctctgatgccg
catagttaagccagtatacactccgctatcgctacgtgactgggtca
tggctgcgccccgacacccgccaacacccgctgacgcgccctgacgg
gcttgtctgctcccggcatccgcttacagacaagctgtgaccgtctc
cgggagctgcatgtgtcagaggttttcaccgtcatcaccgaaacgcg
cgaggcagctgcggtaaagctcatcagcgtggtcgtgaagcgattca
cagatgtctgcctgttcatccgcgtccagctcgttgagtttctccag
aagcgttaatgtctggcttctgataaagcgggccatgttaagggcgg
ttttttcctgtttggtcactgatgcctccgtgtaagggggatttctg
ttcatgggggtaatgataccgatgaaacgagagaggatgctcacgat
acgggttactgatgatgaacatgcccggttactggaacgttgtgagg
gtaaacaactggcggtatggatgcggcgggaccagagaaaaatcact
cagggtcaatgccagcgcttcgttaatacagatgtaggtgttccaca
gggtagccagcagcatcctgcgatgcagatccggaacataatggtgc
agggcgctgacttccgcgtttccagactttacgaaacacggaaaccg
aagaccattcatgttgttgctcaggtcgcagacgttttgcagcagca
gtcgcttcacgttcgctcgcgtatcggtgattcattctgctaaccag
taaggcaaccccgccagcctagccgggtcctcaacgacaggagcacg
atcatgcgcacccgtggccaggacccaacgctgcccgagatgcgccg
cgtgcggctgctggagatggcggacgcgatggatatgttctgccaag
ggttggtttgcgcattcacagttctccgcaagaattgattggctcca
attcttggagtggtgaatccgttagcgaggtgccgccggcttccatt
caggtcgaggtggcccggctccatgcaccgcgacgcaacgcggggag
gcagacaaggtatagggcggcgcctacaatccatgccaacccgttcc
atgtgctcgccgaggcggcataaatcgccgtgacgatcagcggtcca
gtgatcgaagttaggctggtaagagccgcgagcgatccttgaagctg
tccctgatggtcgtcatctacctgcctggacagcatggcctgcaacg
cgggcatcccgatgccgccggaagcgagaagaatcataatggggaag
gccatccagcctcgcgtcgcgaacgccagcaagacgtagcccagcgc
gtcggccgccatgccggcgataatggcctgcttctcgccgaaacgtt
tggtggcgggaccagtgacgaaggcttgagcgagggcgtgcaagatt
ccgaataccgcaagcgacaggccgatcatcgtcgcgctccagcgaaa
gcggtcctcgccgaaaatgacccagagcgctgccggcacctgtccta
cgagttgcatgataaagaagacagtcataagtgcggcgacgatagtc
atgccccgcgcccaccggaaggagctgactgggttgaaggctctcaa
gggcatcggtcgacgctctcccttatgcgactcctgcattaggaagc
agcccagtagtaggttgaggccgttgagcaccgccgccgcaaggaat
ggtgcatgcaaggagatggcgcccaacagtcccccggccacggggcc
tgccaccatacccacgccgaaacaagcgctcatgagcccgaagtggc
gagcccgatcttccccatcggtgatgtcggcgatataggcgccagca
accgcacctgtggcgccggtgatgccggccacgatgcgtccggcgta
gaggattcacaggacgggtgtggtcgccatgatcgcgtagtcgatag
tggctccaagtagcgaagcgagcaggactgggcggcggccaaagcgg
tcggacagtgctccgagaacgggtgcgcatagaaattgcatcaacgc
atatagcgctagcagcacgccatagtgactggcgatgctgtcggaat
ggacgatatcccgcaagaggcccggcagtaccggcataaccaagcct
atgcctacagcatccagggtgacggtgccgaggatgacgatgagcgc
attgttagatttcatacacggtgcctgactgcgttagcaatttaact
gtgataaactaccgcattaaagcttatcgatgataagctgtcaaaca
tgagaa
pBR-CBST-KPC-2
SEQ ID 6
ttcttgaagacgaaagggcctcgtgatacgcctatttttataggtta
atgtcatgataataatggtttcttagacgtcaggtggcacttttcgg
ggaaatgtgcgcggaacccctatttgtttatttttctaaatacattc
aaatatgtatccgctcatgagacaataaccctgataaatgcttcaat
aatattgaaaaaggaagagtcatATGTCACTGTATCGCCGTCTAGTT
CTGCTGTCTTGTCTCTCATGGCCGCTGGCTGGCTTTTCTGCCACCGC
GCTGACCAACCTCGTCGCGGAACCATTCGCTAAACTCGAACAGGACT
TTGGCGGCTCCATCGGTGTGTACGCGATGGATACCGGCTCAGGCGCA
ACTGTAAGTTACCGCGCTGAGGAGCGCTTCCCACTGTGCAGCTCATT
CAAGGGCTTTCTTGCTGCCGCTGTGCTGGCTCGCAGCCAGCAGCAGG
CCGGCTTGCTGGACACACCCATCCGTTACGGCAAAAATGCGCTGGTT
CCGTGGTCACCCATCTCGGAAAAATATCTGACAACAGGCATGACGGT
GGCGGAGCTGTCCGCGGCCGCCGTGCAATACAGTGATAACGCCGCCG
CCAATTTGTTGCTGAAGGAGTTGGGCGGCCCGGCCGGGCTGACGGCC
TTCATGCGCTCTATCGGCGATACCACGTTCCGTCTGGACCGCTGGGA
GCTGGAGCTGAACTCCGCCATCCCAGGCGATGCGCGCGATACCTCAT
CGCCGCGCGCCGTGACGGAAAGCTTACAAAAACTGACACTGGGCTCT
GCACTGGCTGCGCCGCAGCGGCAGCAGTTTGTTGATTGGCTAAAGGG
AAACACGACCGGCAACCACCGCATCCGCGCGGCGGTGCCGGCAGACT
GGGCAGTCGGAGACAAAACCGGAACCTGCGGAGTGTATGGCACGGCA
AATGACTATGCCGTCGTCTGGCCCACTGGGCGCGCACCTATTGTGTT
GGCCGTCTACACCCGGGCGCCTAACAAGGATGACAAGCACAGCGAGG
CCGTCATCGCCGCTGCGGCTAGACTCGCGCTCGAGGGATTGGGCGTC
AACGGGCAGTAAggatccctgtcagaccaagtttactcatatatact
ttagattgatttaaaacttcatttttaatttaaaaggatctaggtga
agatcctttttgataatctcatgaccaaaatcccttaacgtgagttt
tcgttccactgagcgtcagaccccgtagaaaagatcaaaggatcttc
ttgagatcctttttttctgcgcgtaatctgctgcttgcaaacaaaaa
aaccaccgctaccagcggtggtttgtttgccggatcaagagctacca
actctttttccgaaggtaactggcttcagcagagcgcagataccaaa
tactgtccttctagtgtagccgtagttaggccaccacttcaagaact
ctgtagcaccgcctacatacctcgctctgctaatcctgttaccagtg
gctgctgccagtggcgataagtcgtgtcttaccgggttggactcaag
acgatagttaccggataaggcgcagcggtcgggctgaacggggggtt
cgtgcacacagcccagcttggagcgaacgacctacaccgaactgaga
tacctacagcgtgagctatgagaaagcgccacgcttcccgaagggag
aaaggcggacaggtatccggtaagcggcagggtcggaacaggagagc
gcacgagggagcttccagggggaaacgcctggtatctttatagtcct
gtcgggtttcgccacctctgacttgagcgtcgatttttgtgatgctc
gtcaggggggcggagcctatggaaaaacgccagcaacgcggcctttt
tacggttcctggccttttgctggccttttgctcacatgttctttcct
gcgttatcccctgattctgtggataaccgtattaccgcctttgagtg
agctgataccgctcgccgcagccgaacgaccgagcgcagcgagtcag
tgagcgaggaagcggaagagcgcctgatgcggtattttctccttacg
catctgtgcggtatttcacaccgcatttggtgcactctcagtacaat
ctgctctgatgccgcatagttaagccagtatacactccgctatcgct
acgtgactgggtcatggctgcgccccgacacccgccaacacccgctg
acgcgccctgacgggcttgtctgctcccggcatccgcttacagacaa
gctgtgaccgtctccgggagctgcatgtgtcagaggttttcaccgtc
atcaccgaaacgcgcgaggcagctgcggtaaagctcatcagcgtggt
cgtgaagcgattcacagatgtctgcctgttcatccgcgtccagctcg
ttgagtttctccagaagcgttaatgtctggcttctgataaagcgggc
catgttaagggcggttttttcctgtttggtcactgatgcctccgtgt
aagggggatttctgttcatgggggtaatgataccgatgaaacgagag
aggatgctcacgatacgggttactgatgatgaacatgcccggttact
ggaacgttgtgagggtaaacaactggcggtatggatgcggcgggacc
agagaaaaatcactcagggtcaatgccagcgcttcgttaatacagat
gtaggtgttccacagggtagccagcagcatcctgcgatgcagatccg
gaacataatggtgcagggcgctgacttccgcgtttccagactttacg
aaacacggaaaccgaagaccattcatgttgttgctcaggtcgcagac
gttttgcagcagcagtcgcttcacgttcgctcgcgtatcggtgattc
attctgctaaccagtaaggcaaccccgccagcctagccgggtcctca
acgacaggagcacgatcatgcgcacccgtggccaggacccaacgctg
cccgagatgcgccgcgtgcggctgctggagatggcggacgcgatgga
tatgttctgccaagggttggtttgcgcattcacagttctccgcaaga
attgattggctccaattcttggagtggtgaatccgttagcgaggtgc
cgccggcttccattcaggtcgaggtggcccggctccatgcaccgcga
cgcaacgcggggaggcagacaaggtatagggcggcgcctacaatcca
tgccaacccgttccatgtgctcgccgaggcggcataaatcgccgtga
cgatcagcggtccagtgatcgaagttaggctggtaagagccgcgagc
gatccttgaagctgtccctgatggtcgtcatctacctgcctggacag
catggcctgcaacgcgggcatcccgatgccgccggaagcgagaagaa
tcataatggggaaggccatccagcctcgcgtcgcgaacgccagcaag
acgtagcccagcgcgtcggccgccatgccggcgataatggcctgctt
ctcgccgaaacgtttggtggcgggaccagtgacgaaggcttgagcga
gggcgtgcaagattccgaataccgcaagcgacaggccgatcatcgtc
gcgctccagcgaaagcggtcctcgccgaaaatgacccagagcgctgc
cggcacctgtcctacgagttgcatgataaagaagacagtcataagtg
cggcgacgatagtcatgccccgcgcccaccggaaggagctgactggg
ttgaaggctctcaagggcatcggtcgacgctctcccttatgcgactc
ctgcattaggaagcagcccagtagtaggttgaggccgttgagcaccg
ccgccgcaaggaatggtgcatgcaaggagatggcgcccaacagtccc
ccggccacggggcctgccaccatacccacgccgaaacaagcgctcat
gagcccgaagtggcgagcccgatcttccccatcggtgatgtcggcga
tataggcgccagcaaccgcacctgtggcgccggtgatgccggccacg
atgcgtccggcgtagaggattcacaggacgggtgtggtcgccatgat
cgcgtagtcgatagtggctccaagtagcgaagcgagcaggactgggc
ggcggccaaagcggtcggacagtgctccgagaacgggtgcgcataga
aattgcatcaacgcatatagcgctagcagcacgccatagtgactggc
gatgctgtcggaatggacgatatcccgcaagaggcccggcagtaccg
gcataaccaagcctatgcctacagcatccagggtgacggtgccgagg
atgacgatgagcgcattgttagatttcatacacggtgcctgactgcg
ttagcaatttaactgtgataaactaccgcattaaagcttatcgatga
taagctgtcaaacatgagaa
pBR-CBST-CTX-M-15
SEQ ID 7
ttcttgaagacgaaagggcctcgtgatacgcctatttttataggtta
atgtcatgataataatggtttcttagacgtcaggtggcacttttcgg
ggaaatgtgcgcggaacccctatttgtttatttttctaaatacattc
aaatatgtatccgctcatgagacaataaccctgataaatgcttcaat
aatattgaaaaaggaagagtcatATGGAATCTGTTAAATCAGCGAGT
TGAGATCAAAAAATCTGACCTTGTTAACTATAATCCGATTGCGGAAA
AGCACGTCAATGGGACGATGTCACTGGCTGAGCTTAGCGCGGCCGCG
CTACAGTACAGCGATAACGTGGCGATGAATAAGCTGATTGCTCACGT
TGGCGGCCCGGCTAGCGTCACCGCGTTCGCCCGACAGCTGGGAGACG
AAACGTTCCGTCTCGACCGTACCGAGCCGACGTTAAACACCGCCATT
CCGGGCGATCCGCGTGATACCACTTCACCTCGGGCAATGGCGCAAAC
TCTGCGGAATCTGACGCTGGGTAAAGCATTGGGCGACAGCCAACGGG
CGCAGCTGGTGACATGGATGAAAGGCAATACCACCGGTGCAGCGAGC
ATTCAGGCTGGACTGCCTGCTTCCTGGGTTGTGGGGGATAAAACCGG
CAGCGGTGGCTATGGCACCACCAACGATATCGCGGTGATCTGGCCAA
AAGATCGTGCGCCGCTGATTCTGGTCACTTACTTCACCCAGCCTCAA
CCTAAGGCAGAAAGCCGTCGCGATGTATTAGCGTCGGCGGCTAAAAT
CGTCACCGACGGTTTGTAAggatccctgtcagaccaagtttactcat
atatactttagattgatttaaaacttcatttttaatttaaaaggatc
taggtgaagatcctttttgataatctcatgaccaaaatcccttaacg
tgagttttcgttccactgagcgtcagaccccgtagaaaagatcaaag
gatcttcttgagatcctttttttctgcgcgtaatctgctgcttgcaa
acaaaaaaaccaccgctaccagcggtggtttgtttgccggatcaaga
gctaccaactctttttccgaaggtaactggcttcagcagagcgcaga
taccaaatactgtccttctagtgtagccgtagttaggccaccacttc
aagaactctgtagcaccgcctacatacctcgctctgctaatcctgtt
accagtggctgctgccagtggcgataagtcgtgtcttaccgggttgg
actcaagacgatagttaccggataaggcgcagcggtcgggctgaacg
gggggttcgtgcacacagcccagcttggagcgaacgacctacaccga
actgagatacctacagcgtgagctatgagaaagcgccacgcttcccg
aagggagaaaggcggacaggtatccggtaagcggcagggtcggaaca
ggagagcgcacgagggagcttccagggggaaacgcctggtatcttta
tagtcctgtcgggtttcgccacctctgacttgagcgtcgatttttgt
gatgctcgtcaggggggcggagcctatggaaaaacgccagcaacgcg
gcctttttacggttcctggccttttgctggccttttgctcacatgtt
ctttcctgcgttatcccctgattctgtggataaccgtattaccgcct
ttgagtgagctgataccgctcgccgcagccgaacgaccgagcgcagc
gagtcagtgagcgaggaagcggaagagcgcctgatgcggtattttct
ccttacgcatctgtgcggtatttcacaccgcatttggtgcactctca
gtacaatctgctctgatgccgcatagttaagccagtatacactccgc
tatcgctacgtgactgggtcatggctgcgccccgacacccgccaaca
cccgctgacgcgccctgacgggcttgtctgctcccggcatccgctta
cagacaagctgtgaccgtctccgggagctgcatgtgtcagaggtttt
caccgtcatcaccgaaacgcgcgaggcagctgcggtaaagctcatca
gcgtggtcgtgaagcgattcacagatgtctgcctgttcatccgcgtc
cagctcgttgagtttctccagaagcgttaatgtctggcttctgataa
agcgggccatgttaagggcggttttttcctgtttggtcactgatgcc
tccgtgtaagggggatttctgttcatgggggtaatgataccgatgaa
acgagagaggatgctcacgatacgggttactgatgatgaacatgccc
ggttactggaacgttgtgagggtaaacaactggcggtatggatgcgg
cgggaccagagaaaaatcactcagggtcaatgccagcgcttcgttaa
tacagatgtaggtgttccacagggtagccagcagcatcctgcgatgc
agatccggaacataatggtgcagggcgctgacttccgcgtttccaga
ctttacgaaacacggaaaccgaagaccattcatgttgttgctcaggt
cgcagacgttttgcagcagcagtcgcttcacgttcgctcgcgtatcg
gtgattcattctgctaaccagtaaggcaaccccgccagcctagccgg
gtcctcaacgacaggagcacgatcatgcgcacccgtggccaggaccc
aacgctgcccgagatgcgccgcgtgcggctgctggagatggcggacg
cgatggatatgttctgccaagggttggtttgcgcattcacagttctc
cgcaagaattgattggctccaattcttggagtggtgaatccgttagc
gaggtgccggcttccattcaggtcgaggtggcccggctccatgcacc
gcgacgcaacgcggggaggcagacaaggtatagggcggcgcctacaa
tccatgccaacccgttccatgtgctcgccgaggcggcataaatcgcc
gtgacgatcagcggtccagtgatcgaagttaggctggtaagagccgc
gagcgatccttgaagctgtccctgatggtcgtcatctacctgcctgg
acagcatggcctgcaacgcgggcatcccgatgccgccggaagcgaga
agaatcataatggggaaggccatccagcctcgcgtcgcgaacgccag
caagacgtagcccagcgcgtcggccgccatgccggcgataatggcct
gcttctcgccgaaacgtttggtggcgggaccagtgacgaaggcttga
gcgagggcgtgcaagattccgaataccgcaagcgacaggccgatcat
cgtcgcgctccagcgaaagcggtcctcgccgaaaatgacccagagcg
ctgccggcacctgtcctacgagttgcatgataaagaagacagtcata
agtgcggcgacgatagtcatgccccgcgcccaccggaaggagctgac
tgggugaaggctctcaagggcatcggtcgacgctctcccttatgcga
ctcctgcattaggaagcagcccagtagtaggttgaggccgttgagca
ccgccgccgcaaggaatggtgcatgcaaggagatggcgcccaacagt
cccccggccacggggcctgccaccatacccacgccgaaacaagcgct
catgagcccgaagtggcgagcccgatcttccccatcggtgatgtcgg
cgatataggcgccagcaaccgcacctgtggcgccggtgatgccggcc
acgatgcgtccggcgtagaggattcacaggacgggtgtggtcgccat
gatcgcgtagtcgatagtggctccaagtagcgaagcgagcaggactg
ggcggcggccaaagcggtcggacagtgctccgagaacgggtgcgcat
agaaattgcatcaacgcatatagcgctagcagcacgccatagtgact
ggcgatgctgtcggaatggacgatatcccgcaagaggcccggcagta
ccggcataaccaagcctatgcctacagcatccagggtgacggtgccg
aggatgacgatgagcgcattgttagatttcatacacggtgcctgact
gcgttagcaatttaactgtgataaactaccgcattaaagcttatcga
tgataagctgtcaaacatgagaa
pBR-CBST-SHV-12
SEQ ID 8
ttcttgaagacgaaagggcctcgtgatacgcctatttttataggtta
atgtcatgataataatggtttcttagacgtcaggtggcacttttcgg
ggaaatgtgcgcggaacccctatttgtttatttttctaaatacattc
aaatatgtatccgctcatgagacaataaccctgataaatgcttcaat
aatattgaaaaaggaagagtcatATGCGTTATATTCGCCTGTGTATT
ATCTCCCTGTTAGCCACCCTGCCGCTGGCGGTACACGCCAGCCCGCA
GCCGCTTGAGCAAATTAAACAAAGCGAAAGCCAGCTGTCGGGCCGCG
TAGGCATGATAGAAATGGATCTGGCCAGCGGCCGCACGCTGACCGCC
TGGCGCGCCGATGAACGCTTTCCCATGATGAGCACCTTTAAAGTAGT
GCTCTGCGGCGCAGTGCTGGCGCGGGTGGATGCCGGTGACGAACAGC
TGGAGCGAAAGATCCACTATCGCCAGCAGGATCTGGTGGACTACTCG
CCGGTCAGCGAAAAACACCTTGCCGACGGCATGACGGTCGGCGAACT
CTGCGCCGCCGCCATTACCATGAGCGATAACAGCGCCGCCAATCTGC
TGCTGGCCACCGTCGGCGGCCCCGCAGGATTGACTGCCTTTTTGCGC
CAGATCGGCGACAACGTCACCCGCCTTGACCGCTGGGAAACGGAACT
GAATGAGGCGCTTCCCGGCGACGCCCGCGACACCACTACCCCGGCCA
GCATGGCCGCGACCCTGCGCAAGCTGCTGACCAGCCAGCGTCTGAGC
GCCCGTTCGCAACGGCAGCTGCTGCAGTGGATGGTGGACGATCGGGT
CGCCGGACCGTTGATCCGCTCCGTGCTGCCGGCGGGCTGGTTTATCG
CCGATAAGACCGGAGCTAGCAAGCGGGGTGCGCGCGGGATTGTCGCC
CTGCTTGGCCCGAATAACAAAGCAGAGCGCATTGTGGTGATTTATCT
GCGGGATACCCCGGCGAGCATGGCCGAGCGAAATCAGCAAATCGCCG
GGATCGGCGCGGCGCTGATCGAGCACTGGCAACGCTAAggatccctg
tcagaccaagtttactcatatatactttagattgatttaaaacttca
tttttaatttaaaaggatctaggtgaagatcctttttgataatctca
tgaccaaaatcccttaacgtgagttttcgttccactgagcgtcagac
cccgtagaaaagatcaaaggatcttcttgagatcctttttttctgcg
cgtaatctgctgcttgcaaacaaaaaaaccaccgctaccagcggtgg
tttgtttgccggatcaagagctaccaactctttttccgaaggtaact
ggcttcagcagagcgcagataccaaatactgtccttctagtgtagcc
gtagttaggccaccacttcaagaactctgtagcaccgcctacatacc
tcgctctgctaatcctgttaccagtggctgctgccagtggcgataag
tcgtgtcttaccgggttggactcaagacgatagttaccggataaggc
gcagcggtcgggctgaacggggggttcgtgcacacagcccagcttgg
agcgaacgacctacaccgaactgagatacctacagcgtgagctatga
gaaagcgccacgcttcccgaagggagaaaggcggacaggtatccggt
aagcggcagggtcggaacaggagagcgcacgagggagcttccagggg
gaaacgcctggtatctttatagtcctgtcgggtttcgccacctctga
cttgagcgtcgatttttgtgatgctcgtcaggggggcggagcctatg
gaaaaacgccagcaacgcggcctttttacggttcctggccttttgct
ggccttttgctcacatgttctttcctgcgttatcccctgattctgtg
gataaccgtattaccgcctttgagtgagctgataccgctcgccgcag
ccgaacgaccgagcgcagcgagtcagtgagcgaggaagcggaagagc
gcctgatgcggtattttctccttacgcatctgtgcggtatttcacac
cgcatttggtgcactctcagtacaatctgctctgatgccgcatagtt
aagccagtatacactccgctatcgctacgtgactgggtcatggctgc
gccccgacacccgccaacacccgctgacgcgccctgacgggcttgtc
tgctcccggcatccgcttacagacaagctgtgaccgtctccgggagc
tgcatgtgtcagaggttttcaccgtcatcaccgaaacgcgcgaggca
gctgcggtaaagctcatcagcgtggtcgtgaagcgattcacagatgt
ctgcctgttcatccgcgtccagctcgttgagtttctccagaagcgtt
aatgtctggcttctgataaagcgggccatgttaagggcggttttttc
ctgtttggtcactgatgcctccgtgtaagggggatttctgttcatgg
gggtaatgataccgatgaaacgagagaggatgctcacgatacgggtt
actgatgatgaacatgcccggttactggaacgttgtgagggtaaaca
actggcggtatggatgcggcgggaccagagaaaaatcactcagggtc
aatgccagcgcttcgttaatacagatgtaggtgttccacagggtagc
cagcagcatcctgcgatgcagatccggaacataatggtgcagggcgc
tgacttccgcgtttccagactttacgaaacacggaaaccgaagacca
ttcatgttgttgctcaggtcgcagacgttttgcagcagcagtcgctt
cacgttcgctcgcgtatcggtgattcattctgctaaccagtaaggca
accccgccagcctagccgggtcctcaacgacaggagcacgatcatgc
gcacccgtggccaggacccaacgctgcccgagatgcgccgcgtgcgg
ctgctggagatggcggacgcgatggatatgttctgccaagggttggt
ttgcgcattcacagttctccgcaagaattgattggctccaattcttg
gagtggtgaatccgttagcgaggtgccgccggcttccattcaggtcg
aggtggcccggctccatgcaccgcgacgcaacgcggggaggcagaca
aggtatagggcggcgcctacaatccatgccaacccgttccatgtgct
cgccgaggcggcataaatcgccgtgacgatcagcggtccagtgatcg
aagttaggctggtaagagccgcgagcgatccttgaagctgtccctga
tggtcgtcatctacctgcctggacagcatggcctgcaacgcgggcat
cccgatgccgccggaagcgagaagaatcataatggggaaggccatcc
agcctcgcgtcgcgaacgccagcaagacgtagcccagcgcgtcggcc
gccatgccggcgataatggcctgcttctcgccgaaacgtttggtggc
gggaccagtgacgaaggcttgagcgagggcgtgcaagattccgaata
ccgcaagcgacaggccgatcatcgtcgcgctccagcgaaagcggtcc
tcgccgaaaatgacccagagcgctgccggcacctgtcctacgagttg
catgataaagaagacagtcataagtgcggcgacgatagtcatgcccc
gcgcccaccggaaggagctgactgggttgaaggctctcaagggcatc
ggtcgacgctctcccttatgcgactcctgcattaggaagcagcccag
tagtaggttgaggccgttgagcaccgccgccgcaaggaatggtgcat
gcaaggagatggcgcccaacagtcccccggccacggggcctgccacc
atacccacgccgaaacaagcgctcatgagcccgaagtggcgagcccg
atcttccccatcggtgatgtcggcgatataggcgccagcaaccgcac
ctgtggcgccggtgatgccggccacgatgcgtccggcgtagaggatt
cacaggacgggtgtggtcgccatgatcgcgtagtcgatagtggctcc
aagtagcgaagcgagcaggactgggcggcggccaaagcggtcggaca
gtgctccgagaacgggtgcgcatagaaattgcatcaacgcatatagc
gctagcagcacgccatagtgactggcgatgctgtcggaatggacgat
atcccgcaagaggcccggcagtaccggcataaccaagcctatgccta
cagcatccagggtgacggtgccgaggatgacgatgagcgcattgtta
gatttcatacacggtgcctgactgcgttagcaatttaactgtgataa
actaccgcattaaagcttatcgatgataagctgtcaaacatgagaa
pBR-CBST-P99
SEQ ID 9
ttcttgaagacgaaagggcctcgtgatacgcctatttttataggtta
atgtcatgataataatggtttcttagacgtcaggtggcacttttcgg
ggaaatgtgcgcggaacccctatttgtttatttttctaaatacattc
aaatatgtatccgctcatgagacaataaccctgataaatgcttcaat
aatattgaaaaaggaagagtcatATGATGAGAAAATCCCTTTGCTGC
GCCCTGCTGCTCGGCATCTCTTGCTCTGCTCTCGCCACGCCAGTGTC
AGAAAAACAGCTGGCGGAGGTGGTCGCGAATACGATTACCCCGCTGA
TGAAAGCCCAGTCTGTTCCAGGCATGGCGGTGGCCGTTATTTATCAG
GGAAAACCGCACTATTACACATTTGGCAAGGCCGATATCGCGGCGAA
TAAACCCGTTACGCCTCAGACCCTGTTCGAGCTGGGTTCTATAAGTA
AAACCTTCACCGGCGTTTTAGGTGGGGATGCCATTGCTCGCGGTGAA
ATTTCGCTGGACGATGCGGTGACCAGATACTGGCCACAGCTGACGGG
CAAGCAGTGGCAGGGTATTCGTATGCTGGATCTCGCCACCTACACCG
CTGGCGGCCTGCCGCTACAGGTACCGGATGAGGTCACGGATAACGCC
TCCCTGCTGCGCTTTTATCAAAACTGGCAGCCGCAGTGGAAGCCTGG
CACAACGCGTCTTTACGCCAACGCCAGCATCGGTCTTTTTGGTGCGC
TGGCGGTCAAACCTTCTGGCATGCCCTATGAGCAGGCCATGACGACG
CGGGTCCTTAAGCCGCTCAAGCTGGACCATACCTGGATTAACGTGCC
GAAAGCGGAAGAGGCGCATTACGCCTGGGGCTATCGTGACGGTAAAG
CGGTGCGCGTTTCGCCGGGTATGCTGGATGCACAAGCCTATGGCGTG
AAAACCAACGTGCAGGATATGGCGAACTGGGTCATGGCAAACATGGC
GCCGGAGAACGTTGCTGATGCCTCACTTAAGCAGGGCATCGCGCTGG
CGCAGTCGCGCTACTGGCGTATCGGGTCAATGTATCAGGGTCTGGGC
TGGGAGATGCTCAACTGGCCCGTGGAGGCCAACACGGTGGTCGAGGG
CAGCGACAGTAAGGTAGCACTGGCGCCGTTGCCCGTGGCAGAAGTGA
ATCCACCGGCTCCCCCGGTCAAAGCGTCCTGGGTCCATAAAACGGGC
TCTACTGGCGGGTTTGGCAGCTACGTGGCCTTTATTCCTGAAAAGCA
GATCGGTATTGTGATGCTCGCGAATACAAGCTATCCGAACCCGGCAC
GCGTTGAGGCGGCATACCATATCCTCGAGGCGCTACAGTAAggatcc
ctgtcagaccaagtttactcatatatactttagattgatttaaaact
tcatttttaatttaaaaggatctaggtgaagatcctttttgataatc
tcatgaccaaaatcccttaacgtgagttttcgttccactgagcgtca
gaccccgtagaaaagatcaaaggatcttcttgagatcctttttttct
gcgcgtaatctgctgcttgcaaacaaaaaaaccaccgctaccagcgg
tggtttgtttgccggatcaagagctaccaactctttttccgaaggta
actggcttcagcagagcgcagataccaaatactgtecttctagtgta
gccgtagttaggccaccacttcaagaactctgtagcaccgcctacat
acctcgctctgctaatcctgttaccagtggctgctgccagtggcgat
aagtcgtgtcttaccgggttggactcaagacgatagttaccggataa
ggcgcagcggtcgggctgaacggggggttcgtgcacacagcccagct
tggagcgaacgacctacaccgaactgagatacctacagcgtgagcta
tgagaaagcgccacgcttcccgaagggagaaaggcggacaggtatcc
ggtaagcggcagggtcggaacaggagagcgcacgagggagcttccag
ggggaaacgcctggtatctttatagtcctgtcgggtttcgccacctc
tgacttgagcgtcgatttttgtgatgctcgtcaggggggcggagcct
atggaaaaacgccagcaacgcggcctttttacggttcctggcctttt
gctggccttttgctcacatgttctttcctgcgttatcccctgattct
gtggataaccgtattaccgcctttgagtgagctgataccgctcgccg
cagccgaacgaccgagcgcagcgagtcagtgagcgaggaagcggaag
agcgcctgatgcggtattttctccttacgcatctgtgcggtatttca
caccgcatttggtgcactctcagtacaatctgctctgatgccgcata
gttaagccagtatacactccgctatcgctacgtgactgggtcatggc
tgcgccccgacacccgccaacacccgctgacgcgccctgacgggctt
gtctgctcccggcatccgcttacagacaagctgtgaccgtctccggg
agctgcatgtgtcagaggttttcaccgtcatcaccgaaacgcgcgag
gcagctgcggtaaagctcatcagcgtggtcgtgaagcgattcacaga
tgtctgcctgttcatccgcgtccagctcgttgagtttctccagaagc
gttaatgtctggcttctgataaagcgggccatgttaagggcggtttt
ttcctgtttggtcactgatgcctccgtgtaagggggatttctgttca
tgggggtaatgataccgatgaaacgagagaggatgctcacgatacgg
gttactgatgatgaacatgcccggttactggaacgttgtgagggtaa
acaactggcggtatggatgcggcgggaccagagaaaaatcactcagg
gtcaatgccagcgcttcgttaatacagatgtaggtgttccacagggt
agccagcagcatcctgcgatgcagatccggaacataatggtgcaggg
cgctgacttccgcgtttccagactttacgaaacacggaaaccgaaga
ccattcatgttgttgctcaggtcgcagacgttttgcagcagcagtcg
cttcacgttcgctcgcgtatcggtgattcattctgctaaccagtaag
gcaaccccgccagcctagccgggtcctcaacgacaggagcacgatca
tgcgcacccgtggccaggacccaacgctgcccgagatgcgccgcgtg
cggctgctggagatggcggacgcgatggatatgttctgccaagggtt
ggtttgcgcattcacagttctccgcaagaattgattggctccaattc
ttggagtggtgaatccgttagcgaggtgccgccggcttccattcagg
tcgaggtggcccggctccatgcaccgcgacgcaacgcggggaggcag
acaaggtatagggcggcgcctacaatccatgccaacccgttccatgt
gctcgccgaggcggcataaatcgccgtgacgatcagcggtccagtga
tcgaagttaggctggtaagagccgcgagcgatccttgaagctgtccc
tgatggtcgtcatctacctgcctggacagcatggcctgcaacgcggg
catcccgatgccgccggaagcgagaagaatcataatggggaaggcca
tccagcctcgcgtcgcgaacgccagcaagacgtagcccagcgcgtcg
gccgccatgccggcgataatggcctgcttctcgccgaaacgtttggt
ggcgggaccagtgacgaaggcttgagcgagggcgtgcaagattccga
ataccgcaagcgacaggccgatcatcgtcgcgctccagcgaaagcgg
tcctcgccgaaaatgacccagagcgctgccggcacctgtcctacgag
ttgcatgataaagaagacagtcataagtgcggcgacgatagtcatgc
cccgcgcccaccggaaggagctgactgggttgaaggctctcaagggc
atcggtcgacgctctcccttatgcgactcctgcattaggaagcagcc
cagtagtaggttgaggccgttgagcaccgccgccgcaaggaatggtg
catgcaaggagatggcgcccaacagtcccccggccacggggcctgcc
accatacccacgccgaaacaagcgctcatgagcccgaagtggcgagc
ccgatcttccccatcggtgatgtcggcgatataggcgccagcaaccg
cacctgtggcgccggtgatgccggccacgatgcgtccggcgtagagg
attcacaggacgggtgtggtcgccatgatcgcgtagtcgatagtggc
tccaagtagcgaagcgagcaggactgggcggcggccaaagcggtcgg
acagtgctccgagaacgggtgcgcatagaaattgcatcaacgcatat
agcgctagcagcacgccatagtgactggcgatgctgtcggaatggac
gatatcccgcaagaggcccggcagtaccggcataaccaagcctatgc
ctacagcatccagggtgacggtgccgaggatgacgatgagcgcattg
ttagatttcatacacggtgcctgactgcgttagcaatttaactgtga
taaactaccgcattaaagcttatcgatgataagctgtcaaacatgag
aa
pBR-CBST-OXA-15
SEQ ID 10
ttcttgaagacgaaagggcctcgtgatacgcctatttttataggtta
atgtcatgataataatggtttcttagacgtcaggtggcacttttcgg
ggaaatgtgcgcggaacccctatttgtttatttttctaaatacattc
aaatatgtatccgctcatgagacaataaccctgataaatgcttcaat
aatattgaaaaaggaagagtcatATGGCAATCCGAATCTTCGCGATA
CTTTTCTCCATTTTTTCTCTTGCCACTTTCGCGCATGCGCAAGAAGG
CACGCTAGAACGTTCTGACTGGAGGAAGTTTTTCAGCGAATTTCAAG
CCAAAGGCACGATAGTTGTGGCAGACGAACGCCAAGCGGATCGTGCC
ATGTTGGTTTTTGATCCTGTGCGATCGAAGAAACGCTACTCGCCTGC
ATCGACATTCAAGATACCTCATACACTTTTTGCACTTGATGCAGGCG
CTGTTCGTGATGAGTTCCAGATTTTTCGATGGGACGGCGTTAACAGG
GGCTTTGCAGGCCACAATCAAGACCAAGATTTGCGATCAGCAATGCG
GAATTCTACTGTTTGGGTGTATGAGCTATTTGCAAAGGAAATTGGTG
ATGACAAAGCTCGGCGCTATTTGAAGAAAATCGACTATGGCAACGCC
GGTCCTTCGACAAGTAATGGCGATTACTGGATAGAAGGCAGCCTTGC
AATCTCGGCGCAGGAGCAAATTGCATTTCTCAGGAAGCTCTATCGTA
ACGAGCTGCCCTTTCGGGTAGAACATCAGCGCTTGGTCAAGGATCTC
ATGATTGTGGAAGCCGGTCGCAACTGGATACTGCGTGCAAAGACGGG
CTGGGAAGGCCGTATGGGTTGGTGGGTAGGATGGGTTGAGTGGCCGA
CTGGCTCCGTATTCTTCGCACTGAATATTGATACGCCAAACAGAATG
GATGATCTTTTCAAGAGGGAGGCAATCGTGCGGGCAATCCTTCGCTC
TATTGAAGCGTTACCGCCCAACCCGGCAGTCAACTCGGACGCTGCGC
GATAAggatccctgtcagaccaagtttactcatatatactttagatt
gatttaaaacttcatttttaatttaaaaggatctaggtgaagatcct
ttttgataatctcatgaccaaaatcccttaacgtgagttttcgttcc
actgagcgtcagaccccgtagaaaagatcaaaggatcttcttgagat
cctttttttctgcgcgtaatctgctgcttgcaaacaaaaaaaccacc
gctaccagcggtggtttgtttgccggatcaagagctaccaactcttt
ttccgaaggtaactggcttcagcagagcgcagataccaaatactgtc
cttctagtgtagccgtagttaggccaccacttcaagaactctgtagc
accgcctacatacctcgctctgctaatcctgttaccagtggctgctg
ccagtggcgataagtcgtgtcttaccgggttggactcaagacgatag
ttaccggataaggcgcagcggtcgggctgaacggggggttcgtgcac
acagcccagcttggagcgaacgacctacaccgaactgagatacctac
agcgtgagctatgagaaagcgccacgcttcccgaagggagaaaggcg
gacaggtatccggtaagcggcagggtcggaacaggagagcgcacgag
ggagcttccagggggaaacgcctggtatctttatagtcctgtcgggt
ttcgccacctctgacttgagcgtcgatttttgtgatgctcgtcaggg
gggcggagcctatggaaaaacgccagcaacgcggcctttttacggtt
cctggccttttgctggccttttgctcacatgttctttcctgcgttat
cccctgattctgtggataaccgtattaccgcctttgagtgagctgat
accgctcgccgcagccgaacgaccgagcgcagcgagtcagtgagcga
ggaagcggaagagcgcctgatgcggtattttctccttacgcatctgt
gcggtatttcacaccgcatttggtgcactctcagtacaatctgctct
gatgccgcatagttaagccagtatacactccgctatcgctacgtgac
tgggtcatggctgcgccccgacacccgccaacacccgctgacgcgcc
ctgacgggcttgtctgctcccggcatccgcttacagacaagctgtga
ccgtctccgggagctgcatgtgtcagaggttttcaccgtcatcaccg
aaacgcgcgaggcagctgcggtaaagctcatcagcgtggtcgtgaag
cgattcacagatgtctgcctgttcatccgcgtccagctcgttgagtt
tctccagaagcgttaatgtctggcttctgataaagcgggccatgtta
agggcggttttttcctgtttggtcactgatgcctccgtgtaaggggg
atttctgttcatgggggtaatgataccgatgaaacgagagaggatgc
tcacgatacgggttactgatgatgaacatgcccggttactggaacgt
tgtgagggtaaacaactggcggtatggatgcggcgggaccagagaaa
aatcactcagggtcaatgccagcgcttcgttaatacagatgtaggtg
ttccacagggtagccagcagcatcctgcgatgcagatccggaacata
atggtgcagggcgctgacttccgcgtttccagactttacgaaacacg
gaaaccgaagaccattcatgttgttgctcaggtcgcagacgttttgc
agcagcagtcgcttcacgttcgctcgcgtatcggtgattcattctgc
taaccagtaaggcaaccccgccagcctagccgggtcctcaacgacag
gagcacgatcatgcgcacccgtggccaggacccaacgctgcccgaga
tgcgccgcgtgcggctgctggagatggcggacgcgatggatatgttc
tgccaagggttggtttgcgcattcacagttctccgcaagaattgatt
ggctccaattcttggagtggtgaatccgttagcgaggtgccgccggc
ttccattcaggtcgaggtggcccggctccatgcaccgcgacgcaacg
cggggaggcagacaaggtatagggcggcgcctacaatccatgccaac
ccgttccatgtgctcgccgaggcggcataaatcgccgtgacgatcag
cggtccagtgatcgaagttaggctggtaagagccgcgagcgatcctt
gaagctgtccctgatggtcgtcatctacctgcctggacagcatggcc
tgcaacgcgggcatcccgatgccgccggaagcgagaagaatcataat
ggggaaggccatccagcctcgcgtcgcgaacgccagcaagacgtagc
ccagcgcgtcggccgccatgccggcgataatggcctgcttctcgccg
aaacgtttggtggcgggaccagtgacgaaggcttgagcgagggcgtg
caagattccgaataccgcaagcgacaggccgatcatcgtcgcgctcc
agcgaaagcggtcctcgccgaaaatgacccagagcgctgccggcacc
tgtcctacgagttgcatgataaagaagacagtcataagtgcggcgac
gatagtcatgccccgcgcccaccggaaggagctgactgggttgaagg
ctctcaagggcatcggtcgacgctctcccttatgcgactcctgcatt
aggaagcagcccagtagtaggttgaggccgttgagcaccgccgccgc
aaggaatggtgcatgcaaggagatggcgcccaacagtcccccggcca
cggggcctgccaccatacccacgccgaaacaagcgctcatgagcccg
aagtggcgagcccgatcttccccatcggtgatgtcggcgatataggc
gccagcaaccgcacctgtggcgccggtgatgccggccacgatgcgtc
cggcgtagaggattcacaggacgggtgtggtcgccatgatcgcgtag
tcgatagtggctccaagtagcgaagcgagcaggactgggcggcggcc
aaagcggtcggacagtgctccgagaacgggtgcgcatagaaattgca
tcaacgcatatagcgctagcagcacgccatagtgactggcgatgctg
tcggaatggacgatatcccgcaagaggcccggcagtaccggcataac
caagcctatgcctacagcatccagggtgacggtgccgaggatgacga
tgagcgcattgttagatttcatacacggtgcctgactgcgttagcaa
tttaactgtgataaactaccgcattaaagcttatcgatgataagctg
tcaaacatgagaa
pBR-CBST-KPC-4
SEQ ID 11
ttcttgaagacgaaagggcctcgtgatacgcctatttttataggtta
atgtcatgataataatggtttcttagacgtcaggtggcacttttcgg
ggaaatgtgcgcggaacccctatttgtttatttttctaaatacattc
aaatatgtatccgctcatgagacaataaccctgataaatgcttcaat
aatattgaaaaaggaagagtcatATGTCACTGTATCGCCGTCTAGTT
CTGCTGTCTTGTCTCTCATGGCCGCTGGCTGGCTTTTCTGCCACCGC
GCTGACCAACCTCGTCGCGGAACCATTCGCTAAACTCGAACAGGACT
TTGGCGGCTCCATCGGTGTGTACGCGATGGATACCGGCTCAGGCGCA
ACTGTAAGTTACCGCGCTGAGGAGCGCTTCCCACTGTGCAGCTCATT
CAAGGGCTTTCTTGCTGCCGCTGTGCTGGCTCGCAGCCAGCAGCAGG
CCGGCTTGCTGGACACACCCATCCGTTACGGCAAAAATGCGCTGGTT
CGGTGGTCACCCATCTCGGAAAAATATCTGACAACAGGCATGACGGT
GGCGGAGCTGTCCGCGGCCGCCGTGCAATACAGTGATAACGCCGCCG
CCAATTTGTTGCTGAAGGAGTTGGGCGGCCCGGCCGGGCTGACGGCC
TTCATGCGCTCTATCGGCGATACCACGTTCCGTCTGGACCGCTGGGA
GCTGGAGCTGAACTCCGCCATCCCAGGCGATGCGCGCGATACCTCAT
CGCCGCGCGCCGTGACGGAAAGCTTACAAAAACTGACACTGGGCTCT
GCACTGGCTGCGCCGCAGCGGCAGCAGTTTGTTGATTGGCTAAAGGG
AAACACGACCGGCAACCACCGCATCCGCGCGGCGGTGCCGGCAGACT
GGGCAGTCGGAGACAAAACCGGAACCTGCGGAGGGTATGGCACGGCA
AATGACTATGCCGTCGTCTGGCCCACTGGGCGCGCACCTATTGTGTT
GGCCGTCTACACCCGGGCGCCTAACAAGGATGACAAGCACAGCGAGG
CCGTCATCGCCGCTGCGGCTAGACTCGCGCTCGAGGGATTGGGCGTC
AACGGGCAGTAAggatccctgtcagaccaagtttactcatatatact
ttagattgatttaaaacttcatttttaatttaaaaggatctaggtga
agatcctttttgataatctcatgaccaaaatcccttaacgtgagttt
tcgttccactgagcgtcagaccccgtagaaaagatcaaaggatcttc
ttgagatcctttttttctgcgcgtaatctgctgcttgcaaacaaaaa
aaccaccgctaccagcggtggtttgtttgccggatcaagagctacca
actctttttccgaaggtaactggcttcagcagagcgcagataccaaa
tactgtccttctagtgtagccgtagttaggccaccacttcaagaact
ctgtagcaccgcctacatacctcgctctgctaatcctgttaccagtg
gctgctgccagtggcgataagtcgtgtcttaccgggttggactcaag
acgatagttaccggataaggcgcagcggtcgggctgaacggggggtt
cgtgcacacagcccagcttggagcgaacgacctacaccgaactgaga
tacctacagcgtgagctatgagaaagcgccacgcttcccgaagggag
aaaggcggacaggtatccggtaagcggcagggtcggaacaggagagc
gcacgagggagcttccagggggaaacgcctggtatctttatagtcct
gtcgggtttcgccacctctgacttgagcgtcgatttttgtgatgctc
gtcaggggggcggagcctatggaaaaacgccagcaacgcggcctttt
tacggttcctggccttttgctggccttttgctcacatgttctttcct
gcgttatcccctgattctgtggataaccgtattaccgcctttgagtg
agctgataccgctcgccgcagccgaacgaccgagcgcagcgagtcag
tgagcgaggaagcggaagagcgcctgatgcggtattttctccttacg
catctgtgcggtatttcacaccgcatttggtgcactctcagtacaat
ctgctctgatgccgcatagttaagccagtatacactccgctatcgct
acgtgactgggtcatggctgcgccccgacacccgccaacacccgctg
acgcgccctgacgggcttgtctgctcccggcatccgcttacagacaa
gctgtgaccgtctccgggagctgcatgtgtcagaggttttcaccgtc
atcaccgaaacgcgcgaggcagctgcggtaaagctcatcagcgtggt
cgtgaagcgattcacagatgtctgcctgttcatccgcgtccagctcg
ttgagtttctccagaagcgttaatgtctggcttctgataaagcgggc
catgttaagggcggttttttcctgtttggtcactgatgcctccgtgt
aagggggatttctgttcatgggggtaatgataccgatgaaacgagag
aggatgctcacgatacgggttactgatgatgaacatgcccggttact
ggaacgttgtgagggtaaacaactggcggtatggatgcggcgggacc
agagaaaaatcactcagggtcaatgccagcgcttcgttaatacagat
gtaggtgttccacagggtagccagcagcatcctgcgatgcagatccg
gaacataatggtgcagggcgctgacttccgcgtttccagactttacg
aaacacggaaaccgaagaccattcatgttgttgctcaggtcgcagac
gttttgcagcagcagtcgcttcacgttcgctcgcgtatcggtgattc
attctgctaaccagtaaggcaaccccgccagcctagccgggtcctca
acgacaggagcacgatcatgcgcacccgtggccaggacccaacgctg
cccgagatgcgccgcgtgcggctgctggagatggcggacgcgatgga
tatgttctgccaagggttggtttgcgcattcacagttctccgcaaga
attgattggctccaattcttggagtggtgaatccgttagcgaggtgc
cgccggcttccattcaggtcgaggtggcccggctccatgcaccgcga
cgcaacgcggggaggcagacaaggtatagggcggcgcctacaatcca
tgccaacccgttccatgtgctcgccgaggcggcataaatcgccgtga
cgatcagcggtccagtgatcgaagttaggctggtaagagccgcgagc
gatccttgaagctgtccctgatggtcgtcatctacctgcctgcctgg
acagcatggcctgcaacgcgggcatcccgatgccgccggaagcgaga
agaatcataatggggaaggccatccagcctcgcgtcgcgaacgccag
caagacgtagcccagcgcgtcggccgccatgccggcgataatggcct
gcttctcgccgaaacgtttggtggcgggaccagtgacgaaggcttga
gcgagggcgtgcaagattccgaataccgcaagcgacaggccgatcat
cgtcgcgctccagcgaaagcggtcctcgccgaaaatgacccagagcg
ctgccggcacctgtcctacgagttgcatgataaagaagacagtcata
agtgcggcgacgatagtcatgccccgcgcccaccggaaggagctgac
tgggttgaaggctctcaagggcatcggtcgacgctctcccttatgcg
actcctgcattaggaagcagcccagtagtaggttgaggccgttgagc
accgccgccgcaaggaatggtgcatgcaaggagatggcgcccaacag
tcccccggccacggggcctgccaccatacccacgccgaaacaagcgc
tcatgagcccgaagtggcgagcccgatcttccccatcggtgatgtcg
gcgatataggcgccagcaaccgcacctgtggcgccggtgatgccggc
cacgatgcgtccggcgtagaggattcacaggacgggtgtggtcgcca
tgatcgcgtagtcgatagtggctccaagtagcgaagcgagcaggact
gggcggcggccaaagcggtcggacagtgctccgagaacgggtgcgca
tagaaattgcatcaacgcatatagcgctagcagcacgccatagtgac
tggcgatgctgtcggaatggacgatatcccgcaagaggcccggcagt
accggcataaccaagcctatgcctacagcatccagggtgacggtgcc
gaggatgacgatgagcgcattgttagatttcatacacggtgcctgac
tgcgttagcaatttaactgtgataaactaccgcattaaagcttatcg
atgataagctgtcaaacatgagaa
pBR-CBST-DHA-1
SEQ ID 12
ttcttgaagacgaaagggcctcgtgatacgcctatttttataggtta
atgtcatgataataatggtttcttagacgtcaggtggcacttttcgg
ggaaatgtgcgcggaacccctatttgtttatttttctaaatacattc
aaatatgtatccgctcatgagacaataaccctgataaatgcttcaat
aatattgaaaaaggaagagtcatATGAAAAAATCGTTATCTGCAACA
CTGATTTCCGCTCTGCTGGCGTTTTCCGCCCCGGGGTTTTCTGCCGC
TGATAATGTCGCGGCGGTGGTGGACAGCACCATTAAACCGCTGATGG
CACAGCAGGATATTCCCGGGATGGCGGTTGCCGTCTCCGTAAAGGGT
AAGCCCTATTATTTCAATTATGGTTTTGCCGATATTCAGGCAAAACA
GCCGGTCACTGAAAATACACTATTTGAGCTCGGATCTGTAAGTAAAA
CTTTCACAGGTGTGCTGGGTGCGGTTTCTGTGGCGAAAAAAGAGATG
GCGCTGAATGATCCGGCGGCAAAATACCAGCCGGAGCTGGCTCTGCC
GCAGTGGAAGGGGATCACATTGCTGGATCTGGCTACCTATACCGCAG
GCGGACTGCCGTTACAGGTGCCGGATGCGGTAAAAAGCCGTGCGGAT
CTGCTGAATTTCTATCAGCAGTGGCAGCCGTCCCGGAAACCGGGCGA
TATGCGTCTGTATGCAAACAGCAGTATCGGCCTGTTTGGTGCTCTGA
CCGCAAACGCGGCGGGGATGCCGTATGAGCAGTTGCTGACTGCACGC
ATCCTGGCACCGCTGGGGTTATCTCACACCTTTATTACTGTGCCGGA
AAGTGCGCAAAGCCAGTATGCGTACGGTTATAAAAACAAAAAACCGG
TCCGCGTGTCGCCGGGACAGCTTGATGCGGAATCTTACGGCGTGAAA
TCCGCCTCAAAAGATATGCTGCGCTGGGCGGAAATGAATATGGAGCC
GTCACGGGCCGGTAATGCGGATCTGGAAATGGCAATGTATCTCGCCC
AGACCCGCTACTATAAAACCGCCGCGATTAACCAGGGGCTGGGCTGG
GAAATGTATGACTGGCCGCAGCAGAAAGATATGATCATTAACGGTGT
GACCAACGAGGTCGCATTGCAGCCGCATCCGGTAACAGACAACCAGG
TTCAGCCGTATAACCGTGCTTCCTGGGTGCATAAAACGGGCGCAACA
ACTGGTTTCGGCGCCTATGTCGCCTTTATTCCGGAAAAACAGGTGGC
GATTGTGATTCTGGCGAATAAAAACTACCCGAATACCGAAAGAGTCA
AAGCTGCACAGGCTATTTTGAGTGCACTGGAATAAggatccctgtca
gaccaagtttactcatatatactttagattgatttaaaacttcattt
ttaatttaaaaggatctaggtgaagatcctttttgataatctcatga
ccaaaatcccttaacgtgagttttcgttccactgagcgtcagacccc
gtagaaaagatcaaaggatcttcttgagatcctttttttctgcgcgt
aatctgctgcttgcaaacaaaaaaaccaccgctaccagcggtggttt
gtttgccggatcaagagctaccaactctttttccgaaggtaactggc
ttcagcagagcgcagataccaaatactgtccttctagtgtagccgta
gttaggccaccacttcaagaactctgtagcaccgcctacatacctcg
ctctgctaatcctgttaccagtggctgctgccagtggcgataagtcg
tgtcttaccgggttggactcaagacgatagttaccggataaggcgca
gcggtcgggctgaacggggggttcgtgcacacagcccagcttggagc
gaacgacctacaccgaactgagatacctacagcgtgagctatgagaa
agcgccacgcttcccgaagggagaaaggcggacaggtatccggtaag
cggcagggtcggaacaggagagcgcacgagggagcttccagggggaa
acgcctggtatctttatagtcctgtcgggtttcgccacctctgactt
gagcgtcgatttttgtgatgctcgtcaggggggcggagcctatggaa
aaacgccagcaacgcggcctttttacggttcctggccttttgctggc
cttttgctcacatgttctttcctgcgttatcccctgattctgtggat
aaccgtattaccgcctttgagtgagctgataccgctcgccgcagccg
aacgaccgagcgcagcgagtcagtgagcgaggaagcggaagagcgcc
tgatgcggtattttctccttacgcatctgtgcggtatttcacaccgc
atttggtgcactctcagtacaatctgctctgatgccgcatagttaag
ccagtatacactccgctatcgctacgtgactgggtcatggctgcgcc
ccgacacccgccaacacccgctgacgcgccctgacgggcttgtctgc
tcccggcatccgcttacagacaagctgtgaccgtctccgggagctgc
atgtgtcagaggttttcaccgtcatcaccgaaacgcgcgaggcagct
gcggtaaagctcatcagcgtggtcgtgaagcgattcacagatgtctg
cctgttcatccgcgtccagctcgttgagtttctccagaagcgttaat
gtctggcttctgataaagcgggccatgttaagggcggttttttcctg
tttggtcactgatgcctccgtgtaagggggatttctgttcatggggg
taatgataccgatgaaacgagagaggatgctcacgatacgggttact
gatgatgaacatgcccggttactggaacgttgtgagggtaaacaact
ggcggtatggatgcggcgggaccagagaaaaatcactcagggtcaat
gccagcgcttcgttaatacagatgtaggtgttccacagggtagccag
cagcatcctgcgatgcagatccggaacataatggtgcagggcgctga
cttccgcgtttccagactttacgaaacacggaaaccgaagaccattc
atgttgttgctcaggtcgcagacgttttgcagcagcagtcgcttcac
gttcgctcgcgtatcggtgattcattctgctaaccagtaaggcaacc
ccgccagcctagccgggtcctcaacgacaggagcacgatcatgcgca
cccgtggccaggacccaacgctgcccgagatgcgccgcgtgcggctg
ctggagatggcggacgcgatggatatgttctgccaagggttggtttg
cgcattcacagttctccgcaagaattgattggctccaattcttggag
tggtgaatccgttagcgaggtgccgccggcttccattcaggtcgagg
tggcccggctccatgcaccgcgacgcaacgcggggaggcagacaagg
tatagggcggcgcctacaatccatgccaacccgttccatgtgctcgc
cgaggcggcataaatcgccgtgacgatcagcggtccagtgatcgaag
ttaggatggtaagagccgcgagcgatccttgaagctgtccctgatgg
tcgtcatctacctgcctggacagcatggcctgcaacgcgggcatccc
gatgccgccggaagcgagaagaatcataatggggaaggccatccagc
ctcgcgtcgcgaacgccagcaagacgtagcccagcgcgtcggccgcc
atgccggcgataatggcctgcttctcgccgaaacgtttggtggcggg
accagtgacgaaggcttgagcgagggcgtgcaagattccgaataccg
caagcgacaggccgatcatcgtcgcgctccagcgaaagcggtcctcg
ccgaaaatgacccagagcgctgccggcacctgtcctacgagttgcat
gataaagaagacagtcataagtgcggcgacgatagtcatgccccgcg
cccaccggaaggagctgactgggttgaaggctctcaagggcatcggt
cgacgctctcccttatgcgactcctgcattaggaagcagcccagtag
taggttgaggccgttgagcaccgccgccgcaaggaatggtgcatgca
aggagatggcgcccaacagtcccccggccacggggcctgccaccata
cccacgccgaaacaagcgctcatgagcccgaagtggcgagcccgatc
ttccccatcggtgatgtcggcgatataggcgccagcaaccgcacctg
tggcgccggtgatgccggccacgatgcgtccggcgtagaggattcac
aggacgggtgtggtcgccatgatcgcgtagtcgatagtggctccaag
tagcgaagcgagcaggactgggcggcggccaaagcggtcggacagtg
ctccgagaacgggtgcgcatagaaattgcatcaacgcatatagcgct
agcagcacgccatagtgactggcgatgctgtcggaatggacgatatc
ccgcaagaggcccggcagtaccggcataaccaagcctatgcctacag
catccagggtgacggtgccgaggatgacgatgagcgcattgttagat
ttcatacacggtgcctgactgcgttagcaatttaactgtgataaact
accgcattaaagcttatcgatgataagctgtcaaacatgagaa
pBR-CBST-ADC-33
SEQ ID 13
ttcttgaagacgaaagggcctcgtgatacgcctatttttataggtta
atgtcatgataataatggtttcttagacgtcaggtggcacttttcgg
ggaaatgtgcgcggaacccctatttgtttatttttctaaatacattc
aaatatgtatccgctcatgagacaataaccctgataaatgcttcaat
aatattgaaaaaggaagagtcatATGCGATTTAAAAAAATTTCTTGT
CTACTTTTATCCCCGCTTTTTATTTTTAGTACCTCAATTTATGCGGG
CAATACACCAAAAGACCAAGAAATTAAAAAACTGGTAGATCAAAACT
TTAAACCGTTATTAGAAAAATATGATGTGCCAGGTATGGCTGTGGGT
GTTATTCAAAATAATAAAAAGTATGAAATGTATTATGGTCTTCAATC
TGTTCAAGATAAAAAAGCCGTAAATAGCAGTACCATTTTTGAGCTAG
GTTCTGTCAGTAAATTATTTACCGCGACAGCAGGTGGATATGCAAAA
AATAAAGGAAAAATCTCTTTTGACGATACGCCTGGTAAATATTGGAA
AGAACTAAAAAACACACCGATTGACCAAGTTAACTTACTTCAACTCG
CGACGTATACAAGTGGTAACCTTGCCTTGCAGTTTCCAGATGAAGTA
AAAACAGACCAACAAGTTTTAACTTTTTTCAAAGACTGGAAACCTAA
AAACTCAATCGGTGAATACAGACAATATTCAAATCCAAGTATTGGCC
TATTTGGAAAGGTTGTGGCTTTGTCTATGAATAAACCTTTCGACCAA
GTCTTAGAAAAAACAATTTTTCCGGCCCTTGGCTTAAAACATAGCTA
TGTAAATGTACCTAAGACCCAGATGCAAAACTATGCATTTGGTTATA
ACCAAGAAAATCAGCCGATTCGAGTTAACCGCGGCCCACTCGATGCC
GCCCCTGCGTATGGCGTCAAATCGACACTACCCGACATGTTGAGTTT
TATTCATGCCAACCTTAACCCACAGAAATATCCGGCTGATATTCAAC
GGGCAATTAATGAAACACATCAAGGGCGCTATCAAGTAAATACCATG
TATCAGGCACTCGGTTGGGAAGAGTTTTCTTATCCGGCAACGTTACA
AACTTTATTAGACAGTAATTCAGAACAGATTGTGATGAAACCTAATA
AAGTGACTGCTATTTCAAAGGAACCTTCAGTTAAGATGTACCATAAA
ACTGGCTCAACCAACGGTTTCGGAACGTATGTAGTGTTTATTCCTAA
AGAAAATATTGGCTTAGTCATGTTAACCAATAAACGTATTCCAAATG
AAGAGCGCATTAAGGCAGCTTATGCTGTGCTGAATGCAATAAAGAAA
TAAggatccctgtcagaccaagtttactcatatatactttagattga
tttaaaacttcatttttaatttaaaaggatctaggtgaagatccttt
ttgataatctcatgaccaaaatcccttaacgtgagttttcgttccac
tgagcgtcagaccccgtagaaaagatcaaaggatcttcttgagatcc
tttttttctgcgcgtaatctgctgcttgcaaacaaaaaaaccaccgc
taccagcggtggtttgtttgccggatcaagagctaccaactcttttt
ccgaaggtaactggcttcagcagagcgcagataccaaatactgtcct
tctagtgtagccgtagttaggccaccacttcaagaactctgtagcac
cgcctacatacctcgctctgctaatcctgttaccagtggctgctgcc
agtggcgataagtcgtgtcttaccgggttggactcaagacgatagtt
accggataaggcgcagcggtcgggctgaacggggggttcgtgcacac
agcccagcttggagcgaacgacctacaccgaactgagatacctacag
cgtgagctatgagaaagcgccacgcttcccgaagggagaaaggcgga
caggtatccggtaagcggcagggtcggaacaggagagcgcacgaggg
agcttccagggggaaacgcctggtatctttatagtcctgtcgggttt
cgccacctctgacttgagcgtcgatttttgtgatgctcgtcaggggg
gcggagcctatggaaaaacgccagcaacgcggcctttttacggttcc
tggccttttgctggccttttgctcacatgttctttcctgcgttatcc
cctgattctgtggataaccgtattaccgcctttgagtgagctgatac
cgctcgccgcagccgaacgaccgagcgcagcgagtcagtgagcgagg
aagcggaagagcgcctgatgcggtattttctccttacgcatctgtgc
ggtatttcacaccgcatttggtgcactctcagtacaatctgctctga
tgccgcatagttaagccagtatacactccgctatcgctacgtgactg
ggtcatggctgcgccccgacacccgccaacacccgctgacgcgccct
gacgggcttgtctgctcccggcatccgcttacagacaagctgtgacc
gtctccgggagctgcatgtgtcagaggttttcaccgtcatcaccgaa
acgcgcgaggcagctgcggtaaagctcatcagcgtggtcgtgaagcg
attcacagatgtctgcctgttcatccgcgtccagctcgttgagtttc
tccagaagcgttaatgtctggcttctgataaagcgggccatgttaag
ggcggttttttcctgtttggtcactgatgcctccgtgtaagggggat
ttctgttcatgggggtaatgataccgatgaaacgagagaggatgctc
acgatacgggttactgatgatgaacatgcccggttactggaacgttg
tgagggtaaacaactggcggtatggatgcggcgggaccagagaaaaa
tcactcagggtcaatgccagcgcttcgttaatacagatgtaggtgtt
ccacagggtagccagcagcatcctgcgatgcagatccggaacataat
ggtgcagggcgctgacttccgcgtttccagactttacgaaacacgga
aaccgaagaccattcatgttgttgctcaggtcgcagacgttttgcag
cagcagtcgcttcacgttcgctcgcgtatcggtgattcattctgcta
accagtaaggcaaccccgccagcctagccgggtcctcaacgacagga
gcacgatcatgcgcacccgtggccaggacccaacgctgcccgagatg
cgccgcgtgcggctgctggagatggcggacgcgatggatatgttctg
ccaagggttggtttgcgcattcacagttctccgcaagaattgattgg
ctccaattcttggagtggtgaatccgttagcgaggtgccgccggctt
ccattcaggtcgaggtggcccggctccatgcaccgcgacgcaacgcg
gggaggcagacaaggtatagggcggcgcctacaatccatgccaaccc
gttccatgtgctcgccgaggcggcataaatcgccgtgacgatcagcg
gtccagtgatcgaagttaggctggtaagagccgcgagcgatccttga
agctgtccctgatggtcgtcatctacctgcctggacagcatggcctg
caacgcgggcatcccgatgccgccggaagcgagaagaatcataatgg
ggaaggccatccagcctcgcgtcgcgaacgccagcaagacgtagccc
agcgcgtcggccgccatgccggcgataatggcctgcuctcgccgaaa
cgtttggtggcgggaccagtgacgaaggcttgagcgagggcgtgcaa
gattccgaataccgcaagcgacaggccgatcatcgtcgcgctccagc
gaaagcggtcctcgccgaaaatgacccagagcgctgccggcacctgt
cctacgagttgcatgataaagaagacagtcataagtgcggcgacgat
agtcatgccccgcgcccaccggaaggagctgactgggugaaggctct
caagggcatcggtcgacgctctcccttatgcgactcctgcattagga
agcagcccagtagtaggttgaggccgttgagcaccgccgccgcaagg
aatggtgcatgcaaggagatggcgcccaacagtcccccggccacggg
gcctgccaccatacccacgccgaaacaagcgctcatgagcccgaagt
ggcgagcccgatcttccccatcggtgatgtcggcgatataggcgcca
gcaaccgcacctgtggcgccggtgatgccggccacgatgcgtccggc
gtagaggattcacaggacgggtgtggtcgccatgatcgcgtagtcga
tagtggctccaagtagcgaagcgagcaggactgggcggcggccaaag
cggtcggacagtgctccgagaacgggtgcgcatagaaattgcatcaa
cgcatatagcgctagcagcacgccatagtgactggcgatgctgtcgg
aatggacgatatcccgcaagaggcccggcagtaccggcataaccaag
cctatgcctacagcatccagggtgacggtgccgaggatgacgatgag
cgcattgttagatttcatacacggtgcctgactgcgttagcaattta
actgtgataaactaccgcattaaagcttatcgatgataagctgtcaa
acatgagaa

### Example 35

### Standard BLI Potentiation MIC Assay

The ability of compounds to potentiate the activity of β-lactams was demonstrated by determining the minimum inhibitory concentrations (MIC) of β-lactam and BLI compound combinations against various β-lactamase producing bacterial strains using the broth microdilution method. The experimental protocol was performed according to Clinical and Laboratory Standards Institute (CLSI) guidelines with modifications as described below (CLSI guidelines can be derived from the CLSI document M07-A9 published in January 2012: “Methods for Dilution Antimicrobial Susceptibility Tests for Bacteria That Grow Aerobically; Approved Standard-Ninth Edition”).

To prepare for MIC testing, frozen glycerol stocks of clinical isolates (Klebsiella pneumoniae, Eschericia coli, Enterobacter spp, Citrobacter spp, or Pseudomonas aeruginosa) were used to streak for isolated colonies on rich, non-selective, tryptic soy agar containing 5% sheep's blood (TSAB). Frozen glycerol stocks of laboratory engineered, isogenic E. coli strains, which contain cloned β-lactamase expressing plasmids were used to streak for isolated colonies on rich, selective LB agar supplemented with 25 μg/mL tetracycline to maintain the plasmid. All strains were incubated at 37° C. for 18-24 hrs.

On the day of testing, primary cultures were started by scraping off 5-10 colonies from the TSAB plates containing clinical strains or the tetracycline supplemented LB plates containing engineered strains. The clinical strain material was suspended in ˜5 mL of cation adjusted Mueller Hinton Broth (CAMHB) in 14 mL culture tubes. The engineered strain material was suspended in CAMHB (supplemented with 25 μg/mL tetracycline) in 14 mL culture tubes. All strains were incubated at 37° C. with aeration (200 rpm) for ˜2 hrs until the optical density at 600 nm (OD600) was ≧0.1.

The two compound components of the assay were each diluted in CAMHB and added to the 96-well broth microdilution assay plates. 50 μL of the β-lactam was added to each well of the assay plate in 2-fold dilutions with final concentrations ranging from 128 to 0.13 μg/mL. 25 μL of the BLI compound was added to all wells in the broth microdilution plates at a final concentration of 4 μg/mL. Inoculum cultures were prepared by standardizing the primary cultures to OD600=0.1 and then adding 20 μL of the adjusted primary culture per 1 mL CAMHB for clinical strains or CAMHB (supplemented with tetracycline at 100 μg/mL) for engineered strains, so that the final inoculum density was ˜10⁵ colony forming units per milliliter. Diluted inoculum cultures were used to inoculate 25 μL per well in 96-well broth microdilution assay plates. The final volume of each well was 100 μL and contained a β-lactam at different concentrations, a BLIcompound at 4 μg/mL concentration, the bacterial culture at an OD600 of approximately 0.001 and when necessary tetracycline at 25 μg/mL.

Plates were incubated for 18-20 hours at 37° C. with aeration (200 rpm). Following incubation, growth was confirmed visually placing plates over a viewing apparatus (stand with a mirror underneath) and then OD600 was measured using a SpectraMax 340PC384 plate reader (Molecular Devices, Sunnyvale, Calif.). Growth was defined as turbidity that could be detected with the naked eye or achieving minimum OD600 of 0.1. MIC values were defined as the lowest concentration producing no visible turbidity.

MIC values of representative compounds are shown in Table II.

### Example 36

### Synergy MIC (sMIC) Assay

The synergy MIC (sMIC) assay determines the concentration of the BLI required to potentiate the activity of a fixed concentration of a β-lactam antibiotic against β-lactamase producing bacterial strains. The experimental protocol was performed according to Clinical and Laboratory Standards Institute (CLSI) guidelines with modifications as described below (CLSI guidelines can be derived from the CLSI document M07-A9 published in January 2012: “Methods for Dilution Antimicrobial Susceptibility Tests for Bacteria That Grow Aerobically; Approved Standard-Ninth Edition”). The assay is set-up by serially diluting the BLI across 11 of the 12 wells in each row of a 96-well broth microdilution assay plate, adding the β-lactam at a fixed concentration to all wells in the assay plate, inoculating the assay plate with bacterial strains, and determining the lowest concentration of BLI required to inhibit overnight bacterial growth. Bacterial growth in the 12^{th} well of the assay plate, which contains the β-lactam at a fixed concentration but does not contain any BLI, demonstrates that the bacterial strains are resistant to the β-lactam antibiotic (e.g. ceftolozane) at the fixed concentration of 4 μg/mL.

To prepare for MIC testing, frozen glycerol stocks of clinical isolates (Klebsiella pneumoniae, Eschericia coli, Enterobacter spp, Citrobacter spp, or Pseudomonas aeruginosa) were used to streak for isolated colonies on rich, non-selective, tryptic soy agar containing 5% sheep's blood (TSAB). Frozen glycerol stocks of laboratory engineered, isogenic E. coli strains, which contain cloned β-lactamase expressing plasmids were used to streak for isolated colonies on rich, selective LB agar supplemented with 25 μg/mL tetracycline to maintain the plasmid. All strains were incubated at 37° C. for 18-24 hrs.

On the day of testing, primary cultures were started by scraping off 5-10 colonies from the TSAB plates containing clinical strains or the tetracycline supplemented LB plates containing engineered strains. The clinical strain material was suspended in ˜5 mL of cation adjusted Mueller Hinton Broth (CAMHB) in 14 mL culture tubes. The engineered strain material was suspended in CAMHB (supplemented with tetracycline at 25 μg/mL) in 14 mL culture tubes. All strains were incubated at 37° C. with aeration (200 rpm) for ˜2 hrs until the OD600 was ≧0.1.

The two compound components of the assay were each prepared in CAMHB and added to the 96-well broth microdilution assay plates. 50 μL of the BLI was added to each well of the assay plate in 2-fold dilutions with final concentrations ranging from 128 to 0.13 μg/mL. 25 μL of the β-lactam was added to all wells in the broth microdilution plates at a final concentration of 4 μg/mL. Inoculum cultures were prepared by standardizing the primary cultures to OD600=0.1 and then adding 20 μL of the adjusted primary culture per 1 mL CAMHB for clinical strains or CAMHB (supplemented with tetracycline at 100 μg/mL) for isogenic strains, so that the final inoculum density was ˜10⁵ colony forming units per milliliter. Diluted inoculum cultures were used to inoculate 25 μL per well in 96-well broth microdilution assay plates. The final volume of each well was 100 μL and contained a BLI at different concentrations, a β-lactam at 4 μg/mL concentration, the bacterial culture at an OD600 of approximately 0.001 and when necessary tetracycline at 25 ug/mL.

Interpreting the sMIC Data:

Plates were incubated for 18-20 hours at 37° C. with aeration (200 rpm). Following incubation, growth was confirmed visually placing plates over a viewing apparatus (stand with a mirror underneath) and then OD600 was measured using a SpectraMax 340PC384 plate reader (Molecular Devices, Sunnyvale, Calif.). Growth was defined as turbidity that could be detected with the naked eye or achieving minimum OD600 of 0.1. sMIC values were defined as the lowest concentration producing no visible turbidity.

The sMIC values represent the amount of BLI required to potentiate the activity of 4 μg/ml of CXA-101 (Ceftolozane) or ceftazidime to inhibit the growth of the β-lactamase producing bacteria.

sMIC values of representative compounds are shown in Table III.

### Example 37

### Inhibition Kinetics

Inhibition or inactivation of KPC-2 by test inhibitors was assessed using 100 μM nitrocefin (NCF) as a reporter substrate. Assays were performed in 1× PBS pH 7.4, 0.1 mg/ml BSA, in 96-well half area plates, 50 μl reaction volume. NCF was dissolved in DMSO and diluted in assay buffer. Test inhibitors were dissolved in water or DMSO and serially diluted in the assay with final concentrations between 2000-0.195 μM. The enzyme activity in the presence of varying concentrations of test inhibitor was determined by monitoring the hydrolysis of NCF spectrophotometrically at 486 nm, for 5 minutes, 25° C., using a SpectraMax Plus384 microplate reader with SoftMax Pro software (Molecular Devices). Data analysis was performed using GraphPad Prism (GraphPad Software, Inc.).

Progress curves were fit to a first-order rate decay equation (Eq. 1) to determine k_{observed} (k_{obs}).

k_{obs} vs. inhibitor concentration [I] curves were then fit to Eq.2 to determine the inhibitor dissociation constant (K) and the first order rate constant of enzyme inactivation at infinite inhibitor concentration (k_{inact}). Table IV shows kinetics results from representative test compounds. A larger k_{inact}/K ratio indicates a more effective enzyme inactivator.

Yₜ=V₀*(1−e^{(-k}^{_{obs}}*^{t)})/k_{obs} Eq. 1

Where Y is the absorbance at time t, V₀ is the uninhibited enzyme velocity, k_{obs} is the observed rate constant of the enzyme inactivation.

k_{obs}=k_{inact}*[I]/([I]+K(1+S/Kₘ)) Eq. 2

Where S is the NCF concentration, Kₘ is the KPC-2 Kₘ for NCF

1.-50. (canceled)

51. A pharmaceutical composition comprising aztreonam or a pharmaceutically acceptable salt thereof and a compound of Formula (A-I) or a pharmaceutically acceptable salt thereof: 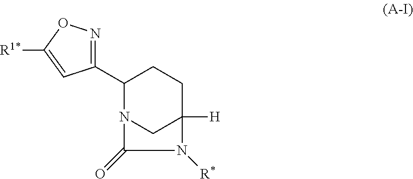
wherein
R* is selected from 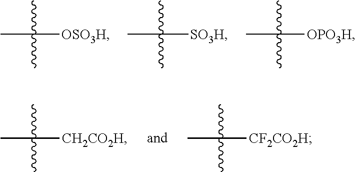
and
R¹* is selected from:
a. 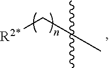
wherein R²* is selected from 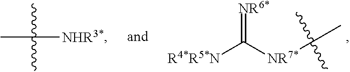
R³* is selected from hydrogen, (C₁-C₃)-alkyl, aminoalkyl, aminocycloalkyl, hydroxyalkyl, 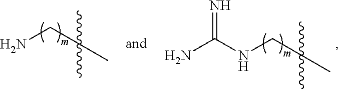
each of R⁴*, R⁵*, R⁶* and R⁷* is independently selected from hydrogen, (C₁-C₆)-alkyl, aminoalkyl, aminocycloalkyl, and hydroxyalkyl, provided that at least one of R⁴*, R⁵*, R⁶* and R⁷* is hydrogen,
n is selected from 1, 2, 3 and 4, and
m is selected from 1, 2 and 3;
b. 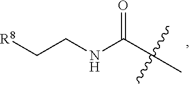
wherein R⁸ is selected from —NH(C₁-C₃)-alkyl and 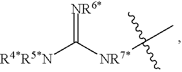
wherein each of R⁴*, R⁵*, R⁶* and R⁷* is as described previously;
c. 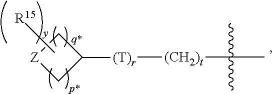
wherein Z is selected from CR⁹R¹⁰ and NR¹¹,
each of R⁹ and R¹⁰ is independently selected from H, NH₂, —NH(C₁-C₃)-alkyl and 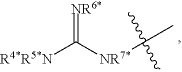
wherein each of R⁴*, R⁵*, R⁶* and R⁷* is as described previously,
alternatively, R⁹ and R¹⁰ together with the carbon to which they are attached, form a cycloalkyl or heterocyclyl ring containing 4-6 ring members,
R¹¹ is selected from H and 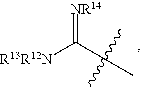
each of R¹², R¹³ and R¹⁴ is independently selected from hydrogen, (C₁-C₆)-alkyl, amino alkyl, aminocycloalkyl, and hydroxyalkyl, provided that at least one of R¹², R¹³ and R¹⁴ is hydrogen,
R¹⁵ is selected from NH₂ and 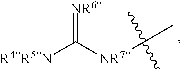
wherein each of R⁴*, R⁵*, R⁶* and R⁷* is as described previously,
each of p* and q* is independently selected from 0, 1, 2 and 3,
T is selected from NH and O,
t is selected from 0, 1, 2, 3, and 4, and
each of r and y is independently selected from 0 and 1;
d. 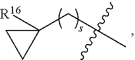
wherein R¹⁶ is selected from NH₂, —NH(C₁-C₃)-alkyl and 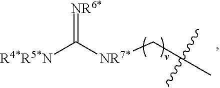
wherein each of R⁴*, R⁵*, R⁶* and R⁷* is as described previously,
s is selected from 0 and 1, and,
v is selected from 0, 1, 2, and 3;
e. 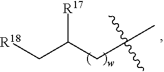
wherein R¹⁸ is selected from NH₂ and 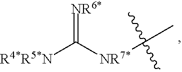
wherein each of R⁴*, R⁵*, R⁶* and R⁷* is as described previously,
R¹⁷ is selected from amino and hydroxyl, and
w is selected from 0 and 1;
f. 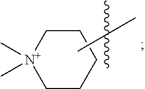 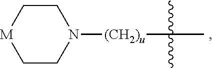
wherein M is selected from NR¹⁹, CR²⁰R²¹ and O,
wherein R¹⁹ is selected from H and 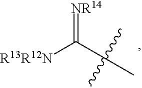
where each of R¹², R¹³ and R¹⁴ is as described previously,
each of R²⁰ and R²¹ is independently selected from H, NH₂ and 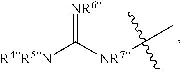
wherein each of R⁴*, R⁵*, R⁶* and R⁷* is as described previously, and
u is selected from 0, 1 and 2; and
g.

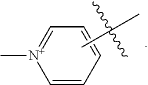

52. The pharmaceutical composition according to claim 51, wherein the compound of Formula A-I has the stereochemistry specified in Formula A-II 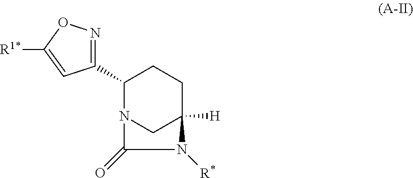
or a pharmaceutically acceptable salt thereof.

53. The pharmaceutical composition according to claim 52, wherein the compound, or a pharmaceutically acceptable salt thereof is selected from

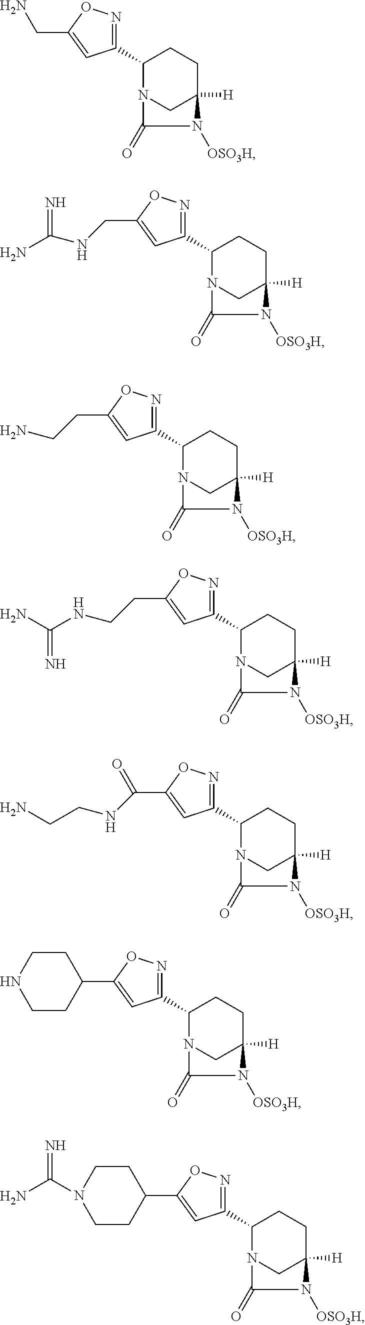

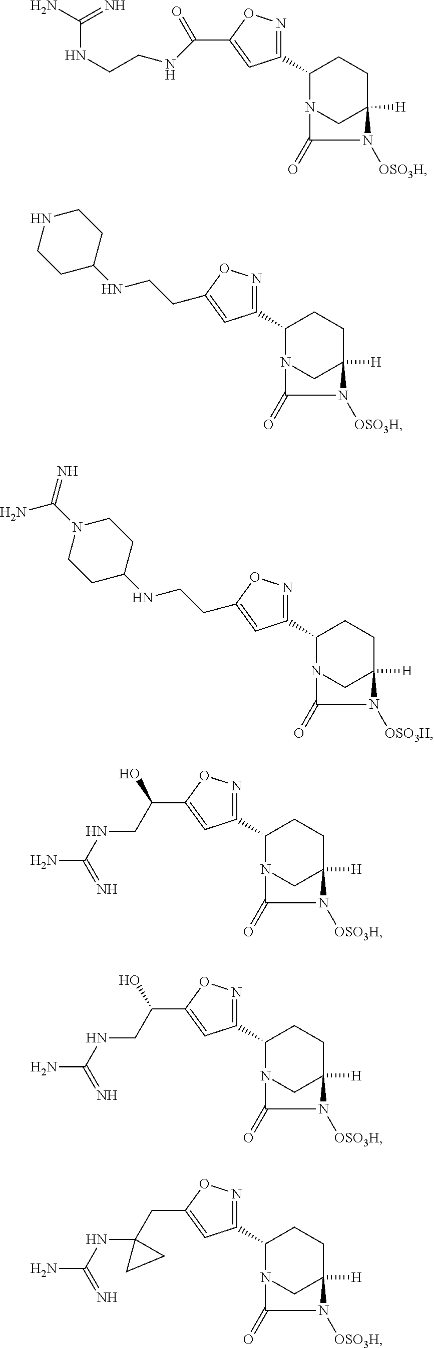

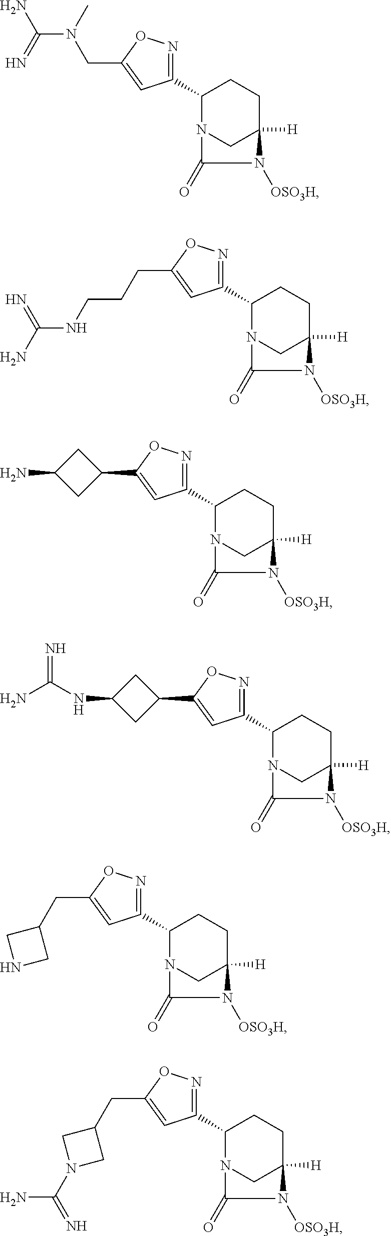

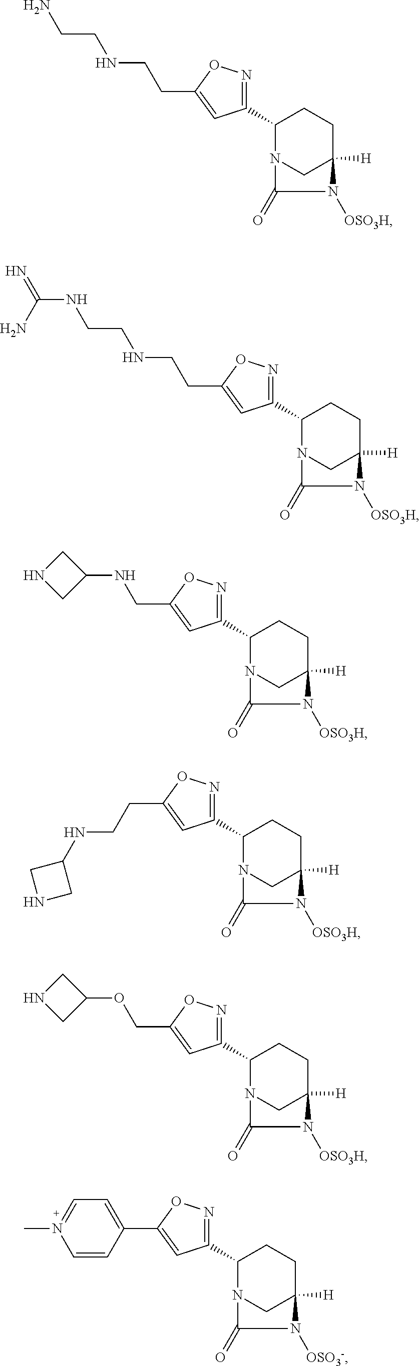

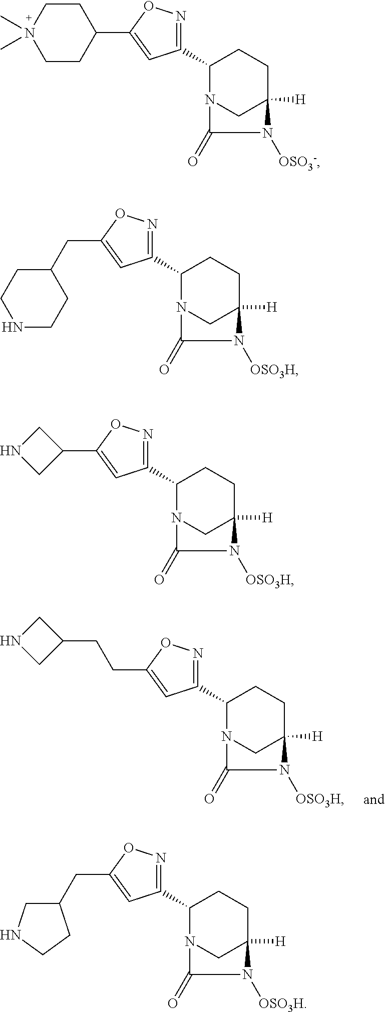

54. The pharmaceutical composition according to claim 52, wherein R¹* is selected from

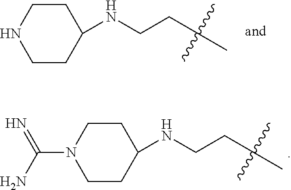 [SMILES: *CCNC1CCNCC1 ; *CCNC1CCN(C(=N)N)CC1]

55. The pharmaceutical composition according to claim 54, wherein the compound is selected from

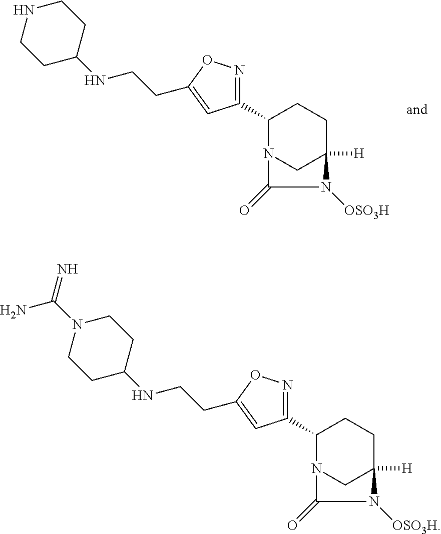

56. The pharmaceutical composition according to claim 55, wherein the compound or a pharmaceutically acceptable salt thereof has the Formula

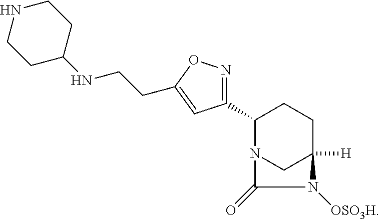

57. The pharmaceutical composition compound according to claim 55, wherein the compound or a pharmaceutically acceptable salt thereof has the Formula

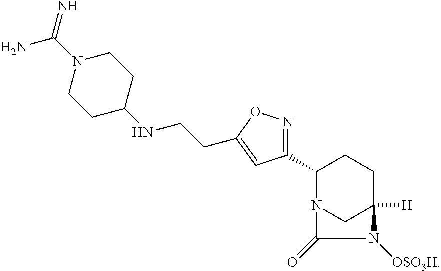

58. A pharmaceutical composition comprising meropenem or a pharmaceutically acceptable salt thereof and a compound of Formula (A-I) or a pharmaceutically acceptable salt thereof: 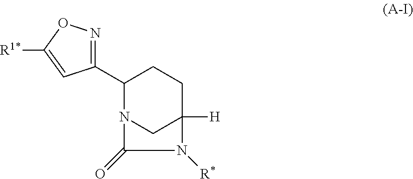
wherein
R* is selected from 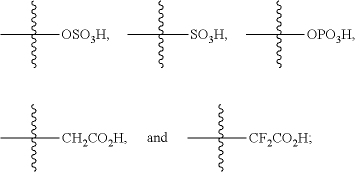
and
R¹* is selected from:
a. 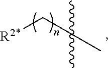
wherein R²* is selected from 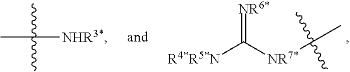
R³* is selected from hydrogen, (C₁-C₃)-alkyl, aminoalkyl, aminocycloalkyl, hydroxyalkyl, 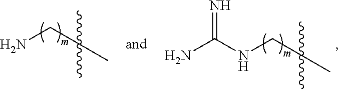
each of R⁴*, R⁵*, R⁶* and R⁷* is independently selected from hydrogen, (C₁-C₆)-alkyl, aminoalkyl, aminocycloalkyl, and hydroxyalkyl, provided that at least one of R⁴*, R⁵*, R⁶* and R⁷* is hydrogen,
n is selected from 1, 2, 3 and 4, and
m is selected from 1, 2 and 3;
b. 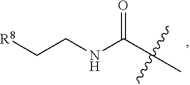
wherein R⁸ is selected from —NH(C₁-C₃)-alkyl and 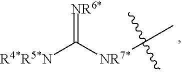
wherein each of R⁴*, R⁵*, R⁶* and R⁷* is as described previously;
c. 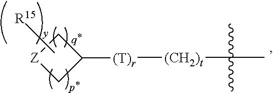
wherein Z is selected from CR⁹R¹⁰ and NR¹¹,
each of R⁹ and R¹⁰ is independently selected from H, NH₂, —NH(C₁-C₃)-alkyl and 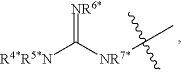
wherein each of R⁴*, R⁵*, R⁶* and R⁷* is as described previously,
alternatively, R⁹ and R¹⁰ together with the carbon to which they are attached, form a cycloalkyl or heterocyclyl ring containing 4-6 ring members,
R¹¹ is selected from H and 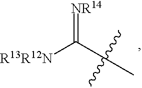
each of R¹², R¹³ and R¹⁴ is independently selected from hydrogen, (C₁-C₆)-alkyl, amino alkyl, aminocycloalkyl, and hydroxyalkyl, provided that at least one of R¹², R¹³ and R¹⁴ is hydrogen,
R¹⁵ is selected from NH₂ and 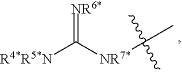
wherein each of R⁴*, R⁵*, R⁶* and R⁷* is as described previously,
each of p* and q* is independently selected from 0, 1, 2 and 3,
T is selected from NH and O,
t is selected from 0, 1, 2, 3, and 4, and
each of r and y is independently selected from 0 and 1;
d. 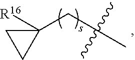
wherein R¹⁶ is selected from NH₂, —NH(C₁-C₃)-alkyl and 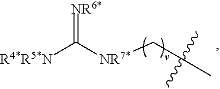
wherein each of R⁴*, R⁵*, R⁶* and R⁷* is as described previously,
s is selected from 0 and 1, and,
v is selected from 0, 1, 2, and 3;
e. 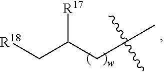
wherein R¹⁸ is selected from NH₂ and 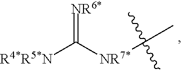
wherein each of R⁴*, R⁵*, R⁶* and R⁷* is as described previously,
R¹⁷ is selected from amino and hydroxyl, and
w is selected from 0 and 1;
f. 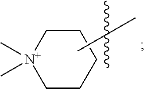 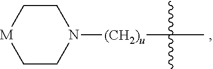
wherein M is selected from NR¹⁹, CR²⁰R²¹ and O,
wherein R¹⁹ is selected from H and 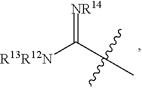
where each of R¹², R¹³ and R¹⁴ is as described previously,
each of R²⁰ and R²¹ is independently selected from H, NH₂ and 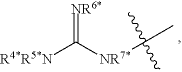
wherein each of R⁴*, R⁵*, R⁶* and R⁷* is as described previously, and
u is selected from 0, 1 and 2; and
g.

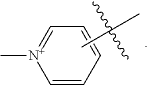

59. The pharmaceutical composition according to claim 58, wherein the compound of Formula A-I has the stereochemistry specified in Formula A-II 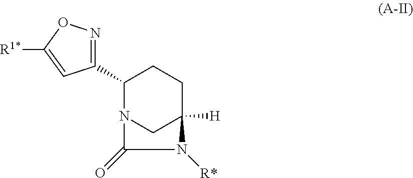
or a pharmaceutically acceptable salt thereof.

60. The pharmaceutical composition according to claim 59, wherein the compound, or a pharmaceutically acceptable salt thereof is selected from

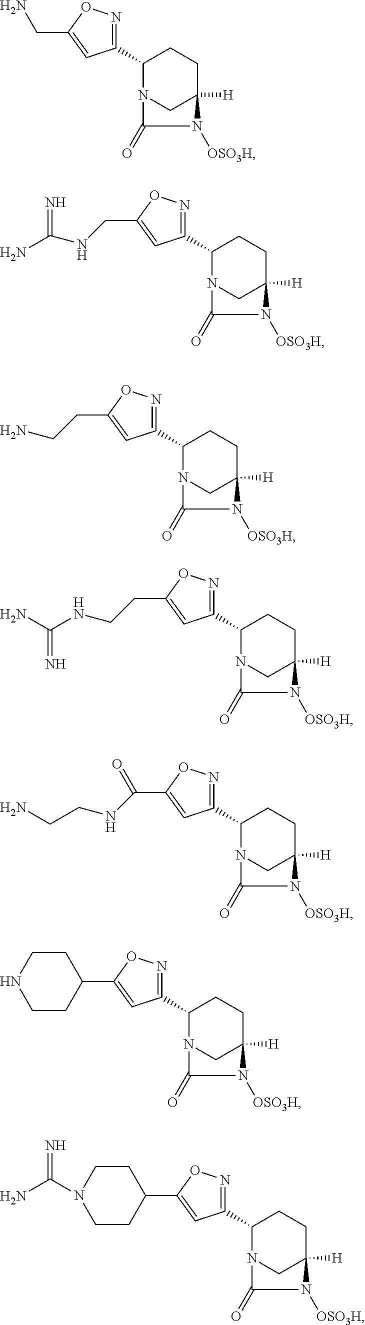

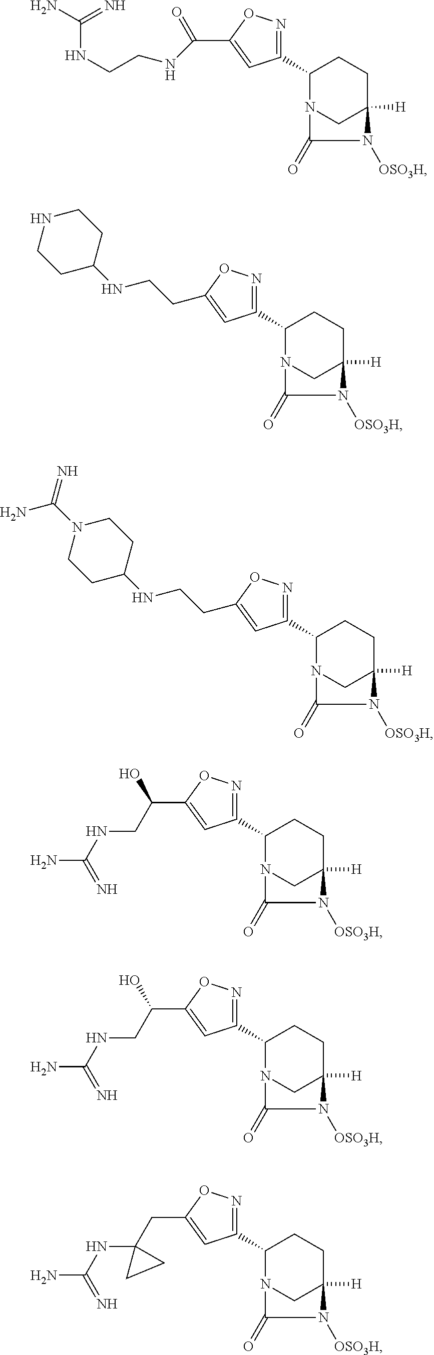

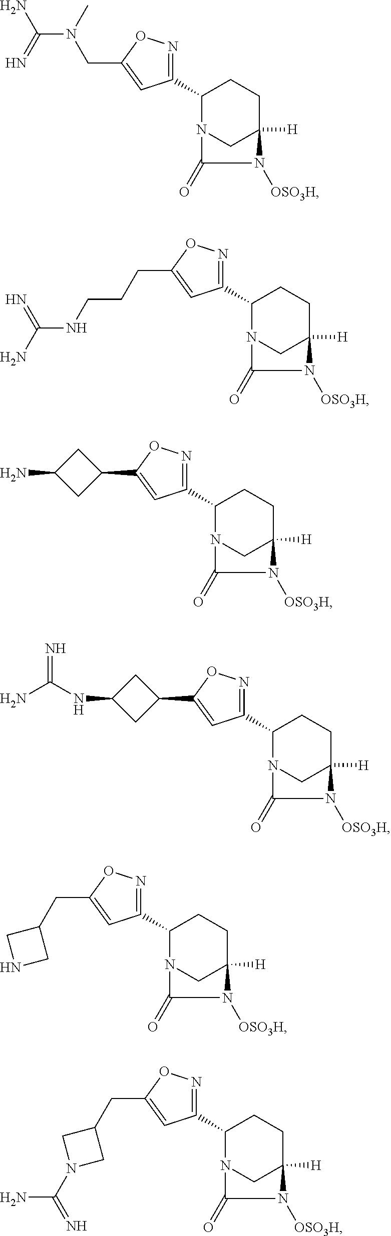

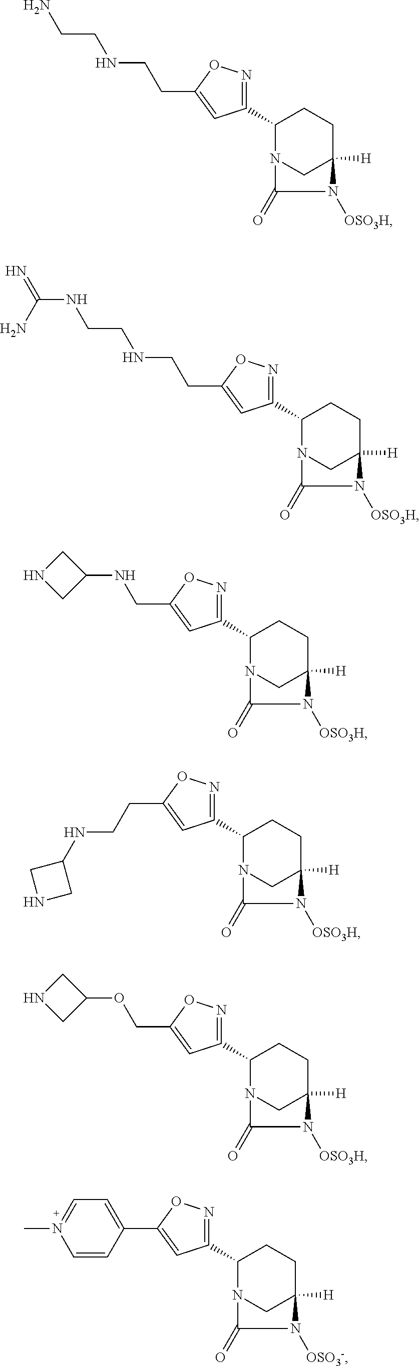

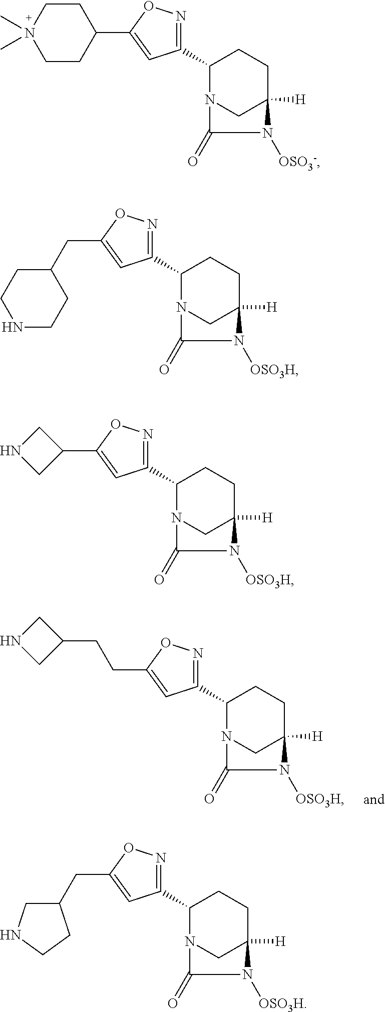

61. The pharmaceutical composition according to claim 58, wherein R¹* is selected from

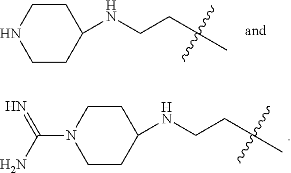

62. The pharmaceutical composition according to claim 60, wherein the compound is selected from

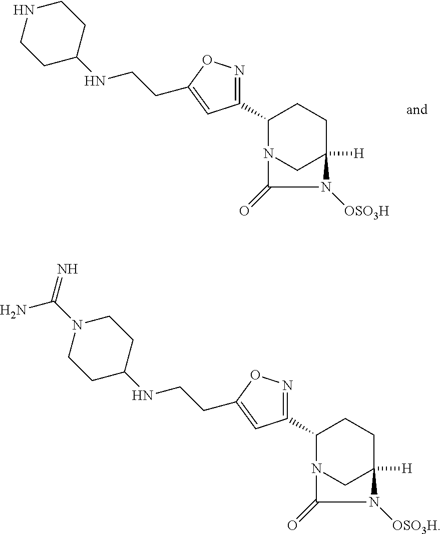

63. The pharmaceutical composition according to claim 62, wherein the compound or a pharmaceutically acceptable salt thereof has the Formula [chemical structure]


SEQUENCE LISTING (13 sequences)

SEQ ID NO: 1 - DNA, 32 bp, Artificial Sequence
  Sythetic primer pBR-Pbla
cgcatatgactcttcctttttcaatattattg

SEQ ID NO: 2 - DNA, 28 bp, Artificial Sequence
  Sythetic primer pBR-vec-1
gcggatccctgtcagaccaagtttactc

SEQ ID NO: 3 - DNA, 28 bp, Artificial Sequence
  Sythetic primer Pbla-cat
gccatatgatggagaaaaaaatcactgg

SEQ ID NO: 4 - DNA, 28 bp, Artificial Sequence
  Sythetic primer Vec-1-cat
cgggatccctagagaataggaacttcgg

SEQ ID NO: 5 - DNA, 4236 bp, Artificial Sequence
  Synthetic construct pBR-CBST-cat
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtcatatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatacggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaagtggcagggcggggcgtaaggcgcgccatttaaatgaagttcctattccgaagttcctattctctagggatccctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatttggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggattcacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 6 - DNA, 4391 bp, Artificial Sequence
  Synthetic construct pBR-CBST-KPC-2
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtcatatgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttccgtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgccatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagtgtatggcacggcaaatgactatgccgtcgtctggcccactgggcgcgcacctattgtgttggccgtctacacccgggcgcctaacaaggatgacaagcacagcgaggccgtcatcgccgctgcggctagactcgcgctcgagggattgggcgtcaacgggcagtaaggatccctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatttggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggattcacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 7 - DNA, 4116 bp, Artificial Sequence
  Synthetic construct pBR-CBST-CTX-M-15
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtcatatggaatctgttaaatcagcgagttgagatcaaaaaatctgaccttgttaactataatccgattgcggaaaagcacgtcaatgggacgatgtcactggctgagcttagcgcggccgcgctacagtacagcgataacgtggcgatgaataagctgattgctcacgttggcggcccggctagcgtcaccgcgttcgcccgacagctgggagacgaaacgttccgtctcgaccgtaccgagccgacgttaaacaccgccattccgggcgatccgcgtgataccacttcacctcgggcaatggcgcaaactctgcggaatctgacgctgggtaaagcattgggcgacagccaacgggcgcagctggtgacatggatgaaaggcaataccaccggtgcagcgagcattcaggctggactgcctgcttcctgggttgtgggggataaaaccggcagcggtggctatggcaccaccaacgatatcgcggtgatctggccaaaagatcgtgcgccgctgattctggtcacttacttcacccagcctcaacctaaggcagaaagccgtcgcgatgtattagcgtcggcggctaaaatcgtcaccgacggtttgtaaggatccctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatttggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggattcacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 8 - DNA, 4370 bp, Artificial Sequence
  Synthetic construct pBR-CBST-SHV-12
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtcatatgcgttatattcgcctgtgtattatctccctgttagccaccctgccgctggcggtacacgccagcccgcagccgcttgagcaaattaaacaaagcgaaagccagctgtcgggccgcgtaggcatgatagaaatggatctggccagcggccgcacgctgaccgcctggcgcgccgatgaacgctttcccatgatgagcacctttaaagtagtgctctgcggcgcagtgctggcgcgggtggatgccggtgacgaacagctggagcgaaagatccactatcgccagcaggatctggtggactactcgccggtcagcgaaaaacaccttgccgacggcatgacggtcggcgaactctgcgccgccgccattaccatgagcgataacagcgccgccaatctgctgctggccaccgtcggcggccccgcaggattgactgcctttttgcgccagatcggcgacaacgtcacccgccttgaccgctgggaaacggaactgaatgaggcgcttcccggcgacgcccgcgacaccactaccccggccagcatggccgcgaccctgcgcaagctgctgaccagccagcgtctgagcgcccgttcgcaacggcagctgctgcagtggatggtggacgatcgggtcgccggaccgttgatccgctccgtgctgccggcgggctggtttatcgccgataagaccggagctagcaagcggggtgcgcgcgggattgtcgccctgcttggcccgaataacaaagcagagcgcattgtggtgatttatctgcgggataccccggcgagcatggccgagcgaaatcagcaaatcgccgggatcggcgcggcgctgatcgagcactggcaacgctaaggatccctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatttggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggattcacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 9 - DNA, 4655 bp, Artificial Sequence
  Synthetic construct pBR-CBST-P99
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtcatatgatgagaaaatccctttgctgcgccctgctgctcggcatctcttgctctgctctcgccacgccagtgtcagaaaaacagctggcggaggtggtcgcgaatacgattaccccgctgatgaaagcccagtctgttccaggcatggcggtggccgttatttatcagggaaaaccgcactattacacatttggcaaggccgatatcgcggcgaataaacccgttacgcctcagaccctgttcgagctgggttctataagtaaaaccttcaccggcgttttaggtggggatgccattgctcgcggtgaaatttcgctggacgatgcggtgaccagatactggccacagctgacgggcaagcagtggcagggtattcgtatgctggatctcgccacctacaccgctggcggcctgccgctacaggtaccggatgaggtcacggataacgcctccctgctgcgcttttatcaaaactggcagccgcagtggaagcctggcacaacgcgtctttacgccaacgccagcatcggtctttttggtgcgctggcggtcaaaccttctggcatgccctatgagcaggccatgacgacgcgggtccttaagccgctcaagctggaccatacctggattaacgtgccgaaagcggaagaggcgcattacgcctggggctatcgtgacggtaaagcggtgcgcgtttcgccgggtatgctggatgcacaagcctatggcgtgaaaaccaacgtgcaggatatggcgaactgggtcatggcaaacatggcgccggagaacgttgctgatgcctcacttaagcagggcatcgcgctggcgcagtcgcgctactggcgtatcgggtcaatgtatcagggtctgggctgggagatgctcaactggcccgtggaggccaacacggtggtcgagggcagcgacagtaaggtagcactggcgccgttgcccgtggcagaagtgaatccaccggctcccccggtcaaagcgtcctgggtccataaaacgggctctactggcgggtttggcagctacgtggcctttattcctgaaaagcagatcggtattgtgatgctcgcgaatacaagctatccgaacccggcacgcgttgaggcggcataccatatcctcgaggcgctacagtaaggatccctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatttggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggattcacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 10 - DNA, 4337 bp, Artificial Sequence
  Synthetic construct pBR-CBST-OXA-15
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtcatatggcaatccgaatcttcgcgatacttttctccattttttctcttgccactttcgcgcatgcgcaagaaggcacgctagaacgttctgactggaggaagtttttcagcgaatttcaagccaaaggcacgatagttgtggcagacgaacgccaagcggatcgtgccatgttggtttttgatcctgtgcgatcgaagaaacgctactcgcctgcatcgacattcaagatacctcatacactttttgcacttgatgcaggcgctgttcgtgatgagttccagatttttcgatgggacggcgttaacaggggctttgcaggccacaatcaagaccaagatttgcgatcagcaatgcggaattctactgtttgggtgtatgagctatttgcaaaggaaattggtgatgacaaagctcggcgctatttgaagaaaatcgactatggcaacgccggtccttcgacaagtaatggcgattactggatagaaggcagccttgcaatctcggcgcaggagcaaattgcatttctcaggaagctctatcgtaacgagctgccctttcgggtagaacatcagcgcttggtcaaggatctcatgattgtggaagccggtcgcaactggatactgcgtgcaaagacgggctgggaaggccgtatgggttggtgggtaggatgggttgagtggccgactggctccgtattcttcgcactgaatattgatacgccaaacagaatggatgatcttttcaagagggaggcaatcgtgcgggcaatccttcgctctattgaagcgttaccgcccaacccggcagtcaactcggacgctgcgcgataaggatccctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatttggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggattcacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 11 - DNA, 4391 bp, Artificial Sequence
  Synthetic construct pBR-CBST-KPC-4
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtcatatgtcactgtatcgccgtctagttctgctgtcttgtctctcatggccgctggctggcttttctgccaccgcgctgaccaacctcgtcgcggaaccattcgctaaactcgaacaggactttggcggctccatcggtgtgtacgcgatggataccggctcaggcgcaactgtaagttaccgcgctgaggagcgcttcccactgtgcagctcattcaagggctttcttgctgccgctgtgctggctcgcagccagcagcaggccggcttgctggacacacccatccgttacggcaaaaatgcgctggttcggtggtcacccatctcggaaaaatatctgacaacaggcatgacggtggcggagctgtccgcggccgccgtgcaatacagtgataacgccgccgccaatttgttgctgaaggagttgggcggcccggccgggctgacggccttcatgcgctctatcggcgataccacgttccgtctggaccgctgggagctggagctgaactccgccatcccaggcgatgcgcgcgatacctcatcgccgcgcgccgtgacggaaagcttacaaaaactgacactgggctctgcactggctgcgccgcagcggcagcagtttgttgattggctaaagggaaacacgaccggcaaccaccgcatccgcgcggcggtgccggcagactgggcagtcggagacaaaaccggaacctgcggagggtatggcacggcaaatgactatgccgtcgtctggcccactgggcgcgcacctattgtgttggccgtctacacccgggcgcctaacaaggatgacaagcacagcgaggccgtcatcgccgctgcggctagactcgcgctcgagggattgggcgtcaacgggcagtaaggatccctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatttggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggattcacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 12 - DNA, 4649 bp, Artificial Sequence
  Synthetic construct pBR-CBST-DHA-1
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtcatatgaaaaaatcgttatctgcaacactgatttccgctctgctggcgttttccgccccggggttttctgccgctgataatgtcgcggcggtggtggacagcaccattaaaccgctgatggcacagcaggatattcccgggatggcggttgccgtctccgtaaagggtaagccctattatttcaattatggttttgccgatattcaggcaaaacagccggtcactgaaaatacactatttgagctcggatctgtaagtaaaactttcacaggtgtgctgggtgcggtttctgtggcgaaaaaagagatggcgctgaatgatccggcggcaaaataccagccggagctggctctgccgcagtggaaggggatcacattgctggatctggctacctataccgcaggcggactgccgttacaggtgccggatgcggtaaaaagccgtgcggatctgctgaatttctatcagcagtggcagccgtcccggaaaccgggcgatatgcgtctgtatgcaaacagcagtatcggcctgtttggtgctctgaccgcaaacgcggcggggatgccgtatgagcagttgctgactgcacgcatcctggcaccgctggggttatctcacacctttattactgtgccggaaagtgcgcaaagccagtatgcgtacggttataaaaacaaaaaaccggtccgcgtgtcgccgggacagcttgatgcggaatcttacggcgtgaaatccgcctcaaaagatatgctgcgctgggcggaaatgaatatggagccgtcacgggccggtaatgcggatctggaaatggcaatgtatctcgcccagacccgctactataaaaccgccgcgattaaccaggggctgggctgggaaatgtatgactggccgcagcagaaagatatgatcattaacggtgtgaccaacgaggtcgcattgcagccgcatccggtaacagacaaccaggttcagccgtataaccgtgcttcctgggtgcataaaacgggcgcaacaactggtttcggcgcctatgtcgcctttattccggaaaaacaggtggcgattgtgattctggcgaataaaaactacccgaataccgaaagagtcaaagctgcacaggctattttgagtgcactggaataaggatccctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatttggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggattcacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa

SEQ ID NO: 13 - DNA, 4664 bp, Artificial Sequence
  Synthetic construct pBR-CBST-ADC-33
ttcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtcatatgcgatttaaaaaaatttcttgtctacttttatccccgctttttatttttagtacctcaatttatgcgggcaatacaccaaaagaccaagaaattaaaaaactggtagatcaaaactttaaaccgttattagaaaaatatgatgtgccaggtatggctgtgggtgttattcaaaataataaaaagtatgaaatgtattatggtcttcaatctgttcaagataaaaaagccgtaaatagcagtaccatttttgagctaggttctgtcagtaaattatttaccgcgacagcaggtggatatgcaaaaaataaaggaaaaatctcttttgacgatacgcctggtaaatattggaaagaactaaaaaacacaccgattgaccaagttaacttacttcaactcgcgacgtatacaagtggtaaccttgccttgcagtttccagatgaagtaaaaacagaccaacaagttttaacttttttcaaagactggaaacctaaaaactcaatcggtgaatacagacaatattcaaatccaagtattggcctatttggaaaggttgtggctttgtctatgaataaacctttcgaccaagtcttagaaaaaacaatttttccggcccttggcttaaaacatagctatgtaaatgtacctaagacccagatgcaaaactatgcatttggttataaccaagaaaatcagccgattcgagttaaccgcggcccactcgatgccgcccctgcgtatggcgtcaaatcgacactacccgacatgttgagttttattcatgccaaccttaacccacagaaatatccggctgatattcaacgggcaattaatgaaacacatcaagggcgctatcaagtaaataccatgtatcaggcactcggttgggaagagttttcttatccggcaacgttacaaactttattagacagtaattcagaacagattgtgatgaaacctaataaagtgactgctatttcaaaggaaccttcagttaagatgtaccataaaactggctcaaccaacggtttcggaacgtatgtagtgtttattcctaaagaaaatattggcttagtcatgttaaccaataaacgtattccaaatgaagagcgcattaaggcagcttatgctgtgctgaatgcaataaagaaataaggatccctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatttggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggattcacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaa